(19) United States
(12) Patent Application Publication
(10) Pub. No.: US 2025/0368609 A1
(43) Pub. Date: Dec. 4, 2025

(54) TRIAZINANE COMPOUND AND COMPOSITION CONTAINING SAME

(71) Applicant: SUMITOMO CHEMICAL COMPANY, LIMITED, Tokyo (JP)

(72) Inventors: Motohiro NAKAJIMA, Takarazuka-shi, Hyogo (JP); Yoshinobu JIN, Kasai-shi, Hyogo (JP)

(73) Assignee: SUMITOMO CHEMICAL COMPANY, LIMITED, Tokyo (JP)

(21) Appl. No.: 18/875,513

(22) PCT Filed: Jun. 21, 2023
(86) PCT No.: PCT/JP2023/022878

§ 371 Date: Dec. 16, 2024

(30) Foreign Application Priority Data
Jun. 24, 2022 (JP) 2022-101773

(51) Int. Cl. C07D 251/26; A01N 53/00

(52) U.S. Cl. CPC C07D 251/26; A01N 53/00

(57) ABSTRACT

The present invention provides a compound having an excellent control effect on weeds. A compound represented by a formula (I), or an N-oxide of the compound, or a salt of the compound or the N-oxide have an excellent control effect on weeds.

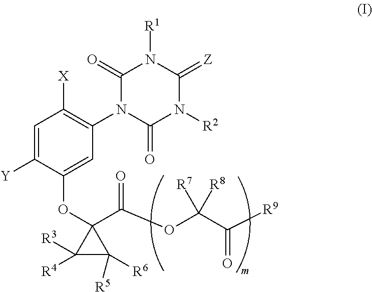

### TECHNICAL FIELD

The present invention relates to a triazinane compound and an herbicidal composition containing the same.

### BACKGROUND ART

Various compounds have been studied and put to practical use for the purpose of controlling weeds.

It is also known that certain compounds have a weed control effect (refer to, for example, Patent Literature 1 and Patent Literature 2).

### CITATION LIST

### Patent Literature

Patent Literature 1: WO 00/50409 A
Patent Literature 2: U.S. Pat. No. 6,403,534

### SUMMARY OF INVENTION

### Technical Problem

An object of the present invention is to provide a compound having an excellent control effect on weeds.

### Solution to Problem

The present invention will be described as follows.

[1] A compound represented by a formula (I)

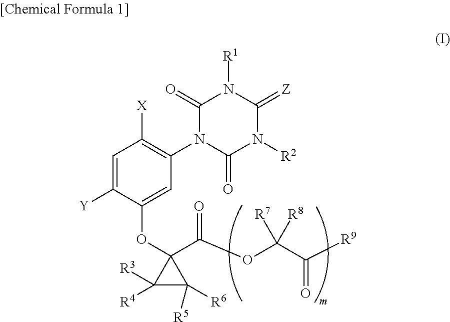

[wherein
X represents a hydrogen atom or a halogen atom,
Y represents a halogen atom,
Z represents an oxygen atom or a sulfur atom,
R¹ and R² are the same as or different from each other and represent a hydrogen atom or a C1-C3 chain hydrocarbon group,
R³, R⁴, R⁵, and R⁶ are the same as or different from each other and represent a hydrogen atom or a C1-C3 alkyl group,
R⁷ and R⁸ are the same as or different from each other and represent a hydrogen atom or a C1-C3 alkyl group, or
R⁷ and R⁸ are taken together with a carbon atom to which they are attached to form a C3-C6 cycloalkyl group,
R⁹ represents —OR¹⁰, —SR¹⁰, or —NR¹¹R¹²,
R¹⁰ represents a C1-C8 chain hydrocarbon group optionally substituted with one or more substituents selected from a group A, a C3-C8 alicyclic hydrocarbon group optionally substituted with one or more substituents selected from a group B, or a hydrogen atom,
R¹¹ represents a hydrogen atom, a C1-C8 chain hydrocarbon group, or a C3-C8 alicyclic hydrocarbon group,
R¹² represents a C1-C8 chain hydrocarbon group optionally substituted with one or more substituents selected from the group A, a C3-C8 alicyclic hydrocarbon group optionally substituted with one or more substituents selected from the group B, a phenyl group optionally substituted with one or more substituents selected from a group C, a 5-6 membered aromatic heterocyclic group optionally substituted with one or more substituents selected from the group C, or a hydrogen atom, and
m represents 0 or 1;

The group A: a group consisting of a phenyl group optionally substituted with one or more substituents selected from the group C, a 5-6 membered aromatic heterocyclic group optionally substituted with one or more substituents selected from the group C, a halogen atom, a cyano group, a C1-C3 alkoxy group, and a C1-C3 alkylthio group;

The group B: a group consisting of an oxo group, a thioxo group, a halogen atom, a cyano group, a C1-C3 alkoxy group, and a C1-C3 alkylthio group;

The group C: a group consisting of a C1-C3 chain hydrocarbon group optionally substituted with one or more halogen atoms, a C1-C3 alkoxy group optionally substituted with one or more halogen atoms, a C1-C3 alkylthio group optionally substituted with one or more halogen atoms, a halogen atom, a cyano group, and a nitro group],
or an N-oxide of the compound, or a salt of the compound or the N-oxide.
(hereinafter, the compound represented by the formula (I), or the N-oxide of the compound, or the salt of the compound or the N-oxide is referred to as a compound of the present invention).

[2] The compound, or the N-oxide of the compound, or the salt of the compound or the N-oxide according to [1], wherein Z is a sulfur atom.

[3] The compound, or the N-oxide of the compound, or the salt of the compound or the N-oxide according to [1] or [2], wherein R³, R⁴, R⁵, and R⁶ are each a hydrogen atom.

[4] The compound, or the N-oxide of the compound, or the salt of the compound or the N-oxide according to any one of [1] to [3], wherein X is a fluorine atom, and Y is a chlorine atom.

[5] The compound, or the N-oxide of the compound, or the salt of the compound or the N-oxide according to any one of [1] to [4], wherein R¹ and R² are each a methyl group.

[6] The compound, or the N-oxide of the compound, or the salt of the compound or the N-oxide according to any one of [1] to [5],
wherein
   R⁷ and R⁸ are the same as or different from each other and represent a hydrogen atom, a methyl group or an ethyl group, or
   R⁷ and R⁸ are taken together with a carbon atom to which they are attached to form a C3-C4 cycloalkyl group,
   R¹⁰ is a C1-C3 chain hydrocarbon group optionally substituted with one or more substituents selected from the group A or a hydrogen atom,
   R¹¹ is a hydrogen atom or a C1-C3 chain hydrocarbon group, and
   R¹² is a C1-C3 chain hydrocarbon group optionally substituted with one or more substituents selected from the group A or a hydrogen atom.

[7] The compound, or the N-oxide of the compound, or the salt of the compound or the N-oxide according to any one of [1] to [6], wherein R⁹ is —OR¹⁰ or —NR¹¹R¹².

[8] The compound, or the N-oxide of the compound, or the salt of the compound or the N-oxide according to any one of [1] to [6], wherein R⁹ is —OR¹⁰.

[9] The compound, or the N-oxide of the compound, or the salt of the compound or the N-oxide according to any one of [1] to [8], wherein m is 0.

[10] The compound, or the N-oxide of the compound, or the salt of the compound or the N-oxide according to any one of [1] to [8], wherein m is 1.

[11] An herbicidal composition comprising: the compound, or the N-oxide of the compound, or the salt of the compound or the N-oxide according to any one of [1] to [10]; and an inert carrier.

[12] A method of controlling a weed, comprising a step of applying, to the weed or soil in which the weed grows, the compound, or the N-oxide of the compound, or the salt of the compound or the N-oxide according to any one of [1] to [10].

[13] Use of the compound, or the N-oxide of the compound, or the salt of the compound or the N-oxide according to any one of [1] to [10], for controlling a weed.

[14] A composition comprising: one or more components selected from a group consisting of a group (d) and a group (f); and the compound, or the N-oxide of the compound, or the salt of the compound or the N-oxide according to any one of [1] to [10];

The group (d): phytotoxicity alleviating ingredients

The group (f): herbicidally active ingredients
(hereinafter, also referred to as the composition of the present invention).

### Advantageous Effects of Invention

According to the present invention, weeds can be controlled.

### DESCRIPTION OF EMBODIMENTS

A halogen atom in the present invention means a fluorine atom, a chlorine atom, a bromine atom, or an iodine atom.

The notation of “CU-CW” in the present specification means that the number of carbon atoms is U to W. For example, the notation “C1-C8” means that the number of carbon atoms is 1 to 8.

A chain hydrocarbon group in the present invention represents, for example, an alkyl group, an alkenyl group, or an alkynyl group.

Examples of the alkyl group include a methyl group, an ethyl group, a propyl group, an isopropyl group, a 1,1-dimethylpropyl group, a 1,2-dimethylpropyl group, a 1-ethylpropyl group, a butyl group, a sec-butyl group, a tert-butyl group, a pentyl group, a hexyl group, and an octyl group.

Examples of the alkenyl group include a vinyl group, a 1-propenyl group, a 2-propenyl group, a 1-methyl-1-propenyl group, a 1-methyl-2-propenyl group, a 1,2-dimethyl-1-propenyl group, a 1-ethyl-2 propenyl group, a 3-butenyl group, a 4-pentenyl group, a 5-hexenyl group, and a 7-octenyl group.

Examples of the alkynyl group include an ethynyl group, a 1-propynyl group, a 2-propynyl group, a 1-methyl-2-propynyl group, a 1,1-dimethyl-2-propynyl group, a 1-ethyl-2-propynyl group, a 2-butynyl group, a 4-pentynyl group, a 5-hexynyl group, and a 7-octynyl group.

Examples of the alicyclic hydrocarbon group in the present invention include a cycloalkyl group and a cycloalkenyl group.

Examples of the cycloalkyl group include a cyclopropyl group, a cyclobutyl group, a cyclopentyl group, a cyclohexyl group, and a cyclooctyl group.

Examples of the cycloalkenyl group include a cyclopentenyl group, a cyclohexenyl group, and a cyclooctenyl group.

Examples of the alkoxy group include a methoxy group, an ethoxy group, a propoxy group, and an isopropoxy group.

Examples of the alkylthio group include a methylthio group, an ethylthio group, a propylthio group, and an isopropylthio group.

Examples of a 5-6 membered aromatic heterocyclic group in the present invention include 5-membered aromatic heterocyclic groups such as a pyrrolyl group, a furanyl group, a thienyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, and a thiadiazolyl group; and 6-membered aromatic heterocyclic groups such as a pyridyl group, a pyridazinyl group, a pyrimidinyl group, a pyrazinyl group, a triazinyl group, and a tetrazinyl group.

When a substituent is substituted with two or more halogen atoms, the halogen atoms may be the same as each other or different from each other.

When the chain hydrocarbon group, the alicyclic hydrocarbon group, the phenyl group, or the 5-6 membered aromatic heterocyclic group is substituted with two or more groups or atoms selected from a specific group (for example, a group consisting of a C1-C8 alkyl group and a halogen atom), these groups or atoms may be the same as each other or different from each other.

In the notation of “optionally substituted with one or more substituents selected from a group T” (T means any one of A, B, and C) in the present specification, when there are two or more substituents selected from the group T, the substituents may be the same as each other or different from each other.

The compound of the present invention may have one or more stereoisomers. Examples of the stereoisomers include enantiomers, diastereomers, atropisomers, and geometric isomers. The present invention includes each stereoisomer and any ratio of stereoisomer mixtures.

The compound represented by the formula (I) or an N oxide thereof may be mixed with an acid such as hydrochloric acid, sulfuric acid, nitric acid, phosphoric acid, acetic acid, or benzoic acid to form an acid addition salt such as a hydrochloride, a sulfate, a nitrate, a phosphate, an acetate or a benzoate.

Examples of an embodiment of a compound of the present invention include the following compounds.

[Embodiment 1] In the compounds of the present invention, a compound wherein Z is a sulfur atom.

[Embodiment 2] In the compounds of the present invention, a compound wherein R³, R⁴, R⁵, and R⁶ are each independently a hydrogen atom or a methyl group.

[Embodiment 3] In the compounds of the present invention, a compound wherein R³, R⁴, R⁵, and R⁶ are each a hydrogen atom.

[Embodiment 4] In the compounds of the present invention, a compound wherein X and Y are the same as or different from each other and are each a halogen atom.

[Embodiment 5] In the compounds of the present invention, a compound wherein X is a fluorine atom and Y is a chlorine atom.

[Embodiment 6] In the compounds of the present invention, a compound wherein R¹ and R² are each a methyl group.

[Embodiment 7] In the compounds of the present invention, a compound wherein Z is a sulfur atom, R³, R⁴, R⁵, and R⁶ are the same as or different from each other and are each a hydrogen atom or each a methyl group, and X and Y are the same as or different from each other and are each a halogen atom.

[Embodiment 8] In the compounds of the present invention, a compound wherein Z is a sulfur atom, R³, R⁴, R⁵, and R^{e} are each a hydrogen atom, X is a fluorine atom, and Y is a chlorine atom.

[Embodiment 9] In the compounds of the present invention, a compound wherein Z is a sulfur atom, R³, R⁴, R⁵, and R⁶ are each a hydrogen atom, X is a fluorine atom, Y is a chlorine atom, and R¹ and R² are each a methyl group.

[Embodiment 10] In the compounds of the present invention, a compound wherein m is 1, and R⁷ and R⁸ are the same as or different from each other and are each a hydrogen atom or a C1-C3 alkyl group, or are taken together with the carbon atom to which they are attached to form a C3-C6 cycloalkyl group.

[Embodiment 11] In the compounds of the present invention, a compound wherein m is 1, and R⁷ and R⁸ are the same as or different from each other and are each a hydrogen atom, a methyl group or an ethyl group, or a cyclopropyl group is formed by being taken together with the carbon atom to which they are attached.

[Embodiment 12] In the compounds of the present invention, a compound wherein m is 1, and R⁷ and R⁸ are the same as or different from each other and are each a hydrogen atom or a methyl group.

[Embodiment 13] In the compounds of the present invention, a compound wherein R¹⁰ is a C1-C3 chain hydrocarbon group optionally substituted with one or more substituents selected from a group A, a C3-C6 cycloalkyl group optionally substituted with one or more substituents selected from a group B, or a hydrogen atom.

[Embodiment 14] In the compounds of the present invention, a compound wherein R¹⁰ is a C1-C3 chain hydrocarbon group optionally substituted with one or more substituents selected from the group A or a hydrogen atom.

[Embodiment 15] In the compounds of the present invention, a compound wherein R¹⁰ is a C1-C3 chain hydrocarbon group, a C1-C3 alkoxy C1-C3 alkyl group, a C1-C3 alkylthio C1-C3 alkyl group, or a benzyl group.

[Embodiment 16] In the compounds of the present invention, a compound wherein R¹⁰ is a hydrogen atom, a methyl group, an ethyl group, a propyl group, an isopropyl group, a 2-propenyl group, a 2-propynyl group, or a benzyl group.

[Embodiment 17] In the compounds of the present invention, a compound wherein R¹⁰ is a hydrogen atom, a methyl group, or an ethyl group.

[Embodiment 18] In the compounds of the present invention, a compound wherein R¹¹ is a hydrogen atom or a C1-C3 chain hydrocarbon group.

[Embodiment 19] In the compounds of the present invention, a compound wherein R¹¹ is a hydrogen atom, a methyl group, or an ethyl group.

[Embodiment 20] In the compounds of the present invention, a compound wherein R¹² is a C1-C3 chain hydrocarbon group optionally substituted with one or more substituents selected from the group A, a C3-C6 cycloalkyl group optionally substituted with one or more substituents selected from the group B, a phenyl group optionally substituted with one or more substituents selected from a group C, or a hydrogen atom.

[Embodiment 21] In the compounds of the present invention, a compound wherein R¹² is a C1-C3 chain hydrocarbon group optionally substituted with one or more substituents selected from the group A or a hydrogen atom.

[Embodiment 22] In the compounds of the present invention, a compound wherein R¹² is a C1-C3 chain hydrocarbon group, a C1-C3 alkoxy C1-C3 alkyl group, a C1-C3 alkylthio C1-C3 alkyl group, or a benzyl group.

[Embodiment 23] In the compounds of the present invention, a compound wherein R¹² is a hydrogen atom, a methyl group, an ethyl group, a propyl group, an isopropyl group, a 2-propenyl group, a 2-propynyl group, or a benzyl group.

[Embodiment 24] In the compounds of the present invention, a compound wherein R¹² is a hydrogen atom, a methyl group, or an ethyl group.

[Embodiment 25] In the embodiments 1 to 9, a compound wherein R⁹ is —OR¹⁰, and m is 1.

[Embodiment 26] In the embodiments 1 to 9, a compound wherein R⁹ is —NR¹¹R¹², and m is 1.

[Embodiment 27] In the embodiments 1 to 9, a compound wherein R⁹ is —OR¹⁰, and m is 0.

[Embodiment 28] In the embodiments 1 to 9, a compound wherein R⁹ is —NR¹¹R¹², and m is 0.

[Embodiment 29] In the embodiment 25, a compound wherein R⁷ and R⁸ are the same as or different from each other and are each a hydrogen atom, a methyl group, or an ethyl group, or R⁷ and R⁸ are taken together with the carbon atom to which they are attached to form a cyclopropyl group, and R¹⁰ is a hydrogen atom, a methyl group, an ethyl group, a propyl group, an isopropyl group, a 2-propenyl group, a 2-propynyl group, or a benzyl group.

[Embodiment 30] In the embodiment 25, a compound wherein R⁷ and R⁸ are the same as or different from each other and are each a hydrogen atom or a methyl group, and R¹⁰ is a hydrogen atom, a methyl group, or an ethyl group.

[Embodiment 31] In the embodiment 26, a compound wherein R⁷ and R⁸ are the same as or different from each other and are each a hydrogen atom, a methyl group, or an ethyl group, or R⁷ and R⁸ are taken together with the carbon atom to which they are attached to form a cyclopropyl group,
R¹¹ is a hydrogen atom, a methyl group, or an ethyl group, and
R¹² is a hydrogen atom, a methyl group, an ethyl group, a propyl group, an isopropyl group, a 2-propenyl group, a 2-propynyl group, or a benzyl group.

[Embodiment 32] In the embodiment 26, a compound wherein R⁷ and R⁸ are the same as or different from each other and are each a hydrogen atom or a methyl group, R¹¹ is a hydrogen atom, a methyl group, or an ethyl group, and R¹² is a hydrogen atom, a methyl group, or an ethyl group.

[Embodiment 33] In the embodiment 27, a compound wherein R¹⁰ is a hydrogen atom, a methyl group, an ethyl group, a propyl group, an isopropyl group, a 2-propenyl group, a 2-propynyl group, or a benzyl group.

[Embodiment 34] In the embodiment 27, a compound wherein R¹⁰ is a hydrogen atom, a methyl group, or an ethyl group.

[Embodiment 35] In the embodiment 28, a compound wherein R¹¹ is a hydrogen atom, a methyl group, or an ethyl group, and R¹² is a hydrogen atom, a methyl group, an ethyl group, a propyl group, an isopropyl group, a 2-propenyl group, a 2-propynyl group, or a benzyl group.

[Embodiment 36] In the embodiment 28, a compound wherein R¹¹ is a hydrogen atom, a methyl group, or an ethyl group, and R¹² is a hydrogen atom, a methyl group, or an ethyl group.

[Embodiment 37] In the compounds of the present invention, a compound wherein Z is a sulfur atom, R³, R⁴, R⁵, and R⁶ are each a hydrogen atom, X is a fluorine atom, Y is a chlorine atom, R¹ and R² are each a methyl group, R⁷ and R⁸ are the same as or different from each other and are each a hydrogen atom, a methyl group, or an ethyl group, or are taken together with the carbon atom to which they are attached to form a cyclopropyl group, R⁹ is —OR¹⁰ or —NR¹¹R¹², R¹⁰ is a hydrogen atom, a C1-C4 alkyl group, a C1-C4 alkenyl group, a C1-C4 alkynyl group or a benzyl group, R¹¹ is a hydrogen atom or a C1-C3 alkyl group, and R¹² is a C1-C3 alkyl group.

[Embodiment 38] In the compounds of the present invention, a compound wherein Z is a sulfur atom, R³, R⁴, R⁵, and R⁶ are each a hydrogen atom, X is a fluorine atom, Y is a chlorine atom, R¹ and R² are each a methyl group, R⁷ and R⁸ are the same as or different from each other and are each a hydrogen atom, a methyl group, or an ethyl group, R⁹ is —OR¹⁰ or —NR¹¹R¹², R¹⁰ is a hydrogen atom, a C1-C4 alkyl group, a C1-C4 alkenyl group, a C1-C4 alkynyl group, or a benzyl group, and R¹¹ and R¹² are the same as or different from each other and are each a C1-C3 alkyl group.

Next, a description will be given as to a production method of the compound of the present invention.

### Production Method A

A compound represented by a formula (I-1) (hereinafter, referred to as a compound of the present invention (I-1)), a compound represented by a formula (I-2) (hereinafter, referred to as a compound of the present invention (1-2)), a compound represented by a formula (I-3) (hereinafter, referred to as a compound of the present invention (1-3)) and a compound represented by a formula (I) (hereinafter, referred to as a compound of the present invention (1)) can be produced according to the following scheme.

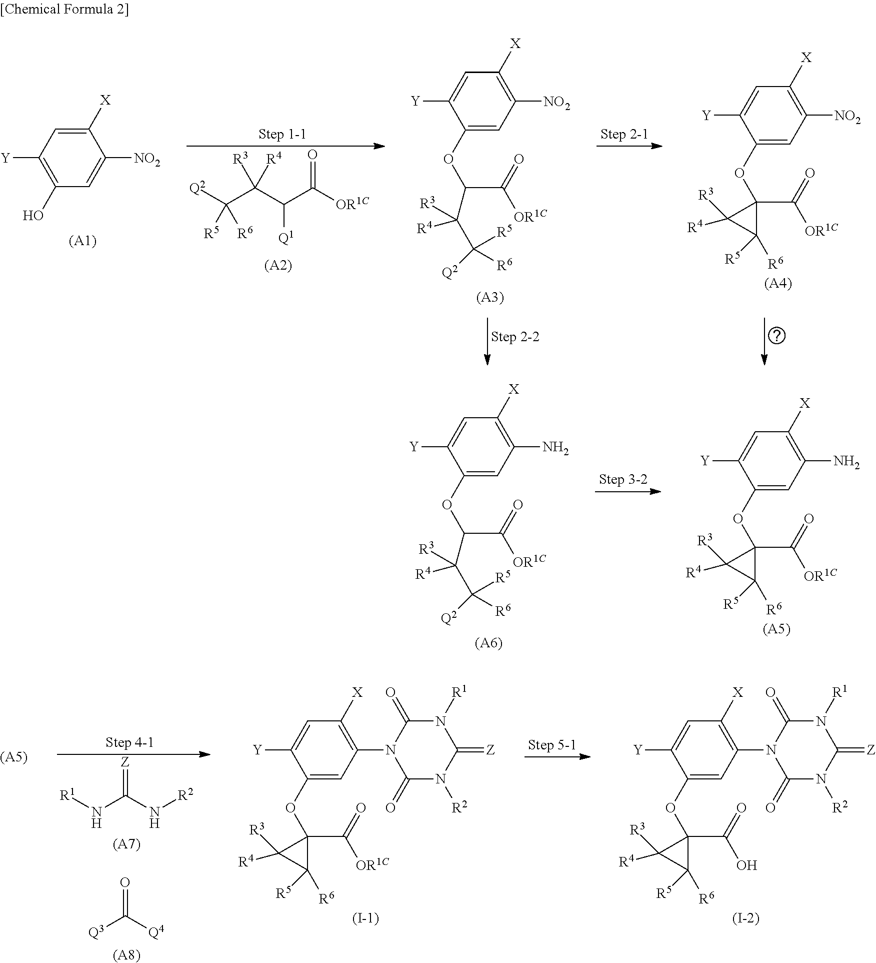

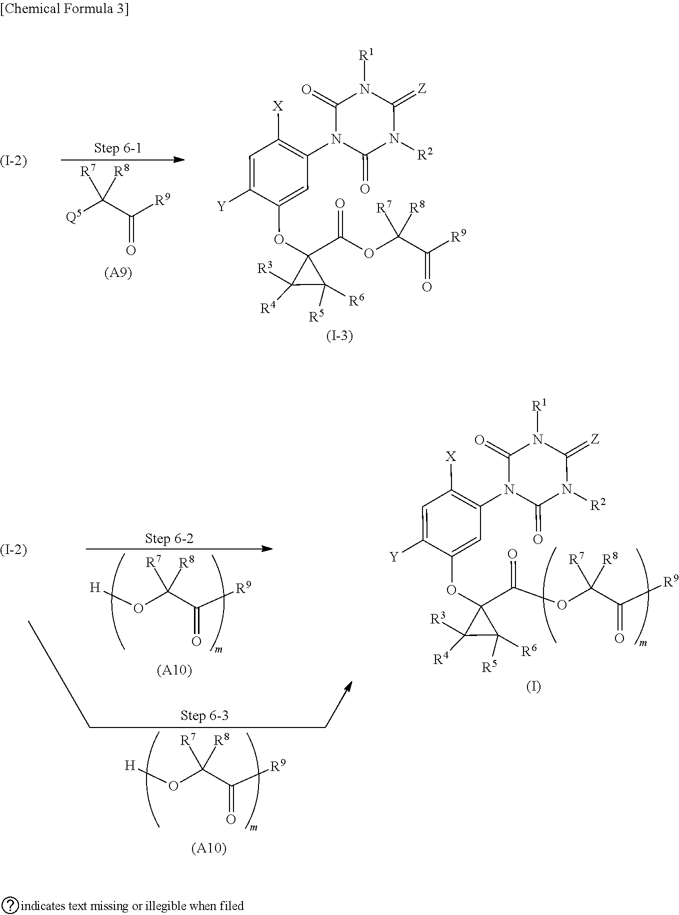

[In the formula, Q¹ represents a leaving group such as a chlorine atom, a bromine atom, an iodine atom, a methanesulfonyloxy group, or a 4-methylphenylsulfonyloxy group, Q² represents a leaving group such as a chlorine atom, a bromine atom, an iodine atom, a methanesulfonyloxy group, or a 4-methylphenylsulfonyloxy group, Q³ and Q⁴ are the same as or different from each other and represent a leaving group such as a chlorine atom, a bromine atom, an iodine atom, an imidazolyl group, or a triazolyl group, Q⁵ represents a leaving group such as a chlorine atom, a bromine atom, an iodine atom, a methanesulfonyloxy group or a 4-methylphenylsulfonyloxy group, and other symbols represent the same meaning as described above.]

Hereinafter, each step will be described.

### Production Method A-1

A compound represented by a formula (A3) (hereinafter, referred to as a compound (A3)) can be produced by a step of reacting a compound represented by a formula (A1) (hereinafter, referred to as a compound (A1)) with a compound represented by a formula (A2) (hereinafter, referred to as a compound (A2)) in the presence of a base (hereinafter, referred to as a step 1-1).

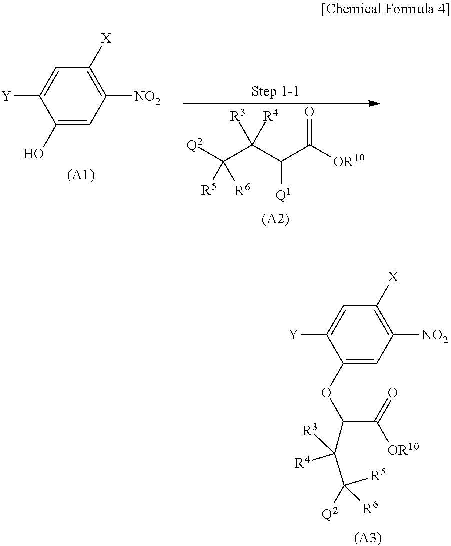

[In the formula, symbols represent the same meaning as described above.]

The reaction is usually performed in a solvent. Examples of the solvent used in the reaction include hydrocarbons such as toluene, xylene, and hexane (hereinafter, referred to as hydrocarbons); ethers such as methyl tert-butyl ether (hereinafter, referred to as MTBE), tetrahydrofuran (hereinafter, referred to as THF), and 1,4-dioxane (hereinafter, referred to as ethers); halogenated hydrocarbons such as dichloromethane, chloroform, and chlorobenzene (hereinafter, referred to as halogenated hydrocarbons); amides such as N, N-dimethylformamide (hereinafter, referred to as DMF) (hereinafter, referred to as amides); imidazolinones such as 1,3-dimethyl-2-imidazolinone (hereinafter, referred to as imidazolinones); sulfoxide such as dimethyl sulfoxide (hereinafter, referred to as DMSO) (hereinafter, referred to as sulfoxides); nitrile such as acetonitrile (hereinafter, referred to as nitriles); water; and mixtures of two or more kinds thereof.

Examples of the base used in the reaction include organic bases such as triethylamine, diisopropylethylamine, 1,8-diazabicyclo[5.4.0]undec-7-ene (DBU), pyridine, and 4-(dimethylamino)pyridine (hereinafter, referred to as organic bases); alkali metal carbonates such as sodium carbonate and potassium carbonate (hereinafter, referred to as alkali metal carbonates); alkaline earth metal carbonates such as magnesium carbonate and calcium carbonate (hereinafter, referred to as an alkaline earth metal carbonates); alkali metal hydrogen carbonates such as sodium hydrogen carbonate and potassium hydrogen carbonate (hereinafter, referred to as alkali metal hydrogen carbonates); metal carboxylates such as metal acetates (here, the metal acetates refer to metal acetates such as sodium acetate, potassium acetate, calcium acetate, and magnesium acetate) (hereinafter, referred to as metal carboxylates); metal alkoxides such as sodium methoxide, sodium ethoxide, sodium t-butoxide, potassium methoxide, and potassium t-butoxide (hereinafter, referred to as metal alkoxides); metal hydroxides such as sodium hydroxide, potassium hydroxide, lithium hydroxide, calcium hydroxide, and magnesium hydroxide (hereinafter, referred to as metal hydroxides); and metal hydrides such as lithium hydride, sodium hydride, potassium hydride, and calcium hydride (hereinafter, referred to as metal hydrides).

In the reaction, the compound (A2) is usually used in a ratio of 1 to 10 mol, and the base is usually used in a ratio of 1 to 10 mol, with respect to 1 mol of the compound (A1).

The reaction temperature is usually within the range of −80 to 150° C. The reaction time is usually within the range of 0.1 to 120 hours.

After completion of the reaction, the reaction mixture is mixed with water, and then the mixture is extracted with an organic solvent. Thereafter, an organic layer is subjected to post-treatment operations such as washing, drying, and concentration, thereby making it possible to obtain the compound (A3).

The compound (A1) is a publicly known compound, or can be produced according to a method described in the literature such as EP 0061741 A and a method similar thereto.

The compound (A2) is a publicly known compound, or can be produced according to a method described in the literature such as Angewandte Chemie vol. 97, No. 7, pages 599 and 600 (1985) and a method similar thereto.

### Production Method A-2

A compound represented by the formula (A4) (hereinafter, referred to as a compound (A4)) can be produced by a step of cyclizing the compound (A3) in the presence of a base (hereinafter, referred to as a step 2-1).

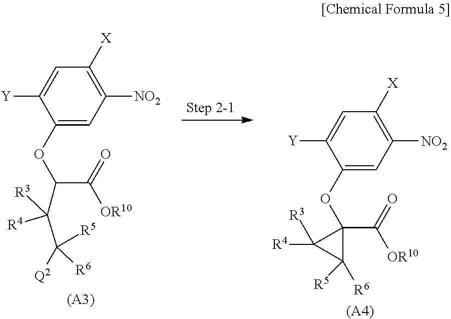

[In the formula, symbols represent the same meaning as described above.]

The reaction of the step 2-1 is usually performed in a solvent. Examples of the solvent used in the reaction of the step 2-1 include hydrocarbons; ethers; amides; imidazolinones; sulfoxides; secondary alcohols such as i-propanol (hereinafter, referred to as secondary alcohols); tertiary alcohol such as t-butanol (hereinafter, referred to as tertiary alcohols); and mixtures of two or more kinds thereof.

Examples of the base used in the reaction of the step 2-1 include metal alkoxides and metal hydrides.

In the reaction of the step 2-1, the base is usually used in a ratio of 1 to 10 mol with respect to 1 mol of the compound (A3).

The reaction temperature in the step 2-1 is usually in the range of −80 to 150° C. The reaction time in the step 2-1 is usually within the range of 0.1 to 120 hours.

After completion of the reaction in the step 2-1, the reaction mixture is mixed with water, and then the mixture is extracted with an organic solvent. Thereafter, an organic layer is subjected to post-treatment operations such as washing, drying, and concentration, thereby making it possible to obtain the compound (A4).

### Production Method A-3

A compound represented by a formula (A5) (hereinafter, referred to as a compound (A5)) can be produced by a step of reducing the compound (A4) using a metal such as iron (for example, iron powder), zinc or tin and an acid (hereinafter, referred to as a step 3-1).

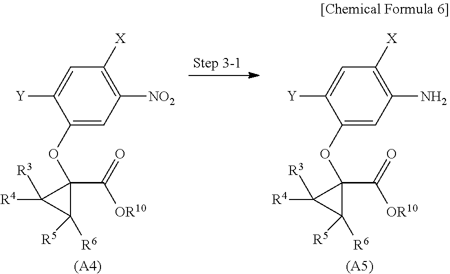

[In the formula, symbols represent the same meaning as described above.]

The reaction of the step 3-1 is usually performed in a solvent. Examples of the solvent used in the reaction of the step 3-1 include an ester such as ethyl acetate (hereinafter, referred to as esters); hydrocarbons; water; and mixtures of two or more kinds thereof.

Examples of the acid used in the reaction of the step 3-1 include inorganic acids (hereinafter, referred to as inorganic acids) such as sulfuric acid and hydrochloric acid; organic acids such as acetic acid (hereinafter, referred to as organic acids); acidic aqueous solution such as iron (III) chloride aqueous solution, iron (III) sulfate aqueous solution, and iron (III) acetylacetonate aqueous solution; and mixtures of two or more kinds thereof.

In the reaction of the step 3-1, the metal is usually used in a ratio of 3 to 100 mol, and the acid is usually used in an excess amount of 1 mol, with respect to 1 mol of the compound (A4).

The reaction temperature in the step 3-1 is usually in the range of −20 to 150° C. The reaction time in the step 3-1 is usually within the range of 0.1 to 120 hours.

After completion of the reaction in the step 3-1, the reaction mixture is mixed with water, and then the mixture is extracted with an organic solvent. Thereafter, an organic layer is subjected to post-treatment operations such as washing, drying, and concentration, thereby making it possible to obtain the compound (A5).

### Production Method A-4

The compound (A5) can also be produced by a step of reducing the compound (A3) using a metal such as iron (for example, iron powder), zinc, or tin and an acid to obtain a compound (A6) (hereinafter, referred to as a step 2-2), and a step of cyclizing the compound (A6) in the presence of a base to obtain the compound (A5) (hereinafter, referred to as a step 3-2).

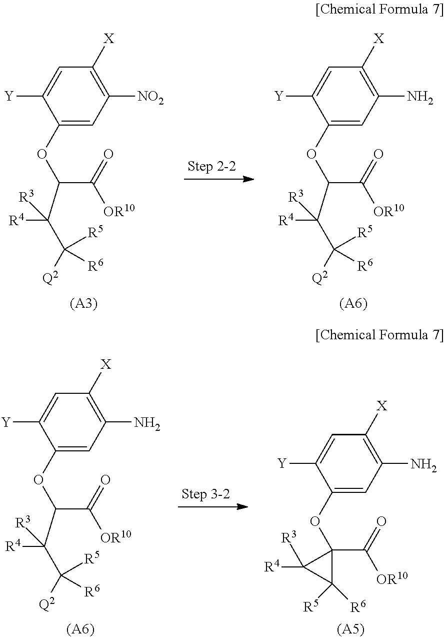

[In the formula, symbols represent the same meaning as described above.]

The reaction of the step 2-2 is usually performed in a solvent. Examples of the solvent used in the reaction of the step 2-2 include esters; hydrocarbons; water; and mixtures of two or more kinds thereof.

Examples of the acid used in the reaction of the step 2-2 include inorganic acids; organic acids; acidic aqueous solution such as iron (III) chloride aqueous solution, iron (III) sulfate aqueous solution, and iron (III) acetylacetonate aqueous solution; and mixtures of two or more kinds thereof.

In the reaction of the step 2-2, the metal is usually used in a ratio of 3 to 100 mol, and the acid is usually used in an excess amount of 1 mol, with respect to 1 mol of the compound (A3).

The reaction temperature in the step 2-2 is usually in the range of −20 to 150° C. The reaction time in the step 2-2 is usually within the range of 0.1 to 120 hours.

After completion of the reaction in the step 2-2, the reaction mixture is mixed with water, and then the mixture is extracted with an organic solvent. Thereafter, an organic layer is subjected to post-treatment operations such as washing, drying, and concentration, thereby making it possible to obtain the compound (A6).

The reaction of the step 3-2 is usually performed in a solvent. Examples of the solvent used in the reaction of the step 3-2 include hydrocarbons; ethers; amides; imidazolinones; sulfoxides; secondary alcohols; tertiary alcohols; and mixtures of two or more kinds thereof.

Examples of the base used in the reaction of the step 3-2 include metal alkoxides and metal hydrides.

In the reaction of the step 3-2, the base is usually used in a ratio of 1 to 10 mol with respect to 1 mol of the compound (A6).

The reaction temperature in the step 3-2 is usually in the range of −80 to 150° C. The reaction time in the step 3-2 is usually within the range of 0.1 to 120 hours.

After completion of the reaction in the step 3-2, the reaction mixture is mixed with water, and then the mixture is extracted with an organic solvent. Thereafter, an organic layer is subjected to post-treatment operations such as washing, drying, and concentration, thereby making it possible to obtain the compound (A5).

### Production Method A-5

The compound of the present invention (1-1) can be produced by a step of isocyanating the compound (A5) using an isocyanating agent, then reacting the isocyanated compound with a compound represented by a formula (A7) (hereinafter, referred to as a compound (A7)) and a compound represented by a formula (A8) (hereinafter, referred to as a compound (A8)), in the presence of a base (hereinafter, referred to as a step 4-1).

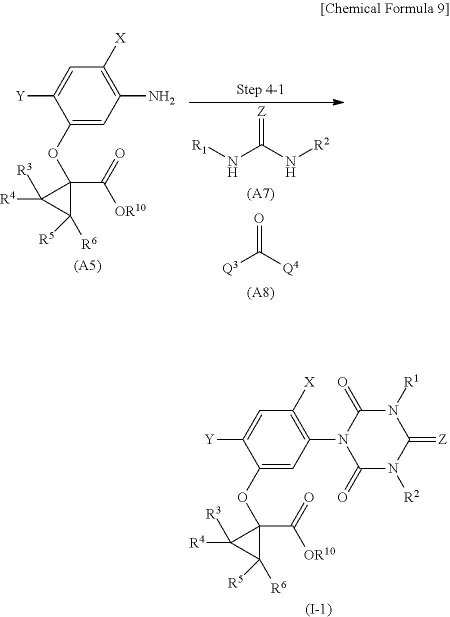

[In the formula, symbols represent the same meaning as described above.]

The reaction of the step 4-1 is usually performed in a solvent. Examples of the solvent used in the reaction of the step 4-1 include hydrocarbons; ethers; halogenated hydrocarbons; amides; esters; nitriles; and mixtures of two or more kinds thereof.

Examples of the isocyanating agent used in the reaction of the step 4-1 include triphosgene (bis(trichloromethyl)carbonate), diphosgene (trichloromethyl chloroformate), and phosgene.

Examples of the base used in the reaction of the step 4-1 include organic bases. Such a base is usually used in a ratio of 1 to 10 mol with respect to 1 mol of the compound (A5).

In the reaction of the step 4-1, when triphosgene is used as an isocyanating agent, the isocyanating agent is usually used in a ratio of 0.3 to 10 mol with respect to 1 mol of the compound (A5). When diphosgene is used as an isocyanating agent, the isocyanating agent is usually used in a ratio of 0.5 to 10 mol with respect to 1 mol of the compound (A5). When phosgene is used as an isocyanating agent, the isocyanating agent is usually used in a ratio of 1 to 10 mol with respect to 1 mol of the compound (A5).

In the reaction of the step 4-1, the compound (A7) is usually used in a ratio of 1 to 10 mol with respect to 1 mol of the compound (A5), and the compound (A8) is usually used in a ratio of 1 to 10 mol with respect to 1 mol of the compound (A5).

The reaction temperature in the step 4-1 is usually in the range of −20 to 150° C. The reaction time in the step 4-1 is usually within the range of 0.1 to 120 hours.

After completion of the reaction in the step 4-1, the reaction mixture is mixed with water, and then the mixture is extracted with an organic solvent. Thereafter, an organic layer is subjected to post-treatment operations such as washing, drying, and concentration, thereby making it possible to obtain the compound of the present invention (I-1).

In the step 4-1, reaction conditions for isocyanation and reaction conditions (solvent, reaction temperature, reaction time, and the like) for a reaction with the compound (A7) and the compound (A8) in the presence of a base after isocyanation (hereinafter, the reaction is referred to as a reaction a) may be the same as or different from each other. The isocyanation and the reaction a may be performed simultaneously by mixing the compound (A5), the isocyanating agent, the base, the compound (A7), and the compound (A8), or the reaction a may be performed after the isocyanation is performed. The reaction a may be started between the start and the completion of the isocyanation. When the reaction a is performed after the isocyanation, the reaction mixture after the isocyanation may be subjected to the reaction a, a mixture obtained by concentrating the reaction mixture after the isocyanation may be subjected to the reaction a, or a product may be isolated from the reaction mixture after the isocyanation, and then the product may be subjected to the reaction a. Such isolation can be performed by mixing the reaction mixture with water, then extracting the mixture with an organic solvent, and causing an organic layer to be subjected to post-treatment operations such as washing, drying, and concentration.

The compound (A7) is a publicly known compound, or can be produced according to a method described in the literature such as Tetrahedron Letters, Vol. 40, No. 10, pages 1957 to 1960 (1999) and a method similar thereto.

The compound (A8) is a publicly known compound, or can be produced according to a method described in the literature such as US 2002/0,065,432 A and a method similar thereto.

### Production Method A-6

The compound of the present invention (1-2) can be produced by a step of reacting the compound of the present invention (I-1) with an acid (hereinafter, referred to as a step 5-1).

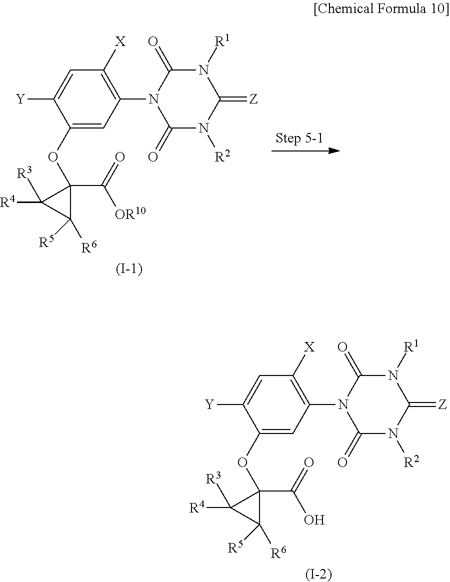

[In the formula, symbols represent the same meaning as described above.]

The reaction of the step 5-1 is usually performed in a solvent. Examples of the solvent used in the reaction of the step 5-1 include ethers; esters; primary alcohol such as methanol (hereinafter, referred to as primary alcohols); secondary alcohols; water; and mixtures of two or more kinds thereof.

Examples of the acid used in the reaction of the step 5-1 include inorganic acids; organic acids; acidic aqueous solution such as iron (III) chloride aqueous solution, iron (III) sulfate aqueous solution, and iron (III) acetylacetonate aqueous solution; and mixtures of two or more kinds thereof.

In the reaction of the step 5-1, the acid is usually used in a ratio of 1 to 100 mol with respect to 1 mol of the compound of the present invention (I-1).

The reaction temperature in the step 5-1 is usually in the range of −20 to 150° C. The reaction time in the step 5-1 is usually within the range of 0.1 to 120 hours.

After completion of the reaction in the step 5-1, the reaction mixture is mixed with water, and then the mixture is extracted with an organic solvent. Thereafter, an organic layer is subjected to post-treatment operations such as washing, drying, and concentration, thereby making it possible to obtain the compound of the present invention (I-2).

### Production Method A-7

The compound represented by the formula (1-3) (hereinafter, referred to as the compound of the present invention (1-3)) can be produced by a step of reacting the compound of the present invention (I-2) with a compound represented by a formula (A9) (hereinafter, referred to as a compound (A9) of the present invention), in the presence of a base (hereinafter, referred to as a step 6-1).

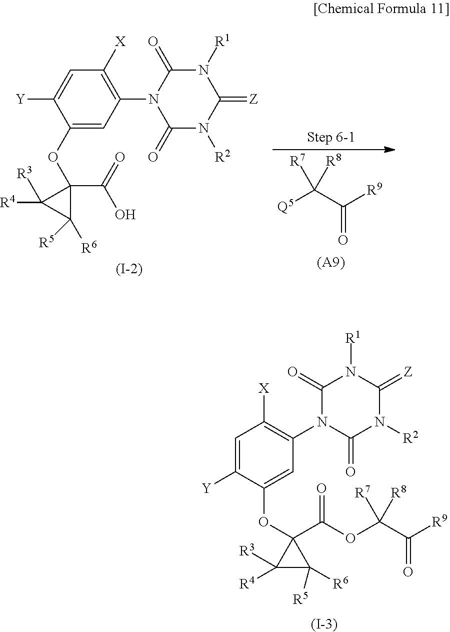

[In the formula, symbols represent the same meaning as described above.]

The reaction of the step 6-1 is usually performed in a solvent. Examples of the solvent used in the reaction of the step 6-1 include hydrocarbons; ethers; halogenated hydrocarbons; amides; imidazolinones; sulfoxides; nitriles; water; and mixtures of two or more kinds thereof.

Examples of the base used in the reaction of the step 6-1 include organic bases; alkali metal carbonates; alkaline earth metal carbonates; alkali metal hydrogen carbonates; metal carboxylates; metal alkoxides; metal hydroxides; metal hydrides, and the like.

In the reaction of the step 6-1, the compound (A9) is usually used in a ratio of 1 to 10 mol, and the base is usually used in a ratio of 1 to 10 mol, with respect to 1 mol of the compound of the present invention (I-2).

The reaction temperature in the step 6-1 is usually in the range of −80 to 150° C. The reaction time in the step 6-1 is usually within the range of 0.1 to 120 hours.

After completion of the reaction in the step 6-1, the reaction mixture is mixed with water, and then the mixture is extracted with an organic solvent. Thereafter, an organic layer is subjected to post-treatment operations such as washing, drying, and concentration, thereby making it possible to obtain the compound of the present invention (1-3).

The compound (A9) is a publicly known compound, or can be produced according to a method described in the literature such as European Journal of Organic Chemistry, vol. 17, pages 3015 to 3023 (2002) and a method similar thereto.

### Production Method A-8

The compound of the present invention (I) can be produced by a step of condensing the compound of the present invention (1-2) and a compound represented by a formula (A10) (hereinafter, referred to as a compound (A10)) in the presence of a condensing agent or in the presence of a condensing agent and a base (hereinafter, referred to as a step 6-2).

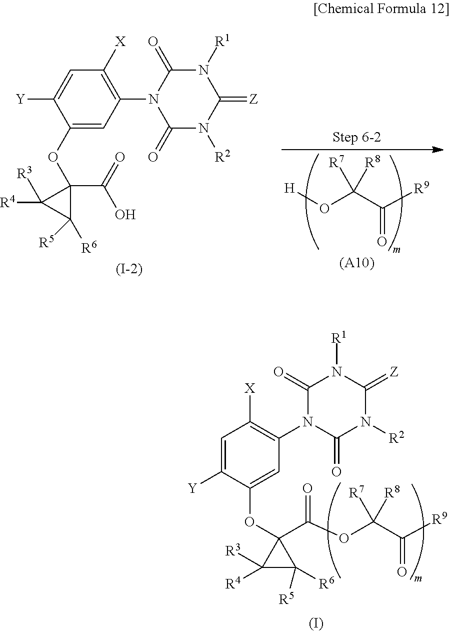

[In the formula, symbols represent the same meaning as described above.]

The reaction of the step 6-2 is usually performed in a solvent. Examples of the solvent used in the reaction of the step 6-2 include hydrocarbons; ethers; halogenated hydrocarbons; amides; imidazolinones; sulfoxides; esters; and mixtures of two or more kinds thereof.

Examples of the condensing agent used in the reaction of the step 6-2 include 1-ethyl-3-(3-dimethylaminopropyl) carbodiimide hydrochloride (hereinafter, referred to as EDCD), dicyclohexylcarbodiimide, benzotriazole-1-yloxytris(dimethylamino)phosphonium hexafluorophosphate, (benzotriazole-1-yloxy)tripyrrolidinophosphonium hexafluorophosphate, 1-[bis(dimethylamino)methylene]-1H-1,2,3-triazolo[4,5-b]pyridinium 3-oxide hexafluorophosphate, and the like. In the reaction of the step 6-2, the condensing agent is usually used in a ratio of 1 to 10 mol with respect to 1 mol of the compound of the present invention (1-2).

Examples of the base that can be used in the reaction of the step 6-2 include alkali metal carbonates, alkaline earth metal carbonates, and organic bases. When the reaction of the step 6-2 is performed in the presence of a base, the base is usually used in a ratio of 1 to 10 mol with respect to 1 mol of the compound of the present invention (I-2).

In the reaction of the step 6-2, the compound (A10) is usually used in a ratio of 1 to 10 mol with respect to 1 mol of the compound of the present invention (I-2).

The reaction temperature in the step 6-2 is usually in the range of −80 to 150° C. The reaction time in the step 6-2 is usually within the range of 0.1 to 120 hours.

After completion of the reaction in the step 6-2, the reaction mixture is mixed with water, and then the mixture is extracted with an organic solvent. Thereafter, an organic layer is subjected to post-treatment operations such as washing, drying, and concentration, thereby making it possible to obtain the compound of the present invention (I).

The compound (A10) is a publicly known compound, or can be produced according to a method described in the literature such as Journal of Organic Chemistry, Vol. 52, No. 22, pages 4978 to 4984 (1987) and the like and a method similar thereto.

### Production Method A-9

The compound of the present invention can also be produced by a step of chlorinating the compound of the present invention (I-2) using a chlorinating agent, then reacting the chlorinated compound of the present invention (I-2) with the compound (A10) in the presence of a base (hereinafter, referred to as a step 6-3).

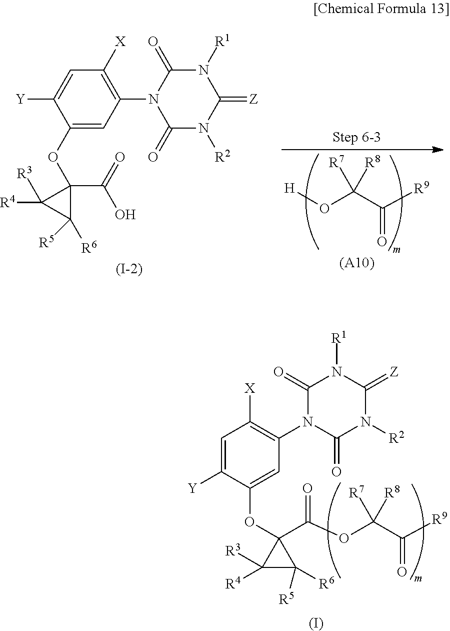

[In the formula, symbols represent the same meaning as described above.]

The reaction of the step 6-3 is usually performed in a solvent. Examples of the solvent used in the reaction of the step 6-3 include hydrocarbons; ethers; halogenated hydrocarbons; and mixtures of two or more kinds thereof.

Examples of the chlorinating agent used in the reaction of the step 6-3 include thionyl chloride, oxalyl chloride, and phosphorus oxychloride. Such a chlorinating agent is usually used in a ratio of 1 to 10 mol with respect to 1 mol of the compound of the present invention (I-2).

Chlorination in the step 6-3 may be performed using a chlorinating agent in the presence of a catalyst. Examples of the catalyst include DMF and the like. Such a catalyst is usually used in a ratio of 0.01 to 1 mol with respect to 1 mol of the compound of the present invention (I-2).

Examples of the base used in the reaction of the step 6-3 include organic bases; alkaline earth metal carbonates; alkali metal carbonates; alkali metal hydrogen carbonates; metal carboxylates; metal hydroxides, and the like. Such a base is usually used in a ratio of 1 to 10 mol with respect to 1 mol of the compound of the present invention (1-2).

The reaction temperature in the step 6-3 is usually in the range of −80 to 150° C. The reaction time in the step 6-3 is usually within the range of 0.1 to 120 hours.

After completion of the reaction in the step 6-3, the reaction mixture is mixed with water, and then the mixture is extracted with an organic solvent. Thereafter, an organic layer is subjected to post-treatment operations such as washing, drying, and concentration, thereby making it possible to obtain the compound of the present invention.

In the step 6-3, reaction conditions for chlorination and reaction conditions (solvent, reaction temperature, reaction time, and the like) for a reaction with the compound (A10) in the presence of a base after chlorination (hereinafter, the reaction is referred to as a reaction b) may be the same as or different from each other. The chlorination and the reaction b may be performed simultaneously by mixing the compound of the present invention (I-2), the chlorinating agent, the base, and the compound (A10), or the reaction b may be performed after chlorination. The reaction b may be started between the start and the completion of chlorination. When the reaction b is performed after chlorination, the reaction mixture after the chlorination may be subjected to the reaction b, a mixture obtained by concentrating the reaction mixture after chlorination may be subjected to the reaction b, or a product may be isolated from the reaction mixture after chlorination and then may be subjected to the reaction b. Such isolation can be performed by mixing the reaction mixture with water, then extracting the mixture with an organic solvent, and causing an organic layer to be subjected to post-treatment operations such as washing, drying, and concentration.

### Production Method B

The compound of the present invention (1-3) can also be produced according to the following scheme.

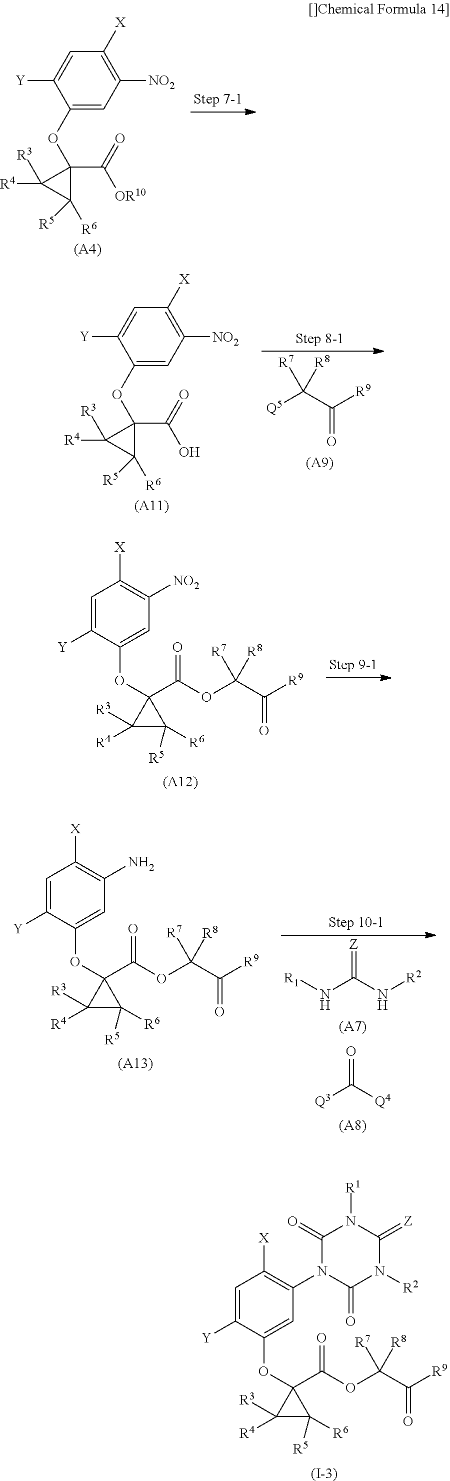

[In the formula, symbols represent the same meaning as described above.]

Hereinafter, each step will be described.

### Production Method B-1

A compound represented by a formula (A11) (hereinafter, referred to as a compound (A11)) can be produced by a step of reacting the compound (A4) with a base (hereinafter, referred to as a step 7-1).

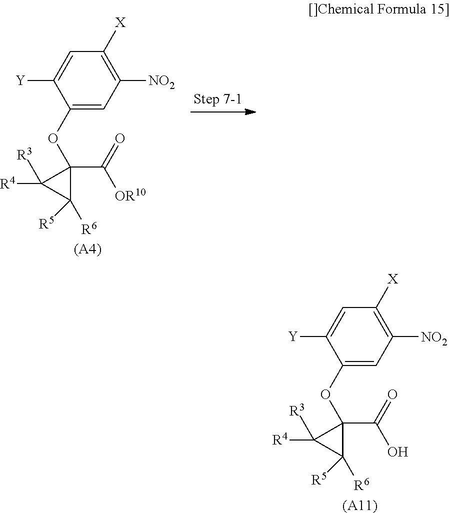

[In the formula, symbols represent the same meaning as described above.]

The reaction of the step 7-1 is usually performed in a solvent. Examples of the solvent used in the reaction of the step 7-1 include ethers; esters; primary alcohols; secondary alcohols; water; and mixtures of two or more kinds thereof.

Examples of the base used in the reaction of the step 7-1 include organic bases; alkali metal carbonates; alkaline earth metal carbonates; alkali metal hydrogen carbonates; metal carboxylates; and metal hydroxides.

In the reaction of the step 7-1, the base is usually used in a ratio of 1 to 10 mol with respect to 1 mol of the compound (A4).

The reaction temperature in the step 7-1 is usually in the range of −80 to 150° C. The reaction time in the step 7-1 is usually within the range of 0.1 to 120 hours.

After completion of the reaction of the step 7-1, the reaction mixture is mixed with water, and the mixture is washed with an organic solvent. Thereafter, an aqueous layer is mixed with an aqueous solution containing an inorganic acid to be acidic, then the mixture is extracted with an organic solvent, and an organic layer is subjected to post-treatment operations such as washing, drying, and concentration, thereby making it possible to obtain the compound (A11).

### Production Method B-2

A compound represented by the formula (A12) (hereinafter, referred to as a compound (A12)) can be produced by a step of reacting the compound (A11) with the compound (A9) in the presence of a base (hereinafter, referred to as a step 8-1).

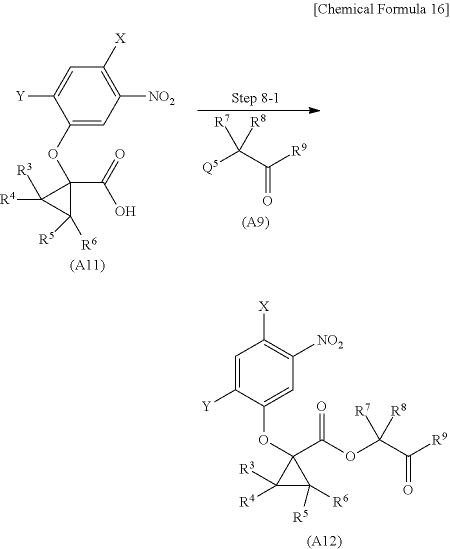

[In the formula, symbols represent the same meaning as described above.]

The reaction of the step 8-1 is usually performed in a solvent. Examples of the solvent used in the reaction of the step 8-1 include hydrocarbons; ethers; halogenated hydrocarbons; amides; imidazolinones; sulfoxides; nitriles; water; and mixtures of two or more kinds thereof.

Examples of the base used in the reaction of the step 8-1 include organic bases; alkali metal carbonates; alkaline earth metal carbonates; alkali metal hydrogen carbonates; metal carboxylates; metal alkoxides; metal hydroxides; metal hydrides, and the like. Such a base is usually used in a ratio of 1 to 10 mol with respect to 1 mol of the compound (A11).

In the reaction of the step 8-1, the compound (A9) is usually used in a ratio of 1 to 10 mol with respect to 1 mol of the compound (A11).

The reaction temperature in the step 8-1 is usually in the range of −80 to 150° C. The reaction time in the step 8-1 is usually within the range of 0.1 to 120 hours.

After completion of the reaction in the step 8-1, the reaction mixture is mixed with water, and then the mixture is extracted with an organic solvent. Thereafter, an organic layer is subjected to post-treatment operations such as washing, drying, and concentration, thereby making it possible to obtain the compound (A12).

### Production Method B-3

A compound represented by a formula (A13) (hereinafter, referred to as a compound (A13)) can be produced by a step of reducing the compound (A12) using a metal such as iron (for example, iron powder), zinc or tin and an acid (hereinafter, referred to as a step 9-1).

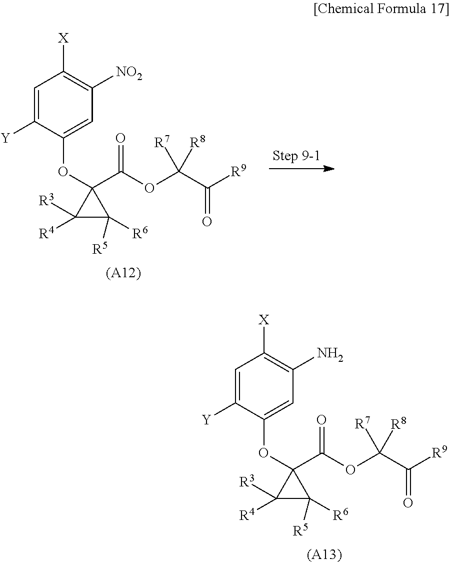

[In the formula, symbols represent the same meaning as described above.]

The reaction of the step 9-1 is usually performed in a solvent. Examples of the solvent used in the reaction of the step 9-1 include esters; hydrocarbons; water; and mixtures of two or more kinds thereof.

Examples of the acid used in the reaction of the step 9-1 include inorganic acids; organic acids; acidic aqueous solution such as iron (III) chloride aqueous solution, iron (III) sulfate aqueous solution, and iron (III) acetylacetonate aqueous solution; and mixtures of two or more kinds thereof.

In the reaction of the step 9-1, the metal is usually used in a ratio of 3 to 100 mol, and the acid is usually used in an excess amount of 1 mol, with respect to 1 mol of the compound (A12).

The reaction temperature in the step 9-1 is usually in the range of −20 to 150° C. The reaction time in the step 9-1 is usually within the range of 0.1 to 120 hours.

After completion of the reaction in the step 9-1, the reaction mixture is mixed with water, and then the mixture is extracted with an organic solvent. Thereafter, an organic layer is subjected to post-treatment operations such as washing, drying, and concentration, thereby making it possible to obtain the compound (A13).

### Production Method B-4

The compound of the present invention (1-3) can be produced by a step of isocyanating the compound (A13) using an isocyanating agent, and then reacting the isocyanated compound with the compound (A7) and the compound (A8) in the presence of a base (hereinafter, referred to as a step 10-1).

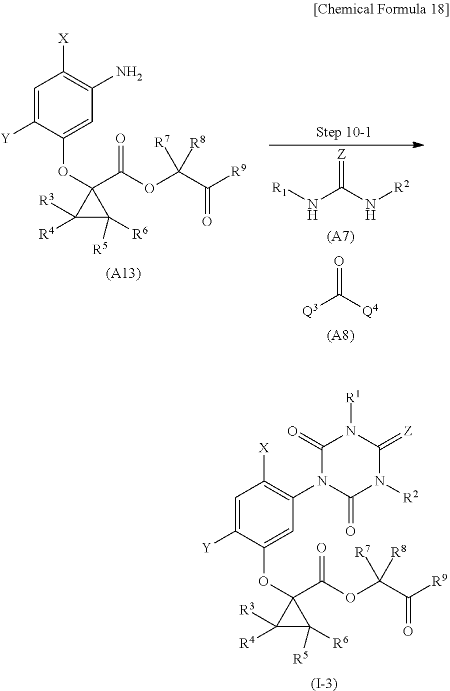

[In the formula, symbols represent the same meaning as described above.]

The reaction of the step 10-1 is usually performed in a solvent. Examples of the solvent used in the reaction of the step 10-1 include hydrocarbons; ethers; halogenated hydrocarbons; amides; esters; nitriles; and mixtures of two or more kinds thereof.

Examples of the isocyanating agent used in the reaction of the step 10-1 include triphosgene (bis(trichloromethyl)carbonate), diphosgene (trichloromethyl chloroformate), and phosgene.

Examples of the base used in the reaction of the step 10-1 include organic bases. Such a base is usually used in a ratio of 1 to 10 mol with respect to 1 mol of the compound (A13).

In the reaction of the step 10-1, when triphosgene is used as an isocyanating agent, triphosgene is usually used in a ratio of 0.3 to 10 mol with respect to 1 mol of the compound (A13). When diphosgene is used as an isocyanating agent, diphosgene is usually used in a ratio of 0.5 to 10 mol with respect to 1 mol of the compound (A13). When phosgene is used as an isocyanating agent, phosgene is usually used in a ratio of 1 to 10 mol with respect to 1 mol of the compound (A13).

In the reaction of the step 10-1, the compound (A7) is usually used in a ratio of 1 to 10 mol with respect to 1 mol of the compound (A13), and the compound (A8) is usually used in a ratio of 1 to 10 mol with respect to 1 mol of the compound (A13).

The reaction temperature in the step 10-1 is usually in the range of −20 to 150° C. The reaction time in the step 10-1 is usually within the range of 0.1 to 120 hours.

After completion of the reaction in the step 10-1, the reaction mixture is mixed with water, and then the mixture is extracted with an organic solvent. Thereafter, an organic layer is subjected to post-treatment operations such as washing, drying, and concentration, thereby making it possible to obtain the compound of the present invention.

In the step 10-1, reaction conditions for isocyanation and reaction conditions (solvent, reaction temperature, reaction time, and the like) for a reaction with the compound (A7) and the compound (A8) in the presence of a base after isocyanation (hereinafter, the reaction is referred to as a reaction c) may be the same as or different from each other. The isocyanation and the reaction c may be performed simultaneously by mixing the compound (A13), the isocyanating agent, the base, the compound (A7), and the compound (A8), or the reaction c may be performed after the isocyanation is performed. The reaction c may be started between the start and the completion of the isocyanation. When the reaction c is performed after the isocyanation, the reaction mixture after the isocyanation may be subjected to the reaction c, a mixture obtained by concentrating the reaction mixture after the isocyanation may be subjected to the reaction c, or a product may be isolated from the reaction mixture after the isocyanation, and then the product may be subjected to the reaction c. Such isolation can be performed by mixing the reaction mixture with water, then extracting the mixture with an organic solvent, and causing an organic layer to be subjected to post-treatment operations such as washing, drying, and concentration.

### Production Method C

The compound of the present invention (1) can also be produced according to the following scheme.

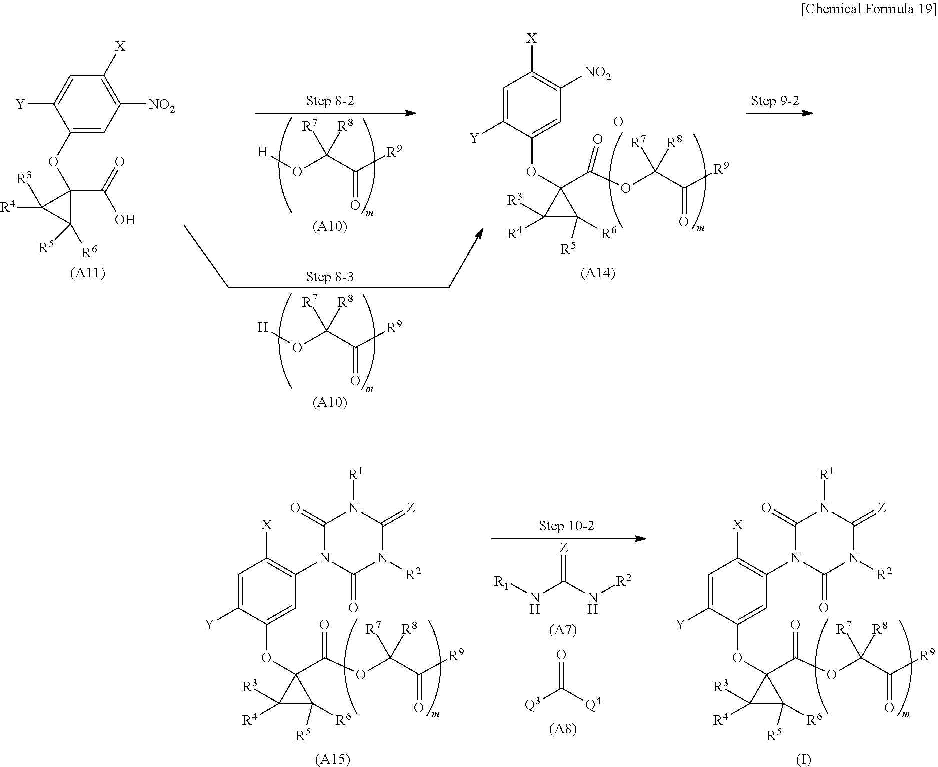

[In the formula, symbols represent the same meaning as described above.]

Hereinafter, each step will be described.

### Production Method C-1

A compound represented by a formula (A14) (hereinafter, referred to as a compound (A14)) can be produced by a step of condensing the compound (A11) and the compound (A10) in the presence of a condensing agent or in the presence of a condensing agent and a base (hereinafter, referred to as a step 8-2).

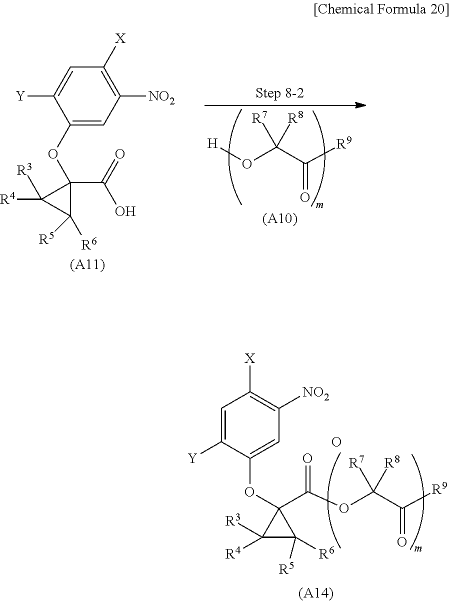

[In the formula, symbols represent the same meaning as described above.]

The reaction of the step 8-2 is usually performed in a solvent. Examples of the solvent used in the reaction of the step 8-2 include hydrocarbons; ethers; halogenated hydrocarbons; amides; imidazolinones; sulfoxides; esters; and mixtures of two or more kinds thereof.

Examples of the condensing agent used in the reaction of the step 8-2 include 1-ethyl-3-(3-dimethylaminopropyl) carbodiimide hydrochloride (hereinafter, referred to as EDCD), dicyclohexylcarbodiimide, benzotriazole-1-yloxytris(dimethylamino)phosphonium hexafluorophosphate, (benzotriazole-1-yloxy)tripyrrolidinophosphonium hexafluorophosphate, 1-[bis(dimethylamino)methylene]-1H-1,2,3-triazolo[4,5-b]pyridinium 3-oxide hexafluorophosphate, and the like. In the reaction of the step 8-2, the condensing agent is usually used in a ratio of 1 to 10 mol with respect to 1 mol of the compound (A11).

Examples of the base that can be used in the reaction of the step 8-2 include alkali metal carbonates, alkaline earth metal carbonates, and organic bases. When the reaction of the step 8-2 is performed in the presence of a base, the base is usually used in a ratio of 1 to 10 mol with respect to 1 mol of the compound (A11).

In the reaction of the step 8-2, the compound (A11) is usually used in a ratio of 1 to 10 mol with respect to 1 mol of the compound (A10).

The reaction temperature in the step 8-2 is usually in the range of −80 to 150° C. The reaction time in the step 8-2 is usually within the range of 0.1 to 120 hours.

After completion of the reaction in the step 8-2, the reaction mixture is mixed with water, and then the mixture is extracted with an organic solvent. Thereafter, an organic layer is subjected to post-treatment operations such as washing, drying, and concentration, thereby making it possible to obtain the compound (A14).

### Production Method C-2

The compound (A14) can also be produced by a step of chlorinating the compound (A11) using a chlorinating agent and then reacting the chlorinated the compound (A11) with the compound (A10) in the presence of a base (hereinafter, referred to as a step 8-3).

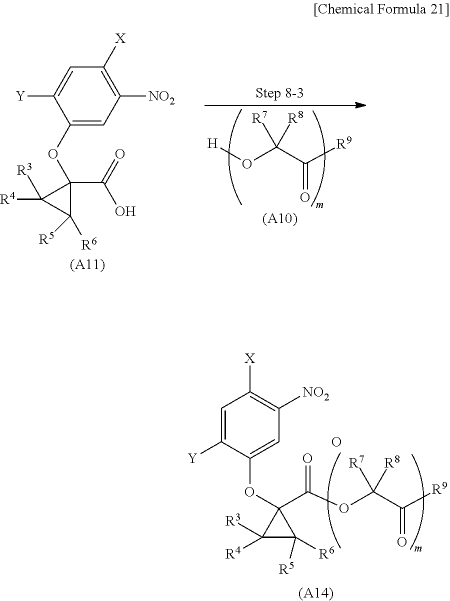

[In the formula, symbols represent the same meaning as described above.]

The reaction of the step 8-3 is usually performed in a solvent. Examples of the solvent used in the reaction of the step 8-3 include hydrocarbons; ethers; halogenated hydrocarbons; and mixtures of two or more kinds thereof.

Examples of the chlorinating agent used in the reaction of the step 8-3 include thionyl chloride, oxalyl chloride, and phosphorus oxychloride. Such a chlorinating agent is usually used in a ratio of 1 to 10 mol with respect to 1 mol of the compound (A11).

Chlorination in the step 8-3 may be performed using a chlorinating agent in the presence of a catalyst. Examples of the catalyst include DMF and the like. Such a catalyst is usually used in a ratio of 0.01 to 1 mol with respect to 1 mol of the compound (A11).

Examples of the base used in the reaction of the step 8-3 include organic bases; alkaline earth metal carbonates; alkali metal carbonates; alkali metal hydrogen carbonates; metal carboxylates; metal hydroxides, and the like. Such a base is usually used in a ratio of 1 to 10 mol with respect to 1 mol of the compound (A11).

The reaction temperature in the step 8-3 is usually in the range of −80 to 150° C. The reaction time in the step 8-3 is usually within the range of 0.1 to 120 hours.

After completion of the reaction in the step 8-3, the reaction mixture is mixed with water, and then the mixture is extracted with an organic solvent. Thereafter, an organic layer is subjected to post-treatment operations such as washing, drying, and concentration, thereby making it possible to obtain the compound of the present invention.

In the step 8-3, reaction conditions for chlorination and reaction conditions (solvent, reaction temperature, reaction time, and the like) for a reaction with the compound (A10) in the presence of a base after chlorination (hereinafter, the reaction is referred to as a reaction d) may be the same as or different from each other. The chlorination and the reaction d may be performed simultaneously by mixing the compound (A11), the chlorinating agent, the base, and the compound (A10), or the reaction d may be performed after the chlorination. The reaction d may be started between the start and the completion of chlorination. When the reaction d is performed after chlorination, a reaction mixture after chlorination may be subjected to the reaction d, a mixture obtained by concentrating the reaction mixture after chlorination may be subjected to the reaction d, or a product may be isolated from the reaction mixture after chlorination and then may be subjected to the reaction d. Such isolation can be performed by mixing the reaction mixture with water, then extracting the mixture with an organic solvent, and causing an organic layer to be subjected to post-treatment operations such as washing, drying, and concentration.

### Production Method C-3

A compound represented by a formula (A15) (hereinafter, referred to as a compound (A15)) can be produced by a step of reducing the compound (A14) using a metal such as iron (for example, iron powder), zinc or tin and an acid (hereinafter, referred to as a step 9-2).

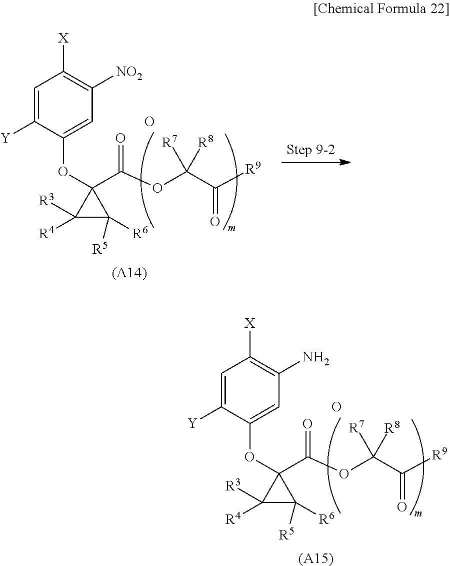

[In the formula, symbols represent the same meaning as described above.]

The reaction of the step 9-2 is usually performed in a solvent. Examples of the solvent used in the reaction of the step 9-2 include esters; hydrocarbons; water; and mixtures of two or more kinds thereof.

Examples of the acid used in the reaction of the step 9-2 include inorganic acids; organic acids; acidic aqueous solution such as iron (III) chloride aqueous solution, iron (III) sulfate aqueous solution, and iron (III) acetylacetonate aqueous solution; and mixtures of two or more kinds thereof.

In the reaction of the step 9-2, the metal is usually used in a ratio of 3 to 100 mol, and the acid is usually used in an excess amount of 1 mol, with respect to 1 mol of the compound (A14).

The reaction temperature in the step 9-2 is usually in the range of −20 to 150° C. The reaction time in the step 9-2 is usually within the range of 0.1 to 120 hours.

After completion of the reaction in the step 9-2, the reaction mixture is mixed with water, and then the mixture is extracted with an organic solvent. Thereafter, an organic layer is subjected to post-treatment operations such as washing, drying, and concentration, thereby making it possible to obtain the compound (A15).

### Production Method C-4

The compound of the present invention (I) can also be produced by a step of isocyanating the compound (A15) using an isocyanating agent, and then reacting the isocyanated compound with the compound (A7) and the compound (A8) in the presence of a base (hereinafter, referred to as a step 10-2).

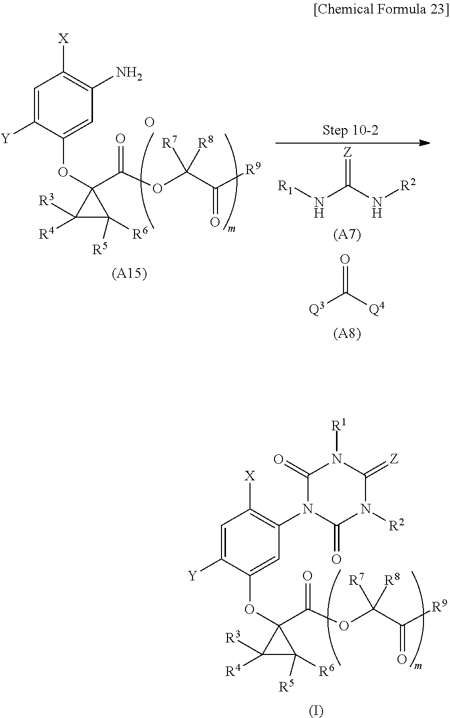

[In the formula, symbols represent the same meaning as described above.]

The reaction of the step 10-2 is usually performed in a solvent. Examples of the solvent used in the reaction of the step 10-2 include hydrocarbons; ethers; halogenated hydrocarbons; amides; esters; nitriles; and mixtures of two or more kinds thereof.

Examples of the isocyanating agent used in the reaction of the step 10-2 include triphosgene (bis(trichlromethyl)carbonate), diphosgene (trichloromethyl chloroformate), and phosgene.

Examples of the base used in the reaction of the step 10-2 include organic bases. Such a base is usually used in a ratio of 1 to 10 mol with respect to 1 mol of the compound (A15).

In the reaction of the step 10-2, when triphosgene is used as an isocyanating agent, triphosgene is usually used in a ratio of 0.3 to 10 mol with respect to 1 mol of the compound (A15). When diphosgene is used as an isocyanating agent, diphosgene is usually used in a ratio of 0.5 to 10 mol with respect to 1 mol of the compound (A15). When phosgene is used as an isocyanating agent, phosgene is usually used in a ratio of 1 to 10 mol with respect to 1 mol of the compound (A15).

In the reaction of the step 10-2, the compound (A7) is usually used in a ratio of 1 to 10 mol with respect to 1 mol of the compound (A15), and the compound (A8) is usually used in a ratio of 1 to 10 mol with respect to 1 mol of the compound (A15).

The reaction temperature in the step 10-2 is usually in the range of −20 to 150° C. The reaction time in the step 10-2 is usually within the range of 0.1 to 120 hours.

After completion of the reaction in the step 10-2, the reaction mixture is mixed with water, and then the mixture is extracted with an organic solvent. Thereafter, an organic layer is subjected to post-treatment operations such as washing, drying, and concentration, thereby making it possible to obtain the compound of the present invention.

In the step 10-2, reaction conditions for isocyanation and reaction conditions (solvent, reaction temperature, reaction time, and the like) for a reaction with the compound (A7) and the compound (A8) in the presence of a base after isocyanation (hereinafter, the reaction is referred to as a reaction e) may be the same as or different from each other. The isocyanation and the reaction e may be performed simultaneously by mixing the compound (A15), the isocyanating agent, the base, the compound (A7), and the compound (A8), or the reaction e may be performed after the isocyanation is performed. The reaction e may be started between the start and the completion of the isocyanation. When the reaction e is performed after the isocyanation, the reaction mixture after the isocyanation may be subjected to the reaction e, a mixture obtained by concentrating the reaction mixture after the isocyanation may be subjected to the reaction e, or a product may be isolated from the reaction mixture after the isocyanation, and then the product may be subjected to the reaction e. Such isolation can be performed by mixing the reaction mixture with water, then extracting the mixture with an organic solvent, and causing an organic layer to be subjected to post-treatment operations such as washing, drying, and concentration.

The compound of the present invention can be mixed or used in combination with one or more components (hereinafter, referred to as a present component) selected from a group consisting of the following group (a), group (b), group (c), group (d), group (e), and group (f).

The term “mixed or used in combination” means that the compound of the present invention and the present component are used simultaneously, separately, or at time intervals.

When the compound of the present invention and the present component are used simultaneously, the compound of the present invention and the present component may be contained in separate formulations or may be contained in one formulation.

One aspect of the present invention is a composition containing one or more components selected from a group consisting of a group (d) and a group (f), and the compound of the present invention.

The group (a) is a group consisting of insecticidal, miticidal, and nematicidal active ingredients and is a group consisting of acetylcholinesterase inhibitors (for example, carbamate insecticides and organophosphorus insecticides), GABAergic chloride channel blockers (for example, phenylpyrazole-based insecticides), sodium channel modulators (for example, pyrethroid insecticides), nicotinic acetylcholine receptor competitive modulators (for example, neonicotinoid insecticides), nicotinic acetylcholine receptor allosteric modulators, glutamatergic chloride channel allosteric modulators (for example, macrolide insecticides), juvenile hormone mimics, multisite inhibitors, chordotonal organ TRPV channel modulators, acaric growth inhibitors, microorganism-derived insect mid-gut disruptors, mitochondrial ATP synthase inhibitors, oxidative phosphorylation uncouplers, nicotinic acetylcholine receptor channel blockers (for example, nereistoxin-based insecticides), chitin biosynthesis inhibitors, molting inhibitors, ecdysone receptor agonists, octopamine receptor agonists, inhibitors of mitochondrial electron transport system complexes I, II, III and IV, voltage-dependent sodium channel blockers, acetyl CoA carboxylase inhibitors, ryanodine receptor modulators (for example, diamide insecticides), chordotonal organ modulators, microbial insecticides, and other insecticidal, miticidal, and nematicidal active ingredients. These are described in the classification based on a mechanism of action of IRAC.

The group (b) is a group of bactericidal active ingredients and is a group consisting of nucleic acid synthesis inhibitors (for example, phenylamide-based microbicides or acylamino acid-based microbicides), cell division and cytoskeleton inhibitors (for example, MBC microbicides), respiratory inhibitors (for example, QoI microbicides, QiI microbicides, SDHI microbicides), amino acid synthesis and protein synthesis inhibitors (for example, anilinopyrimidine-based microbicides), signal transduction inhibitors, lipid synthesis and membrane synthesis inhibitors, sterol biosynthesis inhibitors (for example, DMI microbicides such as a triazole), cell wall biosynthesis inhibitors, melanin synthesis inhibitors, plant defense inducers, multi-action point contact active microbicides, microbial microbicides, and other bactericidal active ingredients. These are described in the classification based on a mechanism of action of FRAC.

The group (c) is a group of plant growth regulators (including mycorrhizae and rhizobia).

The group (d) is a group of phytotoxicity alleviating ingredients.

The group (e) is a group of synergists.

The group (f) is a group of herbicidally active ingredients and is a group consisting of acetyl CoA carboxylase (ACCase) inhibitors, acetolactate synthase (ALS) inhibitors, photosynthesis (photosystem II) inhibitors, photosystem I electron transducers, protoporphyrinogen oxidase (PPO) inhibitors, phytoene desaturase system (PDS) inhibitors, 4-hydroxyphenylpyruvate dioxygenase (4-HPPD) inhibitors, carotenoid biosynthesis inhibitors, EPSP synthase inhibitors, glutamine synthase inhibitors, dihydropteroic acid (DHP) synthase inhibitors, microtubule polymerization inhibitors, mitosis/microtubule formation inhibitors, inhibitors of very long chain fatty acids (VLCFA), cellulose synthesis inhibitors, uncoupling agents, lipid synthesis inhibitors, indoleacetic acid-like actives, auxin transfer inhibitors and other herbicidally active ingredients. These are described in the classification based on a mechanism of action of HRAC.

Hereinafter, examples of combinations of the present component and the compound of the present invention will be described. For example, alanycarb+SX means a combination of alanycarb and SX.

The abbreviation SX means any one compound of the present invention selected from a compound groups SX1 to SX20 described in examples. The present components described below are all known components, and can be obtained from a commercially available formulation or produced by a publicly known method. When the present component is a microorganism, the present component can also be obtained from a bacterial depository. The number in parentheses represents CAS RN (registered trademark).

Combination of the present component from the above group (a) and the compound of the present invention:
abamectin+SX, acephate+SX, acequinocyl+SX, acetamiprid+SX, acetoprole+SX, acrinathrin+SX, acynonapyr+SX, afidopyropen+SX, afoxolaner+SX, alanycarb+SX, aldicarb+SX, allethrin+SX, alpha-cypermethrin+SX, alpha-endosulfan+SX, aluminium phosphide+SX, amitraz+SX, azadirachtin+SX, azamethiphos+SX, azinphos-ethyl+SX, azinphos-methyl+SX, azocyclotin+SX, bark of Celastrus angulatus+SX, bendiocarb+SX, benfluthrin+SX, benfuracarb+SX, bensultap+SX, benzoximate+SX, benzpyrimoxan+SX, beta-cyfluthrin+SX, beta-cypermethrin+SX, bifenazate+SX, bifenthrin+SX, bioallethrin+SX, bioresmethrin+SX, bistrifluron+SX, borax+SX, boric acid+SX, broflanilide+SX, bromopropylate+SX, buprofezin+SX, butocarboxim+SX, butoxycarboxim+SX, cadusafos+SX, calcium phosphide+SX, carbaryl+SX, carbofuran+SX, carbosulfan+SX, cartap hydrochloride+SX, cartap+SX, chinomethionat+SX, chlorantraniliprole+SX, chlordane+SX, chlorethoxyfos+SX, chlorfenapyr+SX, chlorfenvinphos+SX, chlorfluazuron+SX, chlormephos+SX, chloropicrin+SX, chlorpyrifos+SX, chlorpyrifos-methyl+SX, chromafenozide+SX, clofentezine+SX, clothianidin+SX, concanamycin A+SX, coumaphos+SX, cryolite+SX, cyanophos+SX, cyantraniliprole+SX, cyclaniliprole+SX, cyclobutrifluram+SX, cycloprothrin+SX, cycloxaprid+SX, cyenopyrafen+SX, cyetpyrafen+SX, cyflumetofen+SX, cyfluthrin+SX, cyhalodiamide+SX, cyhalothrin+SX, cyhexatin+SX, cypermethrin+SX, cyphenothrin+SX, cyproflanilide+SX, cyromazine+SX, dazomet+SX, deltamethrin+SX, demeton-S-methyl+SX, diafenthiuron+SX, diazinon+SX, dichlorvos+SX, dicloromezotiaz+SX, dicofol+SX, dicrotophos+SX, diflovidazin+SX, diflubenzuron+SX, dimefluthrin+SX, dimethoate+SX, dimethylvinphos+SX, dimpropyridaz+SX, dinotefuran+SX, disodium octaborate+SX, disulfoton+SX, DNOC (2-methyl-4,6-dinitrophenol)+SX, doramectin+SX, dried leaves of Dryopteris filix-mas+SX, emamectin-benzoate+SX, empenthrin+SX, endosulfan+SX, EPN (O-ethyl O-(4-nitrophenyl)phenylphosphonothioate)+SX, epsilon-metofluthrin+SX, epsilon-momfluorothrin+SX, esfenvalerate+SX, ethiofencarb+SX, ethion+SX, ethiprole+SX, ethoprophos+SX, etofenprox+SX, etoxazole+SX, extract of Artemisia absinthium+SX, extract of Azadirachta indica+SX, extract of Cassia nigricans+SX, extract of Clitoria ternatea+SX, extract of Symphytum officinale+SX, extract of Chenopodium ambrosioides+SX, extract of Tanacetum vulgare+SX, extract of Urtica dioica+SX, extract of Viscum album+SX, famphur+SX, fenamiphos+SX, fenazaquin+SX, fenbutatin oxide+SX, fenitrothion+SX, fenmezoditiaz+SX, fenobucarb+SX, fenoxycarb+SX, fenpropathrin+SX, fenpyroximate+SX, fenthion+SX, fenvalerate+SX, fipronil+SX, flometoquin+SX, flonicamid+SX, fluacrypyrim+SX, fluazaindolizine+SX, fluazuron+SX, flubendiamide+SX, fluchlordiniliprole+SX, flucycloxuron+SX, flucythrinate+SX, fluensulfone+SX, flufenoprox+SX, flufenoxuron+SX, flufiprole+SX, flumethrin+SX, flupentiofenox+SX, flupyradifurone+SX, flupyrimin+SX, flupyroxystrobin+SX, fluralaner+SX, fluvalinate+SX, fluxametamide+SX, formetanate+SX, fosthiazate+SX, furamethrin+SX, furathiocarb+SX, gamma-cyhalothrin+SX, GS-omega/kappa HXTX-Hvla peptide+SX, halfenprox+SX, halofenozide+SX, heptafluthrin+SX, heptenophos+SX, hexaflumuron+SX, hexythiazox+SX, potassium salt of hop beta acid+SX, hydramethylnon+SX, hydroprene+SX, imicyafos+SX, imidacloprid+SX, imidaclothiz+SX, imiprothrin+SX, indazapyroxamet+SX, indoxacarb+SX, isocycloseram+SX, isofenphos+SX, isoprocarb SX, isopropyl-O-(methoxyaminothiophosphoryl) salicylate+SX, isoxathion+SX, ivermectin+SX, kadethrin+SX, kappa-tefluthrin+SX, kappa-bifenthrin+SX, kinoprene+SX, lambda-cyhalothrin+SX, ledprona+SX, lenoremycin+SX, lepimectin+SX, lime sulfur+SX, lotilaner+SX, lufenuron+SX, machine oil+SX, malathion+SX, mecarbam+SX, meperfluthrin+SX, metaflumizone+SX, metam+SX, methamidophos+SX, methidathion+SX, methiocarb+SX, methomyl+SX, methoprene+SX, methoxychlor+SX, methoxyfenozide+SX, methyl bromide+SX, metofluthrin+SX, metolcarb+SX, metoxadiazone+SX, mevinphos+SX, milbemectin+SX, milbemycin oxime+SX, mivorilaner+SX, modoflaner+SX, momfluorothrin+SX, monocrotophos+SX, moxidectin+SX, naled+SX, nicofluprole+SX, nicotine+SX, nicotine-sulfate+SX, nitenpyram+SX, novaluron+SX, noviflumuron+SX, oil of the seeds of Chenopodium anthelminticum+SX, omethoate+SX, oxamyl+SX, oxazosulfyl+SX, oxydemeton-methyl+SX, parathion+SX, parathion-methyl+SX, permethrin+SX, phenothrin+SX, phenthoate+SX, phorate+SX, phosalone+SX, phosmet+SX, phosphamidon+SX, phosphine+SX, phoxim+SX, pirimicarb+SX, pirimiphos-methyl+SX, prallethrin+SX, profenofos+SX, profluthrin+SX, propargite+SX, propetamphos+SX, propoxur+SX, propylene glycol alginate+SX, prothiofos+SX, pyflubumide+SX, pymetrozine+SX, pyraclofos+SX, pyrethrins+SX, pyridaben+SX, pyridalyl+SX, pyridaphenthion+SX, pyrifluquinazone+SX, pyrimidifen+SX, pyriminostrobin+SX, pyriprole+SX, pyriproxyfen+SX, quinalphos+SX, resmethrin+SX, rotenone+SX, ryanodine+SX, sarolaner+SX, selamectin+SX, sigma-cypermethrin+SX, silafluofen+SX, sodium borate+SX, sodium metaborate+SX, spidoxamat+SX, spinetoram+SX, spinosad+SX, spirobudifen+SX, spirodiclofen+SX, spiromesifen+SX, spiropidion+SX, spirotetramat+SX, sulfiflumin+SX, sulfluramid+SX, sulfotep+SX, sulfoxaflor+SX, sulfur+SX, sulfuryl fluoride+SX, tartar emetic+SX, tau-fluvalinate+SX, tebufenozide+SX, tebufenpyrad+SX, tebupirimfos+SX, teflubenzuron+SX, tefluthrin+SX, temephos+SX, terbufos+SX, terpene constituents of the extract of Chenopodium ambrosioides near ambrosioides+SX, tetrachlorantraniliprole+SX, tetrachlorvinphos+SX, tetradifon+SX, tetramethrin+SX, tetramethylfluthrin+SX, tetraniliprole+SX, theta-cypermethrin+SX, thiacloprid+SX, thiamethoxam+SX, thiocyclam+SX, thiodicarb+SX, thiofanox+SX, thiometon+SX, thiosultap-disodium+SX, thiosultap-monosodium+SX, tigolaner+SX, tiorantraniliprole+SX, tioxazafen+SX, tolfenpyrad+SX, tralomethrin+SX, transfluthrin+SX, triazamate+SX, triazophos+SX, trichlorfon+SX, trifluenfuronate+SX, triflumezopyrim+SX, triflumuron+SX, trimethacarb+SX, tyclopyrazoflor+SX, umifoxolaner+SX, vamidothion+SX, wood extract of Quassia amara+SX, XMC (3,5-dimethylphenyl N-methylcarbamate)+SX, xylylcarb+SX, zeta-cypermethrin+SX, zinc phosphide+SX, 4-[5-(3,5-dichlorophenyl)-5-(trifluoromethyl)-4,5-dihydro-1,2-oxazol-3-yl]-2-methyl-N-(1-oxothietan-3-yl)benzamide (1241050-20-3)+SX, 3-methoxy-N-(5-{5-(trifluoromethyl)-5-[3-(trifluoromethyl)phenyl]-4,5-dihydro-1,2-oxazol-3-yl}indan-1-yl)propanamide (1118626-57-5)+SX, N-{4-chloro-3-[(1-cyanocyclopropyl)carbamoyl]phenyl}-1-methyl-4-(methanesulfonyl)-3-(1,1,2,2,2-pentafluoroethyl)-1H-pyrazole-3-carboxamide (1400768-21-9)+SX, N-[3-chloro-1-(pyridin-3-yl)-1H-pyrazol-4-yl]-2-(methanesulfonyl)propanamide (2396747-83-2)+SX, N-[4-chloro-2-(pyridin-3-yl)-1,3-thiazol-5-yl]-N-ethyl-3-(methanesulfonyl)propanamide+SX, 1,4-dimethyl-2-[2-(pyridin-3-yl)-2H-indazol-5-yl]-1,2,4-triazolidine-3,5-dione (2171099-09-3)+SX, 2-isopropyl-5-[(3,4,4-trifluoro-3-buten-1-yl)sulfonyl]-1,3,4-thiadiazole (2058052-95-0)+SX, N-({2-fluoro-4-[(2S,3S)-2-hydroxy-3-(3,4,5-trichlorophenyl)-3-(trifluoromethyl)pyrrolidin-1-yl]phenyl}methyl)cyclopropanecarboxamide+SX, 7-fluoro-N-[1-(methylsulfanyl)-2-methylpropan-2-yl]-2-(pyridin-3-yl)-2H-indazole-4-carboxamide+SX, 7-fluoro-N-[1-(methanesulfinyl)-2-methylpropan-2-yl]-2-(pyridin-3-yl)-2H-indazole-4-carboxamide+SX, 7-fluoro-N-[1-(methanesulfonyl)-2-methylpropan-2-yl]-2-(pyridin-3-yl)-2H-indazole-4-carboxamide+SX, N-[1-(difluoromethyl)cyclopropyl]-2-(pyridin-3-yl)-2H-indazole-4-carboxamide+SX, 2,9-dihydro-9-(methoxymethyl)-2-(pyridin-3-yl)-10H-pyrazolo[3,4-f]pyrido[2,3-b][1,4]oxazepin-10-one (2607927-97-7)+SX, BT crop protein Cry1Ab+SX, BT crop protein Cry1Ac+SX, BT crop protein Cry1Fa+SX, BT crop protein Cry1A.105+SX, BT crop protein Cry2Ab+SX, BT crop protein Vip3A+SX, BT crop protein mCry3A+SX, BT crop protein Cry3Ab+SX, BT crop protein Cry3Bb+SX, BT crop protein Cry34Ab1/Cry35Ab1+SX, Adoxophyes orana GV (granulovirus) strain BV-0001+SX, Anticarsia gemmatalis MNPV (multiple nucleocapsid nucleopolyhedrovirus)+SX, Autographa californica MNPV+SX, Cydia pomonella GV strain V15+SX, Cydia pomonella GV strain V22+SX, Cryptophlebia leucotreta GV+SX, Dendrolimus punctatus cypovirus+SX, Helicoverpa armigera NPV (nucleopolyhedrovirus) strain BV-0003+SX, Helicoverpa zea NPV+SX, Lymantria dispar NPV+SX, Mamestra brassicae NPV+SX, Mamestra configurata NPV+SX, Neodiprion abietis NPV+SX, Neodiprion lecontei NPV+SX, Neodiprion sertifer NPV+SX, Nosema locustae+SX, Orgyia pseudotsugata NPV+SX, Pieris rapae GV+SX, Plodia interpunctella GV+SX, Spodoptera exigua MNPV+SX, Spodoptera littoralis MNPV+SX, Spodoptera litura NPV+SX, Arthrobotrys dactyloides+SX, Bacillus firmus strain GB-126+SX, Bacillus firmus strain I-1582+SX, Bacillus firmus strain NCIM2637+SX, Bacillus megaterium+SX, Bacillus sp. strain AQ175+SX, Bacillus sp. strain AQ177+SX, Bacillus sp. strain AQ178+SX, Bacillus sphaericus strain 2362 serotype H5a5b+SX, Bacillus sphaericus strain ABTS1743+SX, Bacillus thuringiensis strain AQ52+SX, Bacillus thuringiensis strain BD #32+SX, Bacillus thuringiensis strain CR-371+SX, Bacillus thuringiensis subsp. Aizawai strain ABTS-1857+SX, Bacillus thuringiensis subsp. Aizawai strain AM65-52+SX, Bacillus thuringiensis subsp. Aizawai strain GC-91+SX, Bacillus thuringiensis subsp. Aizawai strain NB200+SX, Bacillus thuringiensis subsp. Aizawai Serotype strain H-7+SX, Bacillus thuringiensis subsp. Kurstaki strain ABTS351+SX, Bacillus thuringiensis subsp. Kurstaki strain BMP123+SX, Bacillus thuringiensis subsp. Kurstaki strain CCT1306+SX, Bacillus thuringiensis subsp. Kurstaki strain EG2348+SX, Bacillus thuringiensis subsp. Kurstaki strain EG7841+SX, Bacillus thuringiensis subsp. Kurstaki strain EVB113-19+SX, Bacillus thuringiensis subsp. Kurstaki strain F810+SX, Bacillus thuringiensis subsp. Kurstaki strain HD-1+SX, Bacillus thuringiensis subsp. Kurstaki strain PB54+SX, Bacillus thuringiensis subsp. Kurstaki strain SA-11+SX, Bacillus thuringiensis subsp. Kurstaki strain SA-12+SX, Bacillus thuringiensis subsp. Tenebriosis strain NB176+SX, Bacillus thuringiensis subsp. Thuringiensis strain MPPL002+SX, Bacillus thuringiensis subsp. morrisoni+SX, Bacillus thuringiensis var. colmeri+SX, Bacillus thuringiensis var. darmstadiensis strain 24-91+SX, Bacillus thuringiensis var. dendrolimus+SX, Bacillus thuringiensis var. galleriae+SX, Bacillus thuringiensis var. israelensis strain BMP144+SX, Bacillus thuringiensis var. israelensis (serotype H-14) strain+SX, Bacillus thuringiensis var. japonensis strain buibui+SX, Bacillus thuringiensis var. san diego strain M-7+SX, Bacillus thuringiensis var. 7216+SX, Bacillus thuringiensis var. aegypti+SX, Bacillus thuringiensis var. T36+SX, Beauveria bassiana strain ANT-03+SX, Beauveria bassiana strain ATCC74040+SX, Beauveria bassiana strain GHA+SX, Beauveria brongniartii+SX, Burkholderia rinojensis strain A396+SX, Chromobacterium subtsugae strain PRAA4-1T+SX, Dactyllela ellipsospora+SX, Dectylaria thaumasia+SX, Hirsutella minnesotensis+SX, Hirsutella rhossiliensis+SX, Hirsutella thompsonii+SX, Lagenidium giganteum+SX, Lecanicillium lecanii strain KV01+SX, Lecanicillium lecanii conidia of strain DAOM198499+SX, Lecanicillium lecanii conidia of strain DAOM216596+SX, Lecanicillium muscarium strain Ve6+SX, Metarhizium anisopliae strain F52+SX, Metarhizium anisopliae var. acridum+SX, Metarhizium anisopliae var. anisopliae BIPESCO 5/F52+SX, Metarhizium flavoviride+SX, Monacrosporium phymatopagum+SX, Paecilomyces fumosoroseus Apopka strain 97+SX, Paecilomyces lilacinus strain 251+SX, Paecilomyces tenuipes strain T1+SX, Paenibacillus popilliae+SX, Pasteuria nishizawae strain Pn1+SX, Pasteuria penetrans+SX, Pasteuria usgae+SX, Pasteuria thornei+SX, Serratia entomophila+SX, Verticillium chlamydosporium+SX, Verticillium lecani strain NCIM1312+SX, and Wolbachia pipientis+SX.

Combination of the present component from the above group (b) and the compound of the present invention:
acibenzolar-S-methyl+SX, aldimorph+SX, ametoctradin+SX, aminopyrifen+SX, amisulbrom+SX, anilazine+SX, azaconazole+SX, azoxystrobin+SX, basic copper sulfate+SX, benalaxyl+SX, benalaxyl-M+SX, benodanil+SX, benomyl+SX, benthiavalicarb+SX, benthiavalicarb-isopropyl+SX, benzovindiflupyr+SX, binapacryl+SX, biphenyl+SX, bitertanol+SX, bixafen+SX, blasticidin-S+SX, Bordeaux mixture+SX, boscalid+SX, bromothalonil+SX, bromuconazole+SX, bupirimate+SX, captafol+SX, captan+SX, carbendazim+SX, carboxin+SX, carpropamid+SX, chinomethionat+SX, chitin+SX, chloroinconazide+SX, chloroneb+SX, chlorothalonil+SX, chlozolinate+SX, colletochlorin B+SX, copper (II) acetate+SX, copper (II) hydroxide+SX, copper oxychloride+SX, copper (II) sulfate+SX, coumoxystrobin+SX, cyazofamid+SX, cyflufenamid+SX, cymoxanil+SX, cyproconazole+SX, cyprodinil+SX, dichlobentiazox+SX, dichlofluanid+SX, diclocymet+SX, diclomezine+SX, dicloran+SX, diethofencarb+SX, difenoconazole+SX, diflumetorim+SX, dimethachlone+SX, dimethirimol+SX, dimethomorph+SX, dimoxystrobin+SX, diniconazole+SX, diniconazole-M+SX, dinocap+SX, dipotassium hydrogenphosphite+SX, dipymetitrone+SX, dithianon+SX, dodecylbenzenesulphonic acid bisethylenediamine copper (II) salt+SX, dodemorph+SX, dodine+SX, edifenphos+SX, enoxastrobin+SX, epoxiconazole+SX, etaconazole+SX, ethaboxam+SX, ethirimol+SX, etridiazole+SX, extract of Melaleuca alternifolia+SX, extract of Reynoutria sachalinensis+SX, extract of the cotyledons of lupine plantlets (“BLAD”)+SX, extract of Allium sativum+SX, extract of Equisetum arvense+SX, extract of Tropaeolum majus+SX, famoxadone+SX, fenamidone+SX, fenaminstrobin+SX, fenarimol+SX, fenbuconazole+SX, fenfuram+SX, fenhexamid+SX, fenoxanil+SX, fenpiclonil+SX, fenpicoxamid+SX, fenpropidin+SX, fenpropimorph+SX, fenpyrazamine+SX, fentin acetate+SX, fentin chloride+SX, fentin hydroxide+SX, ferbam+SX, ferimzone+SX, florylpicoxamid+SX, fluazinam+SX, flubeneteram+SX, fludioxonil+SX, flufenoxadiazam+SX, flufenoxystrobin+SX, fluindapyr+SX, flumetylsulforim+SX, flumorph+SX, fluopicolide+SX, fluopyram+SX, fluopimomide+SX, fluoroimide+SX, fluoxapiprolin+SX, fluoxastrobin+SX, fluoxytioconazole+SX, fluquinconazole+SX, flusilazole+SX, flusulfamide+SX, flutianil+SX, flutolanil+SX, flutriafol+SX, fluxapyroxad+SX, folpet+SX, fosetyl+SX, fosetyl-aluminium+SX, fuberidazole+SX, furalaxyl+SX, furametpyr+SX, guazatine+SX, hexaconazole+SX, hymexazole+SX, imazalil+SX, imibenconazole+SX, iminoctadine+SX, iminoctadine triacetate+SX, inpyrfluxam+SX, iodocarb+SX, ipconazole+SX, ipfentrifluconazole+SX, ipflufenoquin+SX, iprobenfos+SX, iprodione+SX, iprovalicarb+SX, isofetamid+SX, isoflucypram+SX, isoprothiolane+SX, isopyrazam+SX, isotianil+SX, kasugamycin+SX, kresoxim-methyl+SX, laminarin+SX, leaves and bark of Quercus+SX, mancozeb+SX, mandestrobin+SX, mandipropamid+SX, maneb+SX, mefentrifluconazole+SX, mepanipyrim+SX, mepronil+SX, meptyldinocap+SX, metalaxyl+SX, metalaxyl-M+SX, metarylpicoxamid+SX, metconazole+SX, methasulfocarb+SX, metiram+SX, metominostrobin+SX, metrafenone+SX, metyltetraprole+SX, myclobutanil+SX, naftifine+SX, nuarimol+SX, octhilinone+SX, ofurace+SX, orysastrobin+SX, oxadixyl+SX, oxathiapiprolin+SX, oxine-copper+SX, oxolinic acid+SX, oxpoconazole+SX, oxpoconazole fumarate+SX, oxycarboxin+SX, oxytetracycline+SX, pefurazoate+SX, penconazole+SX, pencycuron+SX, penflufen+SX, penthiopyrad+SX, phenamacril+SX, phosphorous acid+SX, phthalide+SX, picarbutrazox+SX, picoxystrobin+SX, piperalin+SX, polyoxins+SX, potassium hydrogencarbonate+SX, potassium dihydrogenphosphite+SX, probenazole+SX, prochloraz+SX, procymidone+SX, propamidine+SX, propamocarb+SX, propiconazole+SX, propineb+SX, proquinazid+SX, prothiocarb+SX, prothioconazole+SX, pydiflumetofen+SX, pyraclostrobin+SX, pyrametostrobin+SX, pyraoxystrobin+SX, pyrapropoyne+SX, pyraziflumid+SX, pyrazophos+SX, pyribencarb+SX, pyributicarb+SX, pyridachlometyl+SX, pyrifenox+SX, pyrimethanil+SX, pyrimorph+SX, pyriofenone+SX, pyrisoxazole+SX, pyroquilon+SX, Quillaja extract+SX, quinconazole+SX, quinofumelin+SX, quinoxyfen+SX, quintozene+SX, Saponins of Chenopodium quinoa+SX, seboctylamine+SX, sedaxane+SX, silthiofam+SX, simeconazole+SX, sodium hydrogencarbonate+SX, spiroxamine+SX, streptomycin+SX, sulfur+SX, tebuconazole+SX, tebufloquin+SX, teclofthalam+SX, tecnazene+SX, terbinafine+SX, tetraconazole+SX, thiabendazole+SX, thifluzamide+SX, thiophanate+SX, thiophanate-methyl+SX, thiram+SX, thymol+SX, tiadinil+SX, tolclofos-methyl+SX, tolfenpyrad+SX, tolprocarb+SX, tolylfluanid+SX, triadimefon+SX, triadimenol+SX, triazoxide+SX, triclopyricarb+SX, tricyclazole+SX, tridemorph+SX, trifloxystrobin+SX, triflumizole+SX, triforine+SX, triticonazole+SX, validamycin+SX, valifenalate+SX, vinclozolin+SX, yellow mustard powder+SX, zinc thiazole+SX, zineb+SX, ziram+SX, zoxamide+SX, N′-[4-({3-[(4-chlorophenyl)methyl]-1,2,4-thiadiazol-5-yl}oxy)-2,5-dimethylphenyl]-N-ethyl-N-methylmethanimidamide (1202781-91-6)+SX, N′-{4-[(4,5-dichlorothiazol-2-yl)oxy]-2,5-dimethylphenyl}-N-ethyl-N-methylmethanimidamide (929908-57-6)+SX, N′-(2,5-dimethyl-4-phenoxyphenyl)-N-ethyl-N-methylmethanimidamide (1052688-31-9)+SX, N′-[5-chloro-4-(2-fluorophenoxy)-2-methylphenyl]-N-ethyl-N-methylmethanimidamide (2055589-28-9)+SX, N′-[2-chloro-4-(2-fluorophenoxy)-5-methylphenyl]-N-ethyl-N-methylmethanimidamide (2055756-21-1)+SX, N′-(2-chloro-4-phenoxy-5-methylphenyl)-N-ethyl-N-methylmethanimidamide (2062599-39-5)+SX, N′-[4-(1-hydroxy-1-phenyl-2,2,2-trifluoroethyl)-2-methyl-5-methoxyphenyl]-N-isopropyl-N-methylmethanimidamide (2101814-55-3)+SX, N′-[5-bromo-6-(1-methyl-2-propoxyethoxy)-2-methylpyridin-3-yl]-N-ethyl-N-methylmethanimidamide (1817828-69-5)+SX, 4-(2-bromo-4-fluorophenyl)-N-(2-chloro-6-fluorophenyl)-1,3-dimethyl-1H-pyrazol-5-amine (1362477-26-6)+SX, 2-[6-(3-fluoro-4-methoxyphenyl)-5-methylpyridin-2-yl]quinazoline (1257056-97-5)+SX, ethyl (2Z)-3-amino-2-cyano-3-phenylacrylate (39491-78-6)+SX, N-[(2-chlorothiazol-5-yl)methyl]-N-ethyl-6-methoxy-3-nitropyridin-2-amine (1446247-98-8)+SX, 5-(4-chlorobenzyl)-2-(chloromethyl)-2-methyl-1-(1H-1,2,4-triazol-1-ylmethyl)cyclopentan-1-ol (1394057-11-4)+SX, (1R,2S, 5S)-5-(4-chlorobenzyl)-2-(chloromethyl)-2-methyl-1-(1H-1,2,4-triazol-1-ylmethyl)cyclopentan-1-ol (1801930-06-2)+SX, (1S,2R, 5R)-5-(4-chlorobenzyl)-2-(chloromethyl)-2-methyl-1-(1H-1,2,4-triazol-1-ylmethyl)cyclopentan-1-ol (1801930-07-3)+SX, 2-(chloromethyl)-5-(4-fluorobenzyl)-2-methyl-1-(1H-1,2,4-triazol-1-ylmethyl)cyclopentan-1-ol (1394057-13-6)+SX, (1R,2S, 5S)-2-(chloromethyl)-5-(4-fluorobenzyl)-2-methyl-1-(1H-1,2,4-triazol-1-ylmethyl)cyclopentan-1-ol (1801930-08-4)+SX, (1S,2R, 5R)-2-(chloromethyl)-5-(4-fluorobenzyl)-2-methyl-1-(1H-1,2,4-triazol-1-ylmethyl)cyclopentan-1-ol (1801930-09-5)+SX, methyl 3-[(4-chlorophenyl)methyl]-2-hydroxy-1-methyl-2-(1H-1,2,4-triazol-1-ylmethyl)cyclopentan-1-carboxylate (1791398-02-1)+SX, 1-(2,4-difluorophenyl)-2-(1H-1,2,4-triazol-1-yl)-1-[1-(4-bromo-2,6-difluorophenoxy)cyclopropyl]ethanol (2019215-86-0)+SX, 1-(2,4-difluorophenyl)-2-(1H-1,2,4-triazol-1-yl)-1-[1-(4-chloro-2,6-difluorophenoxy)cyclopropyl]ethanol (2019215-84-8)+SX, 1-[2-(1-chlorocyclopropyl)-3-(2-fluorophenyl)-2-hydroxypropyl]-1H-imidazole-5-carbonitrile (2018316-13-5)-+SX, 1-[2-(1-chlorocyclopropyl)-3-(2,3-difluorophenyl)-2-hydroxypropyl]-1H-imidazole-5-carbonitrile (2018317-25-2)+SX, 2-[6-(4-bromophenoxy)-2-(trifluoromethyl)pyridin-3-yl]-1-(1H-1,2,4-triazol-1-yl)propan-2-ol (2082661-43-4) SX, 2-[6-(4-chlorophenoxy)-2-(trifluoromethyl)pyridin-3-yl]-1-(1H-1,2,4-triazol-1-yl)propan-2-ol (2082660-27-1)+SX, methyl ({2-methyl-5-[1-(4-methoxy-2-methylphenyl)-1H-pyrazol-3-yl]phenyl}methyl)carbamate (1605879-98-8)+SX, 2-(difluoromethyl)-N-[1,1,3-trimethyl-2,3-dihydro-1H-inden-4-yl]pyridine-3-carboxamide (1616239-21-4)+SX, 2-(difluoromethyl)-N-[3-ethyl-1,1-dimethyl-2,3-dihydro-1H-inden-4-yl]pyridine-3-carboxamide (1847460-02-9)+SX, 2-(difluoromethyl)-N-[3-propyl-1,1-dimethyl-2,3-dihydro-1H-inden-4-yl]pyridine-3-carboxamide (1847460 May 2)+SX, (2E,3Z)-5-{[1-(4-chlorophenyl)-1H-pyrazol-3-yl]oxy}-2-(methoxyimino)-N,3-dimethylpent-3-enamide (1445331-27-0)+SX, (2E,3Z)-5-{[1-(2,4-dichlorophenyl)-1H-pyrazol-3-yl]oxy}-2-(methoxyimino)-N,3-dimethylpent-3-enamide (1445331-54-3)+SX, 5-chloro-4-({2-[6-(4-chlorophenoxy)pyridin-3-yl]ethyl}amino)-6-methylpyrimidine (1605340-92-8)+SX, N-(1-benzyl-1,3-dimethylbutyl)-8-fluoroquinoline-3-carboxamide (2132414-04-9)+SX, N-(1-benzyl-3,3,3-trifluoro-1-methylpropyl)-8-fluoroquinoline-3-carboxamide (2132414-00-5)+SX, 4,4-dimethyl-2-({4-[5-(trifluoromethyl)-1,2,4-oxadiazol-3-yl]phenyl}methyl)isoxazolidin-3-one (2098918-25-1)+SX, 5,5-dimethyl-2-({4-[5-(trifluoromethyl)-1,2,4-oxadiazol-3-yl]phenyl}methyl)isoxazolidin-3-one (2098918-26-2)+SX, N-ethyl-2-methyl-N-({4-[5-(trifluoromethyl)-1,2,4-oxadiazol-3-yl]phenyl}methyl)propanamide+SX, N,2-dimethoxy-N-({4-[5-(trifluoromethyl)-1,2,4-oxadiazol-3-yl]phenyl}methyl)propanamide+SX, N-methoxy-N-({4-[5-(trifluoromethyl)-1,2,4-oxadiazol-3-yl]phenyl}methyl)cyclopropanecarboxamide+SX, N-methoxy-N′-methyl-N-({4-[5-(trifluoromethyl)-1,2,4-oxadiazol-3-yl]phenyl}methyl)urea+SX, N′-ethyl-N-methoxy-N-({4-[5-(trifluoromethyl)-1,2,4-oxadiazol-3-yl]phenyl}methyl)urea+SX, N,N′-dimethoxy-N-({4-[5-(trifluoromethyl)-1,2,4-oxadiazol-3-yl]phenyl}methyl)urea+SX, N-acetyl-2-(ethanesulfonyl)-N-[2-(methoxycarbonyl)-4-(trifluoromethoxy)phenyl]-4-(trifluoromethyl)benzamide (2043675-28-9)+SX, 3-(4-bromo-7-fluoroindol-1-yl)butan-2-yl N-[(3-hydroxy-4-methoxypyridin-2-yl)carbonyl]-L-alaninate+SX, 3-(7-bromoindol-1-yl)butan-2-yl N-[(3-hydroxy-4-methoxypyridin-2-yl)carbonyl]-L-alaninate+SX, 3-(7-bromo-4-fluoroindol-1-yl)butan-2-yl N-[(3-hydroxy-4-methoxypyridin-2-yl)carbonyl]-L-alaninate+SX, 3-(3,5-dichloropyridin-2-yl)butan-2-yl N-[(3-hydroxy-4-methoxypyridin-2-yl)carbonyl]-L-alaninate+SX, 3-(3,5-dichloropyridin-2-yl)butan-2-yl N-{[3-(acetoxymethoxy)-4-methoxypyridin-2-yl]carbonyl}-L-alaninate 1 SX, (1S)-1-[1-(naphthalen-1-yl)cyclopropyl]ethyl N-[(3-hydroxy-4-methoxypyridin-2-yl)carbonyl]-L-alaninate+SX, (1S)-1-[1-(naphthalen-1-yl)cyclopropyl]ethyl N-[(3-acetoxy-4-methoxypyridin-2-yl)carbonyl]-L-alaninate+SX, (1S)-1-[1-(naphthalen-1-yl)cyclopropyl]ethyl N-{[3-(acetoxymethoxy)-4-methoxypyridin-2-yl]carbonyl}-L-alaninate+SX, N-({4-[5-(trifluoromethyl)-1,2,4-oxadiazol-3-yl]phenyl}methyl)cyclopropanecarboxamide+SX, N-allyl-N-({4-[5-(trifluoromethyl)-1,2,4-oxadiazol-3-yl]phenyl}methyl)acetamide+SX, N-allyl-N-({4-[5-(trifluoromethyl)-1,2,4-oxadiazol-3-yl]phenyl}methyl)propanamide+SX, N-({4-[5-(trifluoromethyl)-1,2,4-oxadiazol-3-yl]phenyl}methyl)propanamide+SX, 3,3,3-trifluoro-N-({2-fluoro-4-[5-(trifluoromethyl)-1,2,4-oxadiazol-3-yl]phenyl}methyl)propanamide+SX, 3,3,3-trifluoro-N-({3-fluoro-4-[5-(trifluoromethyl)-1,2,4-oxadiazol-3-yl]phenyl}methyl)propanamide+SX, 3,3,3-trifluoro-N-({2,3-difluoro-4-[5-(trifluoromethyl)-1,2,4-oxadiazol-3-yl]phenyl}methyl)propanamide+SX, N-({2,3-difluoro-4-[5-(trifluoromethyl)-1,2,4-oxadiazol-3-yl]phenyl}methyl)butanamide+SX, N-methoxy-N-methyl-N′-({4-[5-(trifluoromethyl)-1,2,4-oxadiazol-3-yl]phenyl}methyl)urea SX, N, N-diethyl-N′-({4-[5-(trifluoromethyl)-1,2,4-oxadiazol-3-yl]phenyl}methyl)urea+SX, N-methyl-N′-({4-[5-(trifluoromethyl)-1,2,4-oxadiazol-3-yl]phenyl}methyl)urea+SX, 1-({4-[5-(trifluoromethyl)-1,2,4-oxadiazol-3-yl]phenyl}methyl)pyrrolidin-2-one+SX, 1-({4-[5-(trifluoromethyl)-1,2,4-oxadiazol-3-yl]phenyl}methyl) piperidin-2-one+SX, 4-({4-[5-(trifluoromethyl)-1,2,4-oxadiazol-3-yl]phenyl}methyl) morpholin-3-one+SX, 2-({4-[5-(trifluoromethyl)-1,2,4-oxadiazol-3-yl]phenyl}methyl)isoxazolidin-3-one+SX, 3,3-dimethyl-1-({4-[5-(trifluoromethyl)-1,2,4-oxadiazol-3-yl]phenyl}methyl) piperidin-2-one+SX, 2-({4-[5-(trifluoromethyl)-1,2,4-oxadiazol-3-yl]phenyl}methyl)-1,2-oxazinan-3-one+SX, 1-({3-fluoro-4-[5-(trifluoromethyl)-1,2,4-oxadiazol-3-yl]phenyl}methyl) azepan-2-one+SX, 4,4-dimethyl-1-({4-[5-(trifluoromethyl)-1,2,4-oxadiazol-3-yl]phenyl}methyl)pyrrolidin-2-one+SX, 5-methyl-1-({4-[5-(trifluoromethyl)-1,2,4-oxadiazol-3-yl]phenyl}methyl)pyrrolidin-2-one+SX, ethyl 1-({4-[5-(trifluoromethyl)-1,2,4-oxadiazol-3-yl]phenyl}methyl)-1H-pyrazole-4-carboxylate+SX, N-methyl-1-({4-[5-(trifluoromethyl)-1,2,4-oxadiazol-3-yl]phenyl}methyl)-1H-pyrazole-4-carboxamide+SX, N-propyl-1-({4-[5-(trifluoromethyl)-1,2,4-oxadiazol-3-yl]phenyl}methyl)-1H-pyrazole-4-carboxamide+SX, N-methoxy-1-({4-[5-(trifluoromethyl)-1,2,4-oxadiazol-3-yl]phenyl}methyl)-1H-pyrazole-4-carboxamide+SX, N-methoxy-N-methyl-1-({4-[5-(trifluoromethyl)-1,2,4-oxadiazol-3-yl]phenyl}methyl)-1H-pyrazole-4-carboxamide+SX, N,N-dimethyl-1-({4-[5-(trifluoromethyl)-1,2,4-oxadiazol-3-yl]phenyl}methyl)-1H-1,2,4-triazol-3-amine+SX, N-methyl-4-[5-(trifluoromethyl)-1,2,4-oxadiazol-3-yl]benzamide+SX, methyl 2-[2-chloro-4-(4-chlorophenoxy)phenyl]-2-hydroxy-3-(1H-1,2,4-triazol-1-yl)propanoate+SX, ethyl 2-[2-chloro-4-(4-chlorophenoxy)phenyl]-2-hydroxy-3-(1H-1,2,4-triazol-1-yl)propanoate+SX, methyl 2-[2-(trifluoromethyl)-4-(4-chlorophenoxy)phenyl]-2-hydroxy-3-(1H-1,2,4-triazol-1-yl)propanoate+SX, 1-(2,3-dimethylpyridin-5-yl)-4,4-difluoro-3,3-dimethyl-3,4-dihydroisoquinoline+SX, 1-[2-(difluoromethyl)-3-methylpyridin-5-yl]-4,4-difluoro-3,3-dimethyl-3,4-dihydroisoquinoline+SX, 2,2-difluoro-N-[6-({[1-(1-methyl-1H-tetrazol-5-yl)benzimidazol-2-yl]oxy}methyl)pyridin-2-yl]-2-phenoxyacetamide+SX, 1-[2-(1-chlorocyclopropyl)-3-(3-chloro-2-fluorophenyl)-2-hydroxypropyl]-1H-imidazole-5-carbonitrile+SX, ethyl 1-[(4-{[2-(trifluoromethyl)-1,3-dioxolan-2-yl]methoxy}phenyl)methyl]-1H-pyrazole-4-carboxylate+SX, ethyl 1-[(4-{[(1Z)-2-ethoxy-3,3,3-trifluoro-1-propen-1-yl]oxy}phenyl)methyl]-1H-pyrazole-4-carboxylate+SX, 6-chloro-3-(3-cyclopropyl-2-fluorophenoxy)-N-[2-(2,4-dimethylphenyl)-2,2-difluoroethyl]-5-methylpyridazine-4-carboxamide+SX, 6-chloro-3-(3-cyclopropyl-2-fluorophenoxy)-N-[2-(3,4-dimethylphenyl)-2,2-difluoroethyl]-5-methylpyridazine-4-carboxamide+SX, 6-chloro-N-[2-(2-chloro-4-methylphenyl)-2,2-difluoroethyl]-3-(3-cyclopropyl-2-fluorophenoxy)-5-methylpyridazine-4-carboxamide+SX, 2-[cyano (2,6-difluoropyridin-4-yl)amino]-N-(2,2-dimethylcyclobutyl)-5-methylthiazole-4-carboxamide+SX, 2-[cyano (2,6-difluoropyridin-4-yl)amino]-N-(spiro[3.4]octan-1-yl)-5-methylthiazole-4-carboxamide+SX, 2-[cyano (2,6-difluoropyridin-4-yl)amino]-N-hexyl-5-methylthiazole-4-carboxamide+SX, 2-[acetyl(2,6-difluoropyridin-4-yl)amino]-N-(2,2-dimethylcyclobutyl)-5-methylthiazole-4-carboxamide+SX, 2-[(2-methoxyacetyl) (2,6-difluoropyridin-4-yl)amino]-N-(2,2-dimethylcyclobutyl)-5-methylthiazole-4-carboxamide+SX, 2-[(2-methylpropanoyl) (2,6-difluoropyridin-4-yl)amino]-N-(2,2-dimethylcyclobutyl)-5-methylthiazole-4-carboxamide+SX, 5-[5-(difluoromethyl)-1,3,4-oxadiazol-2-yl]-N-[1-(2-fluorophenyl)ethyl]pyrimidin-2-amine+SX, 5-[5-(difluoromethyl)-1,3,4-oxadiazol-2-yl]-N-[1-(2,6-difluorophenyl)ethyl]pyrimidin-2-amine+SX, 5-[5-(difluoromethyl)-1,3,4-oxadiazol-2-yl]-N-[1-(3,5-difluorophenyl)ethyl]pyrimidin-2-amine+SX, 5-[5-(difluoromethyl)-1,3,4-oxadiazol-2-yl]-N-[1-(6-chloropyridin-3-yl)ethyl]pyrimidin-2-amine+SX, 5-[5-(difluoromethyl)-1,3,4-oxadiazol-2-yl]-N-[1-(2-fluorophenyl)cyclopropyl]pyrimidin-2-amine+SX, 5-[5-(difluoromethyl)-1,3,4-oxadiazol-2-yl]-N-[1-(2,6-difluorophenyl)cyclopropyl]pyrimidin-2-amine+SX, 5-[5-(difluoromethyl)-1,3,4-oxadiazol-2-yl]-N-[1-(2-fluoro-3-methoxyphenyl)cyclopropyl]pyrimidin-2-amine+SX, 5-[5-(difluoromethyl)-1,3,4-oxadiazol-2-yl]-2-{[1-(2,6-difluorophenyl)cyclopropyl]oxy}pyrimidine+SX, 3-[3-(3-cyclopropyl-2-fluorophenoxy)-6-methylpyridazin-4-yl]-5-[(2,4-dimethylphenyl)methyl]-5,6-dihydro-4H-1,2,4-oxadiazine+SX, (5S)-3-[3-(3-cyclopropyl-2-fluorophenoxy)-6-methylpyridazin-4-yl]-5-[(2,4-dimethylphenyl)methyl]-5,6-dihydro-4H-1,2,4-oxadiazine+SX, 3-[3-(3-chloro-2-fluorophenoxy)-6-methylpyridazin-4-yl]-5-[(4-bromo-2-methylphenyl)methyl]-5,6-dihydro-4H-1,2,4-oxadiazine+SX, 3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, (5S)-3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, (5R)-3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1 (2H)-yl}-3-methylbutan-2-yl)-2-methylpropanamide+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1 (2H)-yl}-3-methylbutan-2-yl)-2,2-dimethylpropanamide+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1 (2H)-yl}propan-2-yl)-2-methylpropanamide+SX, N-((2S)-1-{3-[2-(2-methoxyphenyl)-2-hydroxyethyl]-5-[(E)-1-(isopropoxyimino)ethyl]-2,6-dioxo-3,6-dihydropyrimidin-1 (2H)-yl}propan-2-yl)-2-methylpropanamide+SX, N-((2S)-1-{3-[2-(5-fluoro-2-methoxyphenyl)-2-(2-cyanoethoxy)ethyl]-5-[1-(isopropoxyimino)ethyl]-2,6-dioxo-3,6-dihydropyrimidin-1(2H)-yl}propan-2-yl)-2-methylpropanamide+SX, Agrobacterium radiobactor strain K1026+SX, Agrobacterium radiobactor strain K84+SX, Bacillus amyloliquefaciens strain PTA-4838 (Aveo (trademark) EZ Nematicide)+SX, Bacillus amyloliquefaciens strain AT332+SX, Bacillus amyloliquefaciens strain B3+SX, Bacillus amyloliquefaciens strain D747+SX, Bacillus amyloliquefaciens strain DB101+SX, Bacillus amyloliquefaciens strain DB102+SX, Bacillus amyloliquefaciens strain GB03+SX, Bacillus amyloliquefaciens strain FZB24+SX, Bacillus amyloliquefaciens strain FZB42+SX, Bacillus amyloliquefaciens strain IN937a+SX, Bacillus amyloliquefaciens strain MB1600+SX, Bacillus amyloliquefaciens strain QST713+SX, Bacillus amyloliquefaciens isolate strain B246+SX, Bacillus amyloliquefaciens strain F727+SX, Bacillus amyloliquefaciens subsp. plantarum strain D747+SX, Bacillus licheniformis strain HB-2+SX, Bacillus licheniformis strain SB3086+SX, Bacillus pumilus strain AQ717+SX, Bacillus pumilus strain BUF-33+SX, Bacillus pumilus strain GB34+SX, Bacillus pumilus strain QST2808+SX, Bacillus simplex strain CGF2856+SX, Bacillus subtilis strain AQ153+SX, Bacillus subtilis strain AQ743+SX, Bacillus subtilis strain BU1814+SX, Bacillus subtilis strain D747+SX, Bacillus subtilis strain DB101+SX, Bacillus subtilis strain FZB24+SX, Bacillus subtilis strain GB03+SX, Bacillus subtilis strain HAI0404+SX, Bacillus subtilis strain IAB/BS03+SX, Bacillus subtilis strain MBI600+SX, Bacillus subtilis strain QST30002/AQ30002+SX, Bacillus subtilis strain QST30004/AQ30004+SX, Bacillus subtilis strain QST713+SX, Bacillus subtilis strain QST714+SX, Bacillus subtilis var. Amyloliquefaciens strain FZB24+SX, Bacillus subtilis strain Y1336+SX, Burkholderia cepacia+SX, Burkholderia cepacia type Wisconsin strain J82+SX, Burkholderia cepacia type Wisconsin strain M54+SX, Candida oleophila strain O+SX, Candida saitoana+SX, Chaetomium cupreum+SX, Clonostachys rosea+SX, Coniothyrium minitans strain CGMCC8325+SX, Coniothyrium minitans strain CON/M/91-8+SX, cryptococcus albidus+SX, Erwinia carotovora subsp. carotovora strain CGE234M403+SX, Fusarium oxysporum strain Fo47+SX, Gliocladium catenulatum strain J1446+SX, Paenibacillus polymyxa strain AC-1+SX, Paenibacillus polymyxa strain BS-0105+SX, Pantoea agglomerans strain E325+SX, Phlebiopsis gigantea strain VRA1992+SX, Pseudomonas aureofaciens strain TX-1+SX, Pseudomonas chlororaphis strain 63-28+SX, Pseudomonas chlororaphis strain AFS009+SX, Pseudomonas chlororaphis strain MA342+SX, Pseudomonas fluorescens strain 1629RS+SX, Pseudomonas fluorescens strain A506+SX, Pseudomonas fluorescens strain CL145A+SX, Pseudomonas fluorescens strain G7090+SX, Pseudomonas sp. strain CAB-02+SX, Pseudomonas syringae strain 742RS+SX, Pseudomonas syringae strain MA-4+SX, Pseudozyma flocculosa strain PF-A22UL+SX, Pseudomonas rhodesiae strain HAI-0804+SX, Pythium oligandrum strain DV74+SX, Pythium oligandrum strain M1+SX, Streptomyces griseoviridis strain K61+SX, Streptomyces lydicus strain WYCD108US+SX, Streptomyces lydicus strain WYEC108+SX, Talaromyces flavus strain SAY-Y-94-01+SX, Talaromyces flavus strain V117b+SX, Trichoderma asperellum strain ICC012+SX, Trichoderma asperellum SKT-1+SX, Trichoderma asperellum strain T25+SX, Trichoderma asperellum strain T34+SX, Trichoderma asperellum strain TV1+SX, Trichoderma atroviride strain CNCM 1-1237+SX, Trichoderma atroviride strain LC52+SX, Trichoderma atroviride strain IMI 206040+SX, Trichoderma atroviride strain SC1+SX, Trichoderma atroviride strain SKT-1+SX, Trichoderma atroviride strain T11+SX, Trichoderma gamsii strain ICC080+SX, Trichoderma harzianum strain 21+SX, Trichoderma harzianum strain DB104+SX, Trichoderma harzianum strain DSM 14944+SX, Trichoderma harzianum strain ESALQ-1303+SX, Trichoderma harzianum strain ESALQ-1306+SX, Trichoderma harzianum strain IIHR-Th-2+SX, Trichoderma harzianum strain ITEM908+SX, Trichoderma harzianum strain kd+SX, Trichoderma harzianum strain MO1+SX, Trichoderma harzianum strain SF+SX, Trichoderma harzianum strain T22+SX, Trichoderma harzianum strain T39+SX, Trichoderma harzianum strain T78+SX, Trichoderma harzianum strain TH35+SX, Trichoderma polysporum strain IMI206039+SX, Trichoderma stromaticum+SX, Trichoderma virens strain G-41+SX, Trichoderma virens strain GL-21+SX, Trichoderma viride+SX, Variovorax paradoxus strain CGF4526+SX, and Harpin protein+SX.

Combination of the present component from the above group (c) and the compound of the present invention:
1-methylcyclopropene+SX, 1,3-diphenylurea+SX, 2,3,5-triiodobenzoic acid+SX, IAA ((1H-indol-3-yl)acetic acid)+SX, IBA (4-(1H-indol-3-yl) butyric acid)+SX, MCPA (2-(4-chloro-2-methylphenoxy)acetic acid)+SX, MCPB (4-(4-chloro-2-methylphenoxy) butyric acid)+SX, 4-CPA (4-chlorophenoxyacetic acid)+SX, 5-aminolevulinic acid hydrochloride+SX, 6-benzylaminopurine+SX, abscisic acid+SX, AVG (aminoethoxyvinylglycine)+SX, anisiflupurin+SX, ancymidol+SX, butralin+SX, calcium carbonate+SX, calcium chloride+SX, calcium formate+SX, calcium peroxide+SX, calcium polysulfide+SX, calcium sulfate+SX, chlormequat-chloride+SX, chlorpropham+SX, choline chloride+SX, cloprop+SX, cyanamide+SX, cyclanilide+SX, daminozide+SX, decan-1-ol+SX, dichlorprop+SX, dikegulac+SX, dimethipin+SX, diquat+SX, ethephon+SX, ethychlozate+SX, flumetralin+SX, flurprimidol+SX, forchlorfenuron+SX, formononetin+SX, Gibberellin A+SX, Gibberellin A3+SX, inabenfide+SX, Kinetin+SX, lipochitooligosaccharide SP104+SX, maleic hydrazide+SX, mefluidide+SX, mepiquat-chloride+SX, oxidized glutathione+SX, paclobutrazol+SX, pendimethalin+SX, prohexadione-calcium+SX, prohydrojasmon+SX, pyraflufen-ethyl+SX, sintofen+SX, sodium 1-naphthaleneacetate+SX, sodium cyanate+SX, thidiazuron+SX, triapenthenol+SX, tribufos+SX, trinexapac-ethyl+SX, uniconazole-P+SX, 2-(naphthalene-1-yl)acetamide (2-(naphthalen-1-yl)acetamide)+SX, [4-oxo-4-(2-phenylethyl)amino]butyrate+SX, 5-(trifluoromethyl)benzo[b]thiophene-2-methyl carboxylate+SX, 3-[(6-chloro-4-phenylquinazoline-2-yl)amino]propan-1-ol+SX, Claroideoglomus etunicatum+SX, Claroideoglomus claroideum+SX, Funneliformis mosseae+SX, Gigaspora margarita+SX, Gigaspora rosea+SX, Glomus aggregatum+SX, Glomus deserticola+SX, Glomus monosporum+SX, Paraglomus brasillianum+SX, Rhizophagus clarus+SX, Rhizophagus intraradices RTI-801+SX, Rhizophagus irregularis DAOM 197198+SX, Azorhizobium caulinodans+SX, Azospirillum amazonense+SX, Azospirillum brasilense XOH+SX, Azospirillum brasilense Ab-V5+SX, Azospirillum brasilense Ab-V6+SX, Azospirillum caulinodans+SX, Azospirillum halopraeferens+SX, Azospirillum irakense+SX, Azospirillum lipoferum+SX, Bradyrhizobium elkanii SEMIA 587+SX, Bradyrhizobium elkanii SEMIA 5019+SX, Bradyrhizobium japonicum TA-11+SX, Bradyrhizobium japonicum USDA 110+SX, Bradyrhizobium liaoningense+SX, Bradyrhizobium lupini+SX, Delftia acidovorans RAY209+SX, Mesorhizobium ciceri+SX, Mesorhizobium huakii+SX, Mesorhizobium loti+SX, Rhizobium etli+SX, Rhizobium galegae+SX, Rhizobium leguminosarum bv. Phaseoli+SX, Rhizobium leguminosarum bv. Trifolii+SX, Rhizobium leguminosarum bv. Viciae+SX, Rhizobium trifolii+SX, Rhizobium tropici+SX, Sinorhizobium fredii+SX, Sinorhizobium meliloti+SX, and Zucchini Yellow Mosaik Virus weak strain+SX.

Combination of the present component from the above group (d) and the compound of the present invention:
allidochlor+SX, benoxacor+SX, cloquintocet+SX, cloquintocet-mexyl+SX, cyometrinil+SX, cyprosulfamide+SX, dichlormid+SX, dicyclonone+SX, dimepiperate+SX, disulfoton+SX, dymron+SX, fenchlorazole+SX, fenchlorazole-ethyl+SX, fenclorim+SX, flurazole+SX, furilazole+SX, fluxofenim+SX, Hexim+SX, isoxadifen+SX, isoxadifen-ethyl+SX, Jiecaowan+SX, Jiecaoxi+SX, mecoprop+SX, mefenpyr+SX, mefenpyr-ethyl+SX, mefenpyr-diethyl+SX, mephenate+SX, metcamifen+SX, oxabetrinil+SX, 1,8-naphthalic anhydride+SX, 1,8-octamethylene diamine+SX, AD-67 (4-(dichloroacetyl)-1-oxa-4-azaspiro[4.5]decane)+SX, MCPA (2-(4-chloro-2-methylphenoxy)acetic acid)+SX, CL-304415 (4-carboxy-3,4-dihydro-2H-1-benzopyran-4-acetic acid)+SX, CSB (1-bromo-4-[(chloromethyl)sulfonyl] benzene)+SX, DKA-24 (2,2-dichloro-N-[2-oxo-2-(2-propenylamino)ethyl]-N-(2-propenyl)acetamide)+SX, MG191 (2-(dichloromethyl)-2-methyl-1,3-dioxolane)+SX, MG-838 (2-propenyl 1-oxa-4-azaspiro[4.5]decane-4-carbodithioate)+SX, PPG-1292 (2,2-dichloro-N-(1,3-dioxan-2-ylmethyl)-N-(2-propenyl)acetamide)+SX, R-28725 (3-(dichloroacetyl)-2,2-dimethyl-1,3-oxazolidine)+SX, R-29148 (3-(dichloroacetyl)-2,2,5-trimethyl-1,3-oxazolidine)+SX, and TI-35 (1-(dichloroacetyl) azepane)+SX.

Combination of the present component from the above group (e) and the compound of the present invention:
1-dodecyl-1H-imidazole+SX, N-(2-ethylhexyl)-8,9,10-trinorborn-5-ene-2,3-dicarboximide+SX, bucarpolate+SX, N,N-dibutyl-4-chlorobenzenesulfonamide+SX, dietholate+SX, diethylmaleate+SX, Jiajizengxiaolin+SX, octachlorodipropyl ether+SX, perbutin+SX, piperonyl butoxide+SX, piperonyl cyclonene+SX, piprotal+SX, propyl isome+SX, safroxan+SX, sesamex+SX, sesamolin+SX, sulfoxide+SX, Verbutin+SX, DMC (1,1-bis(4-chlorophenyl)ethanol)+SX, FDMC (1,1-bis(4-chlorophenyl)-2,2,2-trifluoroethanol)+SX, ETN (1,2-epoxy-1,2,3,4-tetrahydronaphthalene)+SX, ETP (1,1,1-trichloro-2,3-expoxypropane)+SX, PSCP (phenylsaligenin cyclic phosphate)+SX, TBPT (S,S,S-tributyl phosphorotrithioate)+SX, and TPP (triphenyl phosphate)+SX.

Combination of the present component from the above group (f) and the compound of the present invention:
2,3,6-TBA (2,3,6-trichlorobenzoic acid)+SX, 2,3,6-TBA-dimethylammonium+SX, 2,3,6-TBA-lithium+SX, 2,3,6-TBA-potassium+SX, 2,3,6-TBA-sodium+SX, 2,4-D+SX, 2,4-D choline salt+SX, 2,4-D-biproamine+SX, 2,4-D-doboxyl+SX, 2,4-D-2-ethylhexyl+SX, 2,4-D-3-butoxypropyl+SX, 2,4-D-ammonium+SX, 2,4-D-butotyl+SX, 2,4-D-butyl+SX, 2,4-D-diethylammonium+SX, 2,4-D-dimethylammonium+SX, 2,4-D-diolamine+SX, 2,4-D-dodecylammonium+SX, 2,4-D-ethyl+SX, 2,4-D-heptylammonium+SX, 2,4-D-isobutyl+SX, 2,4-D-isoctyl+SX, 2,4-D-isopropyl+SX, 2,4-D-isopropylammonium+SX, 2,4-D-lithium+SX, 2,4-D-mepty+SX, 2,4-D-methyl+SX, 2,4-D-octyl+SX, 2,4-D-pentyl+SX, 2,4-D-propyl+SX, 2,4-D-sodium+SX, 2,4-D-tefuryl+SX, 2,4-D-tetradecylammonium+SX, 2,4-D-triethylammonium+SX, 2,4-D-tris(2-hydroxypropyl) ammonium+SX, 2,4-D-trolamine+SX, 2,4-DB+SX, 2,4-DB choline salt+SX, 2,4-DB biproamine+SX, 2,4-DB-butyl+SX, 2,4-DB-dimethylammonium+SX, 2,4-DB-isoctyl+SX, 2,4-DB-potassium+SX, 2,4-DB-sodium+SX, acetochlor+SX, acifluorfen+SX, acifluorfen-sodium+SX, aclonifen+SX, ACN (2-amino-3-chloronaphthalene-1,4-dione)+SX, alachlor+SX, allidochlor+SX, alloxydim+SX, ametryn+SX, amicarbazone+SX, amidosulfuron+SX, aminocyclopyrachlor+SX, aminocyclopyrachlor-methyl+SX, aminocyclopyrachlor-potassium+SX, aminopyralid+SX, aminopyralid choline salt+SX, aminopyralid-potassium+SX, aminopyralid-tripromine+SX, amiprophos-methyl+SX, amitrole+SX, anilofos+SX, asulam+SX, atrazine+SX, azafenidin+SX, azimsulfuron+SX, beflubutamid+SX, benazolin-ethyl+SX, bencarbazone+SX, benfluralin+SX, benfuresate+SX, benquitrione+SX, bensulfuron+SX, bensulfuron-methyl+SX, bensulide+SX, bentazon+SX, benthiocarb+SX, benzfendizone+SX, benzobicyclon+SX, benzofenap+SX, benzthiazuron+SX, bialafosbialaphos+SX, bicyclopyrone+SX, bifenox+SX, bipyrazone+SX, bispyribac+SX, bispyribac-sodium+SX, bixlozone+SX, broclozone+SX, bromacil+SX, bromobutide+SX, bromofenoxim+SX, bromoxynil+SX, bromoxynil-octanoate+SX, butachlor+SX, butafenacil+SX, butamifos+SX, butralin+SX, butroxydim+SX, butylate+SX, cafenstrole+SX, carbetamide+SX, carfentrazone+SX, carfentrazone-ethyl+SX, chlomethoxyfen+SX, chloramben+SX, chloridazon+SX, chlorimuron+SX, chlorimuron-ethyl+SX, chlorobromuron+SX, chlorotoluron+SX, chloroxuron+SX, chlorpropham+SX, chlorsulfuron+SX, chlorthal-dimethyl+SX, chlorthiamid+SX, cinidon+SX, cinidon-ethyl+SX, cinmethylin+SX, cinosulfuron+SX, clethodim+SX, clodinafop+SX, clodinafop-propargyl+SX, clomazone+SX, clomeprop+SX, clopyralid+SX, clopyralid choline salt+SX, clopyralid-methyl+SX, clopyralid-olamine+SX, clopyralid-potassium+SX, clopyralid-tris(2-hydroxypropyl) ammonium+SX, cloransulam+SX, cloransulam-methyl+SX, cumyluron+SX, cyanazine+SX, cycloate+SX, cyclopyranil+SX, cyclopyrimorate+SX, cyclosulfamuron+SX, cycloxydim+SX, cyhalofop+SX, cyhalofop-butyl+SX, cypyrafluone+SX, daimuron+SX, dalapon+SX, dazomet+SX, desmedipham+SX, desmetryn+SX, di-allate+SX, dicamba+SX, dicamba choline salt+SX, dicamba-biproamine+SX, dicamba-trolamine+SX, dicamba-diglycolamine+SX, dicamba-dimethylammonium+SX, dicamba-diolamine+SX, dicamba-isopropylammonium+SX, dicamba-methyl+SX, dicamba-olamine+SX, dicamba-potassium+SX, dicamba-sodium+SX, dichlobenil+SX, dichlorprop+SX, dichlorprop choline salt+SX, dichlorprop-biproamine+SX, dichlorprop-etexyl+SX, dichlorprop-butotyl+SX, dichlorprop-dimethylammonium+SX, dichlorprop-ethylammonium+SX, dichlorprop-isoctyl+SX, dichlorprop-methyl+SX, dichlorprop-P+SX, dichlorprop-P choline salt+SX, dichlorprop-P-biproamine+SX, dichlorprop-P-etexyl+SX, dichlorprop-P-dimethylammonium+SX, dichlorprop-potassium+SX, dichlorprop-sodium+SX, diclofop+SX, diclofop-methyl+SX, diclosulam+SX, difenoxuron+SX, difenzoquat+SX, difenzoquat metilsulfate+SX, diflufenican+SX, diflufenzopyr+SX, diflufenzopyr-sodium+SX, dimefuron+SX, dimepiperate+SX, dimethachlor+SX, dimethametryn+SX, dimethenamid+SX, dimethenamid-P+SX, dimepiperate+SX, dinitramine+SX, dinoseb+SX, dinoterb+SX, dioxopyritrione+SX, diphenamid+SX, diquat+SX, diquat-dibromide+SX, DSMA (disodium methylarsonate)+SX, dithiopyr+SX, diuron+SX, DNOC (2-methyl-4,6-dinitrophenol)+SX, epyrifenacil+SX, esprocarb+SX, ethalfluralin+SX, ethametsulfuron+SX, ethametsulfuron-methyl+SX, ethidimuron+SX, ethofumesate+SX, ethoxyfen-ethyl+SX, ethoxysulfuron+SX, etobenzanid+SX, fenoxaprop+SX, fenoxaprop-ethyl+SX, fenoxaprop-P+SX, fenoxaprop-P-ethyl+SX, fenoxasulfone+SX, fenpyrazone+SX, fenquinotrione+SX, fentrazamide+SX, fenuron+SX, flamprop-M+SX, flazasulfuron+SX, florasulam+SX, florpyrauxifen+SX, florpyrauxifen-benzyl+SX, fluazifop+SX, fluazifop-butyl+SX, fluazifop-P+SX, fluazifop-P-butyl+SX, fluazolate+SX, flucarbazone+SX, flucarbazone-sodium+SX, flucetosulfuron+SX, fluchloraminopyr+SX, fluchloraminopyr-tefuryl+SX, flufenacet+SX, flufenoximacil+SX, flufenpyr+SX, flufenpyr-ethyl+SX, flumetsulam+SX, flumetsulam+SX, flumiclorac+SX, flumiclorac-pentyl+SX, flumioxazin+SX, fluometuron+SX, fluoroglycofen-ethyl+SX, flupoxam+SX, flupropanate+SX, flupyrsulfuron+SX, flupyrsulfuron-methyl-sodium+SX, flurenol+SX, fluridone+SX, flurochloridone+SX, fluroxypyr+SX, fluroxypyr-butometyl+SX, fluroxypyr-meptyl+SX, flurtamone+SX, flusulfinam+SX, fluthiacet+SX, fluthiacet-methyl+SX, fomesafen+SX, fomesafen-sodium+SX, foramsulfuron+SX, fosamine+SX, glufosinate+SX, glufosinate-ammonium+SX, glufosinate-P+SX, glufosinate-P-ammonium+SX, glufosinate-P-sodium+SX, glyphosate+SX, glyphosate choline salt+SX, glyphosate-isopropylammonium+SX, glyphosate-biproamine+SX, glyphosate-ammonium+SX, glyphosate-diammonium+SX, glyphosate-potassium+SX, glyphosate-sodium+SX, glyphosate-trimesium+SX, halauxifen+SX, halauxifen-benzyl+SX, halauxifen-methyl+SX, halosafen+SX, halosulfuron+SX, halosulfuron-methyl+SX, haloxyfop+SX, haloxyfop-etotyl+SX, haloxyfop-methyl+SX, haloxyfop-P+SX, haloxyfop-P-etotyl+SX, haloxyfop-P-methyl+SX, hexazinone+SX, icafolin+SX, icafolin-methyl+SX, imazamethabenz+SX, imazamethabenz-methyl+SX, imazamox+SX, imazamox-ammonium+SX, imazamox-sodium+SX, imazapic+SX, imazapic-ammonium+SX, imazapyr+SX, imazapyr-ammonium+SX, imazapyr-isopropylammonium+SX, imazaquin+SX, imazaquin-ammonium+SX, imazethapyr+SX, imazethapyr-ammonium+SX, imazosulfuron+SX, indanofan+SX, indaziflam+SX, indolauxipyr+SX, indolauxipyr-cyanomethyl+SX, iodosulfuron+SX, iodosulfuron-methyl-sodium+SX, iofensulfuron+SX, iofensulfuron-sodium+SX, ioxynil+SX, ioxynil-octanoate+SX, ipfencarbazone+SX, isoproturon+SX, isouron+SX, isoxaben+SX, isoxachlortole+SX, isoxaflutole+SX, lactofen+SX, lenacil+SX, linuron+SX, maleic hydrazide+SX, MCPA (2-(4-chloro-2-methylphenoxy)acetic acid)+SX, MCPA choline salt+SX, MCPA-biproamine+SX, MCPA etexyl+SX, MCPA butotyl+SX, MCPA-butyl+SX, MCPA-dimethylammonium+SX, MCPA-diolamine+SX, MCPA-ethyl+SX, MCPA-isobutyl+SX, MCPA-isoctyl+SX, MCPA-isopropyl+SX, MCPA-methyl+SX, MCPA-olamine+SX, MCPA-sodium+SX, MCPA-trolamine+SX, MCPB (4-(4-chloro-2-methylphenoxy)butanoic acid)+SX, MCPB choline salt+SX, MCPB-biproamine+SX, MCPB-ethyl+SX, MCPB-methyl+SX, MCPB-sodium+SX, mecoprop+SX, mecoprop choline salt+SX, mecoprop-biproamine+SX, mecoprop-2-ethylhexyl+SX, mecoprop-dimethylammonium+SX, mecoprop-diolamine+SX, mecoprop-ethadyl+SX, mecoprop-isoctyl+SX, mecoprop-methyl+SX, mecoprop-potassium+SX, mecoprop-sodium+SX, mecoprop-trolamine+SX, mecoprop-P+SX, mecoprop-P choline salt+SX, mecoprop-P-2-ethylhexyl+SX, mecoprop-P-dimethylammonium+SX, mecoprop-P-isobutyl+SX, mecoprop-P-potassium+SX, mefenacet+SX, mesosulfuron+SX, mesosulfuron-methyl+SX, mesotrione+SX, metam+SX, metamifop+SX, metamitron+SX, metazachlor+SX, metazosulfuron+SX, methabenzthiazuron+SX, methiozolin+SX, methyldymron+SX, metobromuron+SX, metolachlor+SX, metosulam+SX, metoxuron+SX, metribuzin+SX, metsulfuron+SX, metsulfuron-methyl+SX, molinate+SX, monolinuron+SX, naproanilide+SX, napropamide+SX, napropamide-M+SX, naptalam+SX, neburon+SX, nicosulfuron+SX, norflurazon+SX, oleic acid+SX, orbencarb+SX, orthosulfamuron+SX, oryzalin+SX, oxadiargyl+SX, oxadiazon+SX, oxasulfuron+SX, oxaziclomefone+SX, oxyfluorfen+SX, paraquat+SX, paraquat-dichloride+SX, pebulate+SX, pelargonic acid+SX, pendimethalin+SX, penoxsulam+SX, pentanochlor+SX, pentoxazone+SX, pethoxamid+SX, phenisopham+SX, phenmedipham+SX, picloram, picolinafen+SX, pinoxaden+SX, piperophos+SX, pretilachlor+SX, primisulfuron+SX, primisulfuron-methyl+SX, prodiamine+SX, profluazol+SX, profoxydim+SX, prometon+SX, prometryn+SX, propachlor+SX, propanil+SX, propaquizafop+SX, propazine+SX, propham+SX, propisochlor+SX, propoxycarbazone+SX, propoxycarbazone-sodium+SX, propyrisulfuron+SX, propyzamide+SX, prosulfocarb+SX, prosulfuron+SX, pyraclonil+SX, pyraquinate+SX, pyraflufen-ethyl+SX, pyrasulfotole+SX, pyrazolynate+SX, pyrazosulfuron+SX, pyrazosulfuron-ethyl+SX, pyrazoxyfen+SX, pyribenzoxim+SX, pyributicarb+SX, pyridafol+SX, pyridate+SX, pyriflubenzoxim+SX, pyriftalid+SX, pyriminobac+SX, pyriminobac-methyl+SX, pyrimisulfan+SX, pyrithiobac+SX, pyrithiobac-sodium+SX, pyroxasulfone+SX, pyroxsulam+SX, quinclorac+SX, quinmerac+SX, quizalofop+SX, quizalofop-ethyl+SX, quizalofop-tefuryl+SX, quizalofop-P+SX, quizalofop-P-ethyl+SX, quizalofop-P-tefuryl+SX, rimisoxafen+SX, rimsulfuron+SX, saflufenacil+SX, sethoxydim+SX, EPTC (S-ethyl N,N-dipropylcarbamothioate)+SX, siduron+SX, simazine+SX, simetryn+SX, S-metolachlor+SX, MSMA (sodium hydrogen methylarsonate)+SX, sulcotrione+SX, sulfentrazone+SX, sulfometuron+SX, sulfometuron-methyl+SX, sulfosulfuron+SX, swep+SX, TCA (2,2,2-trichloroacetic acid)+SX, TCA-ammonium+SX, TCA-calcium+SX, TCA-ethadyl+SX, TCA-magnesium+SX, TCA-sodium+SX, tebutam+SX, tebuthiuron+SX, tefuryltrione+SX, tembotrione+SX, tepraloxydim+SX, terbacil+SX, terbumeton+SX, terbuthylazine+SX, terbutryn+SX, tetflupyrolimet+SX, thaxtomin A+SX, thenylchlor+SX, thiazopyr+SX, thidiazimin+SX, thiencarbazone+SX, thiencarbazone-methyl+SX, thifensulfuron+SX, thifensulfuron-methyl+SX, tiafenacil+SX, tiocarbazil+SX, tolpyralate+SX, topramezone+SX, tralkoxydim+SX, triafamone+SX, tri-allate+SX, triasulfuron+SX, triaziflam+SX, tribenuron+SX, tribenuron-methyl+SX, triclopyr+SX, triclopyr-butotyl+SX, triclopyr-ethyl+SX, triclopyr-triethylammonium+SX, tridiphane+SX, trietazine+SX, trifloxysulfuron+SX, trifloxysulfuron-sodium+SX, trifludimoxazin+SX, trifluralin+SX, triflusulfuron+SX, triflusulfuron-methyl+SX, tripyrasulfone+SX, tritosulfuron+SX, vernolate+SX, 4-(4-fluorophenyl)-6-[(2-hydroxy-6-oxo-1-cyclohexene-1-yl)carbonyl]-2-methyl-1,2,4-triazine-3,5 (2H,4H)-dione+SX, 2-chloro-N-(1-methyl-1H-tetrazole-5-yl)-3-(methylthio)-4-(trifluoromethyl)benzamide+SX, 2-methyl-N-(5-methyl-1,3,4-oxadiazole-2-yl)-3-(methanesulfonyl)-4-(trifluoromethyl)benzamide+SX, and 1-{2-chloro-6-[(5-chloropyrimidin-2-yl)oxy]phenyl}-4,4,4-trifluorobutane-1-one+SX.

The combination of the present component of the above group (f) and the compound of the present invention is not limited, but for example, the following combination is more preferable. The number in parentheses is a preferred dose representing the treatment dosage (g) per hectare (ha) of each dose, but the present inventio is not limited thereto:
imazosulfuron+bromobutide+SX (90+900+20, 90+900+200), propyrisulfuron+bromobutide+SX (90+900+20, 90+900+200), methazosulfuron+bromobutide+SX (100+900+20, 100+900+200), triafamone+bromobutide+SX (50+900+20, 50+900+200), pyrimisulfan+bromobutide+SX (75+900+20, 75+900+200), bensulfuron-methyl+bromobutide+SX (51+900+20, 51+900+200, 75+900+20, 75+900+200), carfentrazone-ethyl+imazosulfuron+SX (90+90+20, 90+90+200), cumyluron+imazosulfuron+SX (1500+90+20, 1500+90+200), clomeprop+imazosulfuron+SX (350+90+20, 350+90+200), cyclopyranyl+imazosulfuron+SX (100+90+20, 100+90+200), cyclopylimylate+imazosulfuron+SX (300+90+20, 300+90+200), dimethatrin+imazosulfuron+SX (30+90+20, 30+90+200), daimuron+imazosulfuron+SX (450+90+20, 450+90+200), tefuryltrion+imazosulfuron+SX (300+90+20, 300+90+200), pyraclonil+imazosulfuron+SX (200+90+20, 200+90+200), pyrazoxyfen+imazosulfuron+SX (1000+90+20, 1000+90+200), pyrazolinate+imazosulfuron+SX (3000+90+20, 3000+90+200), fenquinotrion+imazosulfuron+SX (300+90+20, 300+90+200), florpyrauxyfenbenzyl+imazosulfuron+SX (50+90+20, 50+90+200), benzobicyclon+imazosulfuron+SX (200+90+20, 200+90+200), benzofenap+imazosulfuron+SX (800+90+20, 800+90+200), benfresate+imazosulfuron+SX (500+90+20, 500+90+200), mesotrione+imazosulfuron+SX (90+90+20, 90+90+200), lancotrione sodium salt+imazosulfuron+SX (210+90+20, 210+90+200), carfentrazone-ethyl+propyrisulfuron+SX (90+90+20, 90+90+200), cumylron+propylisulfuron+SX (1500+90+20, 1500+90+200), clomeprop+propyrisulfuron+SX (350+90+20, 350+90+200), cyclopyranyl+propyrisulfuron+SX (100+90+20, 100+90+200), cyclopylimilorate+propyrisulfuron+SX (300+90+20, 300+90+200), dimetatrin+propyrisulfuron+SX (30+90+20, 30+90+200), dymron+propyrisulfuron+SX (450+90+20, 450+90+200), tefuryltrion+propyrisulfuron+SX (300+90+20, 300+90+200), pyraclonil+propyrisulfuron+SX (200+90+20, 200+90+200), pyrazoxyfen+propyrisulfuron+SX (1000+90+20, 1000+90+200), pyrazolinate+propyrisulfuron+SX (3000+90+20, 3000+90+200), fenquinotrione+propyrisulfuron+SX (300+90+20, 300+90+200), florpirauxyfenbenzyl+propyrisulfuron+SX (50+90+20, 50+90+200), benzobicyclon+propyrisulfuron+SX (75200+90+20, 200+90+200), benzofenap+propyrisulfuron+SX (800+90+20, 800+90+200), benfresate+propyrisulfuron+SX (500+90+20, 500+90+200), mesotrione+propyrisulfuron+SX (90+90+20, 90+90+200), lancotrione sodium salt+propyrisulfuron+SX (210+90+20, 210+90+200), carfentrazone-ethyl+methazosulfuron+SX (90+100+20, 90+100+200), cumyluron+methazosulfuron+SX (1500+100+20, 1500+100+200), clomeprop+methazosulfuron+SX (350+100+20, 350+100+200), cyclopyranyl+methazosulfuron+SX (100+100+20, 100+100+200), cyclopylimylate+methazosulfuron+SX (300+100+20, 300+100+200), dimethatrin+methazosulfuron+SX (30+100+20, 30+100+200), daimuron+methazosulfuron+SX (450+100+20, 450+100+200), tefuryltrion+methazosulfuron+SX (300+100+20, 300+100+200), pyraclonil+methazosulfuron+SX (200+100+20, 200+100+200), pyrazoxyfen+methazosulfuron+SX (1000+100+20, 1000+100+200), pyrazolinate+methazosulfuron+SX (3000+100+20, 3000+100+200), fenquinotrione+methazosulfuron+SX (300+100+20, 300+100+200), florpirauxyfenbenzyl+methazosulfuron+SX (50+100+20, 50+100+200), benzobicyclon+methazosulfuron+SX (200+100+20, 200+100+200), benzofenap+methazosulfuron+SX (800+100+20, 800+100+200), benfresate+methazosulfuron+SX (500+100+20, 500+100+200), mesotrione+methazosulfuron+SX (90+100+20, 90+100+200), lancotrione sodium salt+methazosulfuron+SX (210+100+20, 210+100+200), carfentrazone-ethyl+triafamone+SX (90+50+20, 90+50+200), cumyluron+triafamone+SX (1500+50+20, 1500+50+200), clomeprop-triafamone+SX (350+50+20, 350+50+200), cyclopyranyl+triafamone+SX (100+50+20, 100+50+200), cyclopylimolate+triafamone+SX (300+50+20, 300+50+200), dimethatrin+triafamone+SX (30+50+20, 30+50+200), dymron+triafamone+SX (450+50+20, 450+50+200), tefuryltrione+triafamone+SX (300+50+20, 300+50+200), pyraclonil+triafamone+SX (200+50+20, 200+50+200), pyrazoxyfen+triafamone+SX (1000+50+20, 1000+50+200), pyrazolinate+triafamone+SX (3000+50+20, 3000+50+200), fenquinotrione+triafamone+SX (300+50+20, 300+50+200), florpiraxifenbenzyl+triafamone+SX (50+50+20, 50+50+200), benzobicyclon+triafamone+SX (200+50+20, 200+50+200), benzofenap+triafamone+SX (800+50+20, 800+50+200), benfrecate+triafamone+SX (500+50+20, 500+50+200), mesotrione+triafamone+SX (90+50+20, 90+50+200), lancotrion sodium salt+triafamone+SX (210+50+20, 210+50+200), carfentrazon-ethyl+pyrimisulfan+SX (90+75+20, 90+75+200), cumylon+pyrimisulfan+SX (1500+75+20, 1500+75+200), clomeprop+pyrimisulfan+SX (350+75+20, 350+75+200), cyclopyranyl+pyrimisulfan+SX (100+75+20, 100+75+200), cyclopylimolate+pyrimisulfan+SX (300+75+20, 300+75+200), dimethatrin+pyrimisulfan+SX (30+75+20, 30+75+200), daimuron+pyrimisulfan+SX (450+75+20, 450+75+200), tefuryltrione+pyrimisulfan+SX (300+75+20, 300+75+200), pyraclonil+pyrimisulfan+SX (200+75+20, 200+75+200), pyrazoxyfen+pyrimisulfan+SX (1000+75+20, 1000+75+200), pyrazolinate+pyrimisulfan+SX (3000+75+20, 3000+75+200), fenquinotrione+pyrimisulfan+SX (300+75+20, 300+75+200), florpirauxyfenbenzyl+pyrimisulfan+SX (50+75+20, 50+75+200), benzobicyclon+pyrimisulfan+SX (200+75+20, 200+75+200), benzofenap+pyrimisulfan+SX (800+75+20, 800+75+200), benfresate+pyrimisulfan+SX (500+75+20, 500+75+200), mesotrione+pyrimisulfan+SX (90+75+20, 90+75+200), lancotrione sodium salt+pyrimisulfan+SX (210+75+20, 210+75+200), carfentrazone-ethyl+bensulfuron-methyl+SX (90+51+20, 90+51+200, 90+75+20, 90+75+200), cumyluron+bensulfuron-methyl+SX (1500+51+20, 1500+51+200, 1500+75+20, 1500+75+200), clomeprop+bensulfuron-methyl+SX (350+51+20, 350+51+200, 350+75+20, 350+75+200), cyclopyranyl+bensulfuron-methyl+SX (100+51+20, 100+51+200, 100+75+20, 100+75+200), cyclopylimolate+bensulfuron-methyl+SX (300+51+20, 300+51+200, 300+75+20, 300+75+200), dimethatrin+bensulfuron-methyl+SX (30+51+20, 30+51+200, 30+75+20, 30+75+200), dymron+bensulfuron-methyl+SX (450+51+20, 450+51+200, 450+75+20, 450+75+200), tefuryltrione+bensulfuron-methyl+SX (300+51+20, 300+51+200, 300+75+20, 300+75+200), pyraclonil+bensulfuron-methyl+SX (200+51+20, 200+51+200, 200+75+20, 200+75+200), pyrazoxyfen+bensulfuron-methyl+SX (1000+51+20, 1000+51+200, 1000+75+20, 1000+75+200), pyrazolinate+bensulfuron-methyl+SX (3000+51+20, 3000+51+200, 3000+75+20, 3000+75+200), fenquinotrione+bensulfuron-methyl+SX (300+51+20, 300+51+200, 300+75+20, 300+75+200), florpirauxyfenbenzyl+bensulfuron-methyl+SX (50+51+20, 50+51+200, 50+75+20, 50+75+200), benzobicyclon+bensulfuron-methyl+SX (200+51+20, 200+51+200, 200+75+20, 200+75+200), benzofenap+bensulfuron-methyl+SX (800+51+20, 800+51+200, 800+75+20, 800+75+200), benfresate+bensulfuron-methyl+SX (500+51+20, 500+51+200, 500+75+20, 500+75+200), mesotrione+bensulfuron-methyl+SX (90+51+20, 90+51+200, 90+75+20, 90+75+200), lancotrione sodium salt+bensulfuron-methyl+SX (210+51+20, 210+51+200, 210+75+20, 210+75+200), carfentrazone-ethyl+bromobutide+SX (90+900+20, 90+900+200), cumylron+bromobutide+SX (1500+900+20, 1500+900+200), clomeprop+bromobutide+SX (350+900+20, 350+900+200), cyclopyranyl+bromobutide+SX (100+900+20, 100+900+200), cyclopylimolate+bromobutide+SX (300+900+20, 300+900+200), dimethatrin+bromobutide+SX (30+900+20, 30+900+200), dymron+bromobutide+SX (450+900+20, 450+900+200), tefuryltrione+bromobutide+SX (300+900+20, 300+900+200), pyraclonil+bromobutide+SX (200+900+20, 200+900+200), pyrazoxyfen+bromobutide+SX (1000+900+20, 1000+900+200), pyrazolinate+bromobutide+SX (3000+900+20, 3000+900+200), fenquinotrione+bromobutide+SX (300+900+20, 300+900+200), florpirauxifenbenzyl+bromobutide+SX (50+900+20, 50+900+200), benzobicyclon+bromobutide+SX (200+900+20, 200+900+200), benzofenap+bromobutide+SX (800+900+20, 800+900+200), benfresate+bromobutide+SX (500+900+20, 500+900+200), mesotrione+bromobutide+SX (90+900+20, 90+900+200), lancotrione sodium salt+bromobutide+SX (210+900+20, 210+900+200), carfentrazone-ethyl+imazosulfuron+bromobutide+SX (90+90+900+20, 90+90+900+200), cumylron+imazosulfuron+bromobutide+-SX (1500+90+900+20, 1500+90+900+200), clomeprop+imazosulfuron+bromobutide+SX (350+90+900+20, 350+90+900+200), cyclopyranyl+imazosulfuron+bromobutide SX (100+90+900+20, 100+90+900+200), cyclopylimolate+imazosulfuron+bromobutide+SX (300+90+900+20, 300+90+900+200), dimethatrin+imazosulfuron+bromobutide+SX (30+90+900+20, 30+90+900+200), daimuron+imazosulfuron+bromobutide+SX (450+90+900+20, 450+90+900+200), tefuryltrion+imazosulfuron+bromobutide+SX (300+90+900+20, 300+90+900+200), pyraclonil+imazosulfuron+bromobutide+SX (200+90+900+20, 200+90+900+200), pyrazoxyfen+imazosulfuron+bromobutide+SX (1000+90+900+20, 1000+90+900+200), pyrazolinate+imazosulfuron+bromobutide+SX (3000+90+900+20, 3000+90+900+200), fenquinotrione+imazosulfuron+bromobutide+SX (300+90+900+20, 300+90+900+200), florpirauxyfenbenzyl+imazosulfuron+bromobutide+SX (50+90+900+20, 50+90+900+200), benzobicyclon+imazosulfuron+bromobutide+SX (200+90+900+20, 200+90+900+200), benzofenap+imazosulfuron+bromobutide+SX (800+90+900+20, 800+90+900+200), benfresate+imazosulfuron+bromobutide+SX (500+90+900+20, 500+90+900+200), mesotrione+imazosulfuron+bromobutide+SX (90+90+900+20, 90+90+900+200), lancotrione sodium salt+imazosulfuron+bromobutide+SX (210+90+900+20, 210+90+900+200), carfentrazone-ethyl+propyrisulfuron+bromobutide+SX (90+90+900+20, 90+90+900+200), cumyluron+propylisulfuron+bromobutide+SX (1500+90+900+20, 1500+90+900+200), clomeprop+propylisulfuron+bromobutide+SX (350+90+900+20, 350+90+900+200), cyclopyranyl+propylisulfuron+bromobutide+SX (100+90+900+20, 100+90+900+200), cyclopylimylate+propylisulfuron+bromobutide+SX (300+90+900+20, 300+90+900+200), dimetatrin+propyrisulfuron+bromobutide+SX (30+90+900+20, 30+90+900+200), dymron+propyrisulfuron+bromobutide+SX (450+90+900+20, 450+90+900+200), tefuryltrion+propyrisulfuron+bromobutide+SX (300+90+900+20, 300+90+900+200), pyraclonil+propylisulfuron+bromobutide+SX (200+90+900+20, 200+90+900+200), pyrazoxyfen+propylisulfuron+bromobutide+SX (1000+90+900+20, 1000+90+900+200), pyrazolinate+propylisulfuron+bromobutide+SX (3000+90+900+20, 3000+90+900+200), fenquinotrione 1 propylisulfuron+bromobutide+SX (300+90+900+20, 300+90+900+200), florpirauxyfenbenzyl+propylisulfuron+bromobutide+SX (50+90+900+20, 50+90+900+200), benzobicyclon+propylisulfuron+bromobutide+SX (200+90+900+20, 200+90+900+200), benzofenap+propylisulfuron+bromobutide+SX (800+90+900+20, 800+90+900+200), benfresate+propyrisulfuron+bromobutide+SX (500+90+900+20, 500+90+900+200), mesotrione+propyrisulfuron+bromobutide+SX (90+90+900+20, 90+90+900+200), lancotrione sodium salt+propyrisulfuron+bromobutide+SX (210+90+900+20, 210+90+900+200), carfentrazone-ethyl+methazosulfuron 1 bromobutide 1 SX (90+100+900+20, 90+100+900+200), cumylron+methazosulfuron+bromobutide+SX (1500+100+900+20, 1500+100+900+200), clomeprop+methazosulfuron+bromobutide+SX (350+100+900+20, 350+100+900+200), cyclopyranyl+methazosulfuron+bromobutide+SX (100+100+900+20, 100+100+900+200), cyclopylimolate+methazosulfuron+bromobutide+SX (300+100+900+20, 300+100+900+200), dimetatrin+methazosulfuron+bromobutide+SX (30+100+900+20, 30+100+900+200), daimuron+methazosulfuron+bromobutide+SX (450+100+900+20, 450+100+900+200), tefuryltrion+methazosulfuron+bromobutide+SX (300+100+900+20, 300+100+900+200), pyraclonil+methazosulfuron+bromobutide+SX (200+100+900+20, 200+100+900+200), pyrazoxyfen 1 methazosulfuron+bromobutide+SX (1000+100+900+20, 1000+100+900+200), pyrazolinate+methazosulfuron+bromobutide+SX (3000+100+900+20, 3000+100+900+200), fenquinotrione+methazosulfuron-bromobutide+SX (300+100+900+20, 300+100+900+200), florpirauxyfenbenzyl+methazosulfuron+bromobutide+SX (50+100+900+20, 50+100+900+200), benzobicyclon+methazosulfuron+bromobutide+SX (200+100+900+20, 200+100+900+200), benzofenap+methazosulfuron+bromobutide+SX (800+100+900+20, 800+100+900+200), benfresate+methazosulfuron+bromobutide+SX (500+100+900+20, 500+100+900+200), mesotrione+methazosulfuron+bromobutide+SX (90+100+900+20, 90+100+900+200), lancotrione sodium salt+methazosulfuron+bromobutide+SX (210+100+900+20, 210+100+900+200), carfentrazone-ethyl+triafamone+bromobutide+SX (90+50+900+20, 90+50+900+200), cumylron+triafamone+bromobutide+SX (1500+50+900+20, 1500+50+900+200), clomeprop+triafamone+bromobutide+SX (350+50+900+20, 350+50+900+200), cyclopyranyl+triafamone+bromobutide+SX (100+50+900+20, 100+50+900+200), cyclopylimolate+triafamone+bromobutide+SX (300+50+900+20, 300+50+900+200), dimethatrin+triafamone+bromobutide+SX (30+50+900+20, 30+50+900+200), dymron+triafamone+bromobutide+SX (450+50+900+20, 450+50+900+200), tefuryltrione+triafamone+bromobutide+SX (300+50+900+20, 300+50+900+200), pyraclonil+triafamone+bromobutide+SX (200+50+900+20, 200+50+900+200), pyrazoxyfen+triafamone+bromobutide+SX (1000+50+900+20, 1000+50+900+200), pyrazolinate+triafamone+bromobutide+SX (3000+50+900+20, 3000+50+900+200), fenquinotrione+triafamone+bromobutide+SX (300+50+900+20, 300+50+900+200), florpirauxyfenbenzyl+triafamone+bromobutide+SX (50+50+900+20, 50+50+900+200), benzobicyclon+triafamone+bromobutide+SX (200+50+900+20, 200+50+900+200), benzofenap+triafamone+bromobutide+SX (800+50+900+20, 800+50+900+200), benfresate+triafamone+bromobutide+SX (500+50+900+20, 500+50+900+200), mesotrione+triafamone+bromobutide+SX (90+50+900+20, 90+50+900+200), lancotrion sodium salt+triafamone+bromobutide+SX (210+50+900+20, 210+50+900+200), carfentrazone-ethyl+pyrimisulfan+bromobutide+SX (90+75+900+20, 90+75+900+200), cumylron+pyrimisulfan+bromobutide+SX (1500+75+900+20, 1500+75+900+200), clomeprop+pyrimisulfan+bromobutide+SX (350+75+900+20, 350+75+900+200), cyclopyranyl+pyrimisulfan+bromobutide+SX (100+75+900+20, 100+75+900+200), cyclopylimolate+pyrimisulfan+bromobutide+SX (300+75+900+20, 300+75+900+200), dimethatrin+pyrimisulfan+bromobutide+SX (30+75+900+20, 30+75+900+200), daimuron+pyrimisulfan bromobutide+SX (450+75+900+20, 450+75+900+200), tefuryltrione+pyrimisulfan+bromobutide+SX (300+75+900+20, 300+75+900+200), pyraclonil+pyrimisulfan+bromobutide+SX (200+75+900+20, 200+75+900+200), pyrazoxyfen+pyrimisulfan+bromobutide+SX (1000+75+900+20, 1000+75+900+200), pyrazolinate+pyrimisulfan+bromobutide+SX (3000+75+900+20, 3000+75+900+200), fenquinotrione+pyrimisulfan+bromobutide+SX (300+75+900+20, 300+75+900+200), florpirauxyfenbenzyl+pyrimisulfan+bromobutide+SX (50+75+900+20, 50+75+900+200), benzobicyclon+pyrimisulfan+bromobutide+SX (200+75+900+20, 200+75+900+200), benzofenap+pyrimisulfan+bromobutide SX (800+75+900+20, 800+75+900+200), benfresate+pyrimisulfan+bromobutide+SX (500+75+900+20, 500+75+900+200), mesotrione+pyrimisulfan+bromobutide+SX (90+75+900+20, 90+75+900+200), lancotrione sodium salt+pyrimisulfan+bromobutide+SX (210+75+900+20, 210+75+900+200), carfentrazone-ethyl+bensulfuron-methyl+bromobutide+SX (90+51+900+20, 90+51+900+200, 90+75+900+20, 200+75+900+200), cumylron+bensulfuron-methyl+bromobutide+SX (1500+51+900+20, 1500+51+900+200, 1500+75+900+20, 200+75+900+200), clomeprop+bensulfuron-methyl+bromobutide+SX (350+51+900+20, 350+51+900+200, 350+75+900+20, 200+75+900+200), cyclopyranyl+bensulfuron-methyl+bromobutide+SX (100+51+900+20, 100+51+900+200, 100+75+900+20, 200+75+900+200), cyclopylimolate+bensulfuron-methyl+bromobutide+SX (300+51+900+20, 300+51+900+200, 300+75+900+20, 200+75+900+200), dimetatrin+bensulfuron-methyl+bromobutide+SX (30+51+900+20, 30+51+900+200, 30+75+900+20, 200+75+900+200), dymron+bensulfuron-methyl+bromobutide+SX (450+51+900+20, 450+51+900+200, 450+75+900+20, 200+75+900+200), tefuryltrione+bensulfuron-methyl+bromobutide+SX (300+51+900+20, 300+51+900+200, 300+75+900+20, 200+75+900+200), pyraclonil+bensulfuron-methyl+bromobutide+SX (200+51+900+20, 200+51+900+200, 200+75+900+20, 200+75+900+200), pyrazoxyfen+bensulfuron-methyl+bromobutide+SX (1000+51+900+20, 1000+51+900+200, 1000+75+900+20, 200+75+900+200), pyrazolinate+bensulfuron-methyl+bromobutide+SX (3000+51+900+20, 3000+51+900+200, 3000+75+900+20, 200+75+900+200), fenquinotrione+bensulfuron-methyl+bromobutide+SX (300+51+900+20, 300+51+900+200, 300+75+900+20, 200+75+900+200), florpirauxyfenbenzyl+bensulfuron-methyl+bromobutide+SX (50+51+900+20, 50+51+900+200, 50+75+900+20, 200+75+900+200), benzobicyclon+bensulfuron-methyl+bromobutide+SX (200+51+900+20, 200+51+900+200, 200+75+900+20, 200+75+900+200), benzofenap+bensulfuron-methyl+bromobutide+SX (800+51+900+20, 800+51+900+200, 800+75+900+20, 200+75+900+200), benfresate+bensulfuron-methyl+bromobutide+SX (500+51+900+20, 500+51+900+200, 500+75+900+20, 200+75+900+200), mesotrione+bensulfuron-methyl+bromobutide+SX (90+51+900+20, 90+51+900+200, 90+75+900+20, 200+75+900+200), lancotrione sodium salt+bensulfuron-methyl+bromobutide+SX (210+51+900+20, 210+51+900+200, 210+75+900+20, 200+75+900+200), carfentrazone-ethyl 1 imazosulfuron+dimesulfazet+SX (90+90+150+20, 90+90+150+200), cumyluron+imazosulfuron+dimesulfazet+SX (1500+90+150+20, 1500+90+150+200), clomeprop+imazosulfuron+dimesulfazet+SX (350+90+150+20, 350+90+150+200), cyclopyranyl+imazosulfuron+dimesulfazet+SX (100+90+150+20, 100+90+150+200), cyclopylimylate+imazosulfuron+dimesulfazet+SX (300+90+150+20, 300+90+150+200), dimethatrin+imazosulfuron+dimesulfazet+SX (30+90+150+20, 30+90+150+200), daimuron+imazosulfuron+dimesulfazet+SX (450+90+150+20, 450+90+150+200), tefuryltrion+imazosulfuron+dimesulfazet+SX (300+90+150+20, 300+90+150+200), pyraclonil+imazosulfuron+dimesulfazet+SX (200+90+150+20, 200+90+150+200), pyrazoxyfen+imazosulfuron+dimesulfazet+SX (1000+90+150+20, 1000+90+150+200), pyrazolinate+imazosulfuron+dimesulfazet+SX (3000+90+150+20, 3000+90+150+200), fenquinotrione+imazosulfuron+dimesulfazet+SX (300+90+150+20, 300+90+150+200), florpirauxifenbenzyl+imazosulfuron+dimesulfazet+SX (50+90+150+20, 50+90+150+200), benzobicyclon+imazosulfuron+dimesulfazet+SX (200+90+150+20, 200+90+150+200), benzofenap+imazosulfuron+dimesulfazet+SX (800+90+150+20, 800+90+150+200), benfresate+imazosulfuron+dimesulfazet+SX (500+90+150+20, 500+90+150+200), mesotrione+imazosulfuron+dimesulfazet+SX (90+90+150+20, 90+90+150+200), lancotrione sodium salt+imazosulfuron+dimesulfazet+SX (210+90+150+20, 210+90+150+200), carfentrazone-ethyl+propyrisulfuron+dimesulfazet+SX (90+90+150+20, 90+90+150+200), cumyluron+propylisulfuron+dimesulaset+SX (1500+90+150+20, 1500+90+150+200), clomeprop+propyrisulfuron+dimesulaset+SX (350+90+150+20, 350+90+150+200), cyclopyranyl+propyrisulfuron+dimesulaset+SX (100+90+150+20, 100+90+150+200), cyclopylimylate+propyrisulfuron+dimesulaset+SX (300+90+150+20, 300+90+150+200), dimetatrin+propyrisulfuron+dimesulfazet+SX (30+90+150+20, 30+90+150+200), dimuron+propyrisulfuron+dimesulfazet+SX (450+90+150+20, 450+90+150+200), tefuryltrion+propyrisulfuron+dimesulfazet+SX (300+90+150+20, 300+90+150+200), pyraclonil+propyrisulfuron+dimesulfazet+SX (200+90+150+20, 200+90+150+200), pyrazoxyfen+propyrisulfuron+dimesulfazet+SX (1000+90+150+20, 1000+90+150+200), pyrazolinate+propyrisulfuron+dimesulfazet+SX (3000+90+150+20, 3000+90+150+200), fenquinotrione+propyrisulfuron+dimesulfazet+SX (300+90+150+20, 300+90+150+200), florpirauxyfenbenzyl propyrisulfuron+dimesulfazet+SX (50+90+150+20, 50+90+150+200), benzobicyclon+propyrisulfuron+dimesulfazet+SX (200+90+150+20, 200+90+150+200), benzofenap+propyrisulfuron+dimesulfazet+SX (800+90+150+20, 800+90+150+200), benfrecate+propyrisulfuron+dimesulfazet+SX (500+90+150+20, 500+90+150+200), mesotrione+propyrisulfuron+dimesulfazet+SX (90+90+150+20, 90+90+150+200), lancotrione sodium salt+propyrisulfuron+dimesulfazet+SX (210+90+150+20, 210+90+150+200), carfentrazone-ethyl metazosulfuron+dimesulfazet+SX (90+100+150+20, 90+100+150+200), cumyluron+metazosulfuron+dimesulfazet+SX (1500+100+150+20, 1500+100+150+200), clomeprop+metazosulfuron+dimesulfazet+SX (350+100+150+20, 350+100+150+200), cyclopyranyl+methazosulfuron+dimesulfazet+SX (100+100+150+20, 100+100+150+200), cyclopylimylate+metazosulfuron+dimesulfazet+SX (300+100+150+20, 300+100+150+200), dimethatrin+metazosulfuron+dimesulfazet+SX (30+100+150+20, 30+100+150+200), dymron+methazosulfuron+dimesulfazet+SX (450+100+150+20, 450+100+150+200), tefuryltrione+methazosulfuron-+-dimesulfazet-+-SX (300+100+150+20, 300+100+150+200), pyraclonil+methazosulfuron+dimesulfazet+SX (200+100+150+20, 200+100+150+200), pyrazoxyfen+methazosulfuron+dimesulfazet+SX (1000+100+150+20, 1000+100+150+200), pyrazolinate+metazosulfuron+dimesulfazet+SX (3000+100+150+20, 3000+100+150+200), fenquinotrione+metazosulfuron+dimesulfazet+SX (300+100+150+20, 300+100+150+200), florpirauxyfenbenzyl metazosulfuron+dimesulfazet+SX (50+100+150+20, 50+100+150+200), benzobicyclon+metazosulfuron+dimesulfazet+SX (200+100+150+20, 200+100+150+200), benzofenap+methazosulfuron+dimesulfazet+SX (800+100+150+20, 800+100+150+200), benfresate+methazosulfuron+dimesulfazet+SX (500+100+150+20, 500+100+150+200), mesotrione+methazosulfuron+dimesulfazet+SX (90+100+150+20, 90+100+150+200), lancotrione sodium salt+methazosulfuron+dimesulfazet SX (210+100+150+20, 210+100+150+200), carfentrazone-ethyl+triafamone+dimesulaset+SX (90+50+150+20, 90+50+150+200), cumyluron+triafamone+dimesulaset+SX (1500+50+150+20, 1500+50+150+200), clomeprop+triafamone+dimesulaset+SX (350+50+150+20, 350+50+150+200), cyclopyranyl+triafamone+dimesulfazet+SX (100+50+150+20, 100+50+150+200), cyclopylimolate+triafamone+dimesulfazet+SX (300+50+150+20, 300+50+150+200), dimethatrin+triafamone+dimesulfazet+SX (30+50+150+20, 30+50+150+200), dymron+triafamone+dimesulfazet+SX (450+50+150+20, 450+50+150+200), tefuryltrione+triafamone+dimesulfazet+SX (300+50+150+20, 300+50+150+200), pyraclonil+triafamone+dimesulfazet+SX (200+50+150+20, 200+50+150+200), pyrazoxyfen+triafamone+dimesulaset+SX (1000+50+150+20, 1000+50+150+200), pyrazolinate+triafamone+dimesulaset+SX (3000+50+150+20, 3000+50+150+200), fenquinotrione+triafamone+dimesulaset+SX (300+50+150+20, 300+50+150+200), florpirauxyfenbenzyl+triafamone+dimesulaset+SX (50+50+150+20, 50+50+150+200), benzobicyclon+triafamone+dimesulaset+SX (200+50+150+20, 200+50+150+200), benzofenap+triafamone+dimesulaset+SX (800+50+150+20, 800+50+150+200), benphrecate+triafamone+dimesulaset+SX (500+50+150+20, 500+50+150+200), mesotrione+triafamone+dimesulaset+SX (90+50+150+20, 90+50+150+200), lancotrione sodium salt+triafamone+dimesulaset+SX (210+50+150+20, 210+50+150+200), carfentrazone-ethyl+pyrimisulfan+dimesulaset+SX (90+75+150+20, 90+75+150+200), cumylron+pyrimisulfan+dimesulaset+SX (1500+75+150+20, 1500+75+150+200), clomeprop+pyrimisulfan+dimesulaset+SX (350+75+150+20, 350+75+150+200), cyclopyranyl+pyrimisulfan+dimesulaset+SX (100+75+150+20, 100+75+150+200), cyclopylimylate+pyrimisulfan+dimesulfazet+SX (300+75+150+20, 300+75+150+200), dimetatrin+pyrimisulfan+dimesulfazet+SX (30+75+150+20, 30+75+150+200), dimuron+pyrimisulfan+dimesulfazet+SX (450+75+150+20, 450+75+150+200), tefuryltrione+pyrimisulfan+dimesulaset+SX (300+75+150+20, 300+75+150+200), pyraclonil+pyrimisulfan+dimesulaset+SX (200+75+150+20, 200+75+150+200), pyrazoxyfen+pyrimisulfan+dimesulaset+SX (1000+75+150+20, 1000+75+150+200), pyrazolinate+pyrimisulfan+dimesulaset+SX (3000+75+150+20, 3000+75+150+200), fenquinotrione+pyrimisulfan+dimesulfazet+SX (300+75+150+20, 300+75+150+200), florpirauxyfenbenzyl+pyrimisulfan+dimesulfazet+SX (50+75+150+20, 50+75+150+200), benzobicyclon+pyrimisulfan+dimesulfazet+SX (200+75+150+20, 200+75+150+200), benzofenap+pyrimisulfan+dimesulfazet+SX (800+75+150+20, 800+75+150+200), benfresate+pyrimisulfan+dimesulaset+SX (500+75+150+20, 500+75+150+200), mesotrione+pyrimisulfan+dimesulaset+SX (90+75+150+20, 90+75+150+200), lancotrione sodium salt+pyrimisulfan+dimesulaset+SX (210+75+150+20, 210+75+150+200), carfentrazone-ethyl+benzulfuron-methyl+dimesulaset+SX (90+51+150+20, 90+51+150+200, 90+75+150+20, 200+75+150+200), cumyluron+bensulfuron-methyl+dimesulaset+SX (1500+51+150+20, 1500+51+150+200, 1500+75+150+20, 200+75+150+200), clomeprop+bensulfuron-methyl+dimesulaset+SX (350+51+150+20, 350+51+150+200, 350+75+150+20, 200+75+150+200), cyclopyranyl+bensulfuron-methyl+dimesulaset+SX (100+51+150+20, 100+51+150+200, 100+75+150+20, 200+75+150+200), cyclopyrrimolate+benzulfuron-methyl+dimesulfazet 1 SX (300+51+150+20, 300+51+150+200, 300+75+150+20, 200+75+150+200), dimetatrin+benzulfuron-methyl+dimesulfazet+SX (30+51+150+20, 30+51+150+200, 30+75+150+20, 200+75+150+200), daimuron+benzulfuron-methyl+dimesulfazet+SX (450+51+150+20, 450+51+150+200, 450+75+150+20, 200+75+150+200), tefuryltrione+benzulfuron-methyl+dimesulaset+SX (300+51+150+20, 300+51+150+200, 300+75+150+20, 200+75+150+200), pyraclonil+benzulfuron-methyl+dimesulaset+SX (200+51+150+20, 200+51+150+200, 200+75+150+20, 200+75+150+200), pyrazoxyfen+benzulfuron-methyl+dimesulaset+SX (1000+51+150+20, 1000+51+150+200, 1000+75+150+20, 200+75+150+200), pyrazolinate+bensulfuron-methyl+dimesulfazet+SX (3000+51+150+20, 3000+51+150+200, 3000+75+150+20, 200+75+150+200), fenquinotrione+bensulfuron-methyl+dimesulfazet+SX (300+51+150+20, 300+51+150+200, 300+75+150+20, 200+75+150+200), florpirauxyfenbenzyl+bensulfuron-methyl+dimesulfazet+SX (50+51+150+20, 50+51+150+200, 50+75+150+20, 200+75+150+200), benzobicyclon+benzulfuron-methyl+dimesulaset+SX (200+51+150+20, 200+51+150+200, 200+75+150+20, 200+75+150+200), benzofenap+benzulfuron-methyl+dimesulaset+SX (800+51+150+20, 800+51+150+200, 800+75+150+20, 200+75+150+200), benfresate+benzulfuron-methyl+dimesulaset+SX (500+51+150+20, 500+51+150+200, 500+75+150+20, 200+75+150+200), mesotrione+benzulfuron-methyl+dimesulaset+SX (90+51+150+20, 90+51+150+200, 90+75+150+20, 200+75+150+200), iancotrione sodium salt+bensulfuron-methyl+dimesulfazet-+-SX (210+51+150+20, 210+51+150+200, 210+75+150+20, 200+75+150+200), imazosulfuron+cyclopylimolate+tefuryltrion+SX (90+300+300+20, 90+300+300+200), propyrisulfuron+cyclopylimolate tefuryltrion 1 SX (90+300+300+20, 90+300+300+200), methazosulfuron+cyclopylimolate+tefuryltrion+SX (100+300+300+20, 100+300+300+200), triafamone+cyclopylimolate+tefuryltrione+SX (50+300+300+20, 50+300+300+200), pyrimisulfan+cyclopylimolate+tefuryltrione+SX (75+300+300+20, 75+300+300+200), bensulfuron-methyl cyclopylimolate+tefuryltrione+SX (51+300+300+20, 51+300+300+200, 75+300+300+20, 200+300+300+200), imazosulfuron+cyclopylimolate+mesotrione+SX (90+300+90+20, 90+300+90+200), propyrisulfuron+cyclopylimolate+mesotrione+SX (90+300+90+20, 90+300+90+200), methazosulfuron+cyclopylimolate+mesotrione+SX (100+300+90+20, 100+300+90+200), triafamone+cyclopylimolate+mesotrione+SX (50+300+90+20, 50+300+90+200), pyrimisulfan+cyclopylimolate+mesotrione+SX (75+300+90+20, 75+300+90+200), benzsulfuron-methyl+cyclopylimolate+mesotrione+SX (51+300+90+20, 51+300+90+200, 75+300+90+20, 200+300+90+200), imazosulfuron+cyclopylimolate+benzobicyclon+SX (90+300+200+20, 90+300+200+200), propylisulfuron+cyclopylimolate+benzobicyclon+SX (90+300+200+20, 90+300+200+200), methazosulfuron+cyclopylimolate+benzobicyclon+SX (100+300+200+20, 100+300+200+200), triafamone+cyclopylimolate+benzobicyclon+SX (50+300+200+20, 50+300+200+200), pyrimisulfan+cyclopylimolate+benzobicyclon+SX (75+300+200+20, 75+300+200+200), bensulfuron-methyl+cyclopylimolate 1 benzobicyclon+SX (51+300+200+20, 51+300+200+200, 75+300+200+20, 200+300+200+200), imazosulfuron+cyclopylimolate+fenquinotrione+SX (90+300+300+20, 90+300+300+200), propyrisulfuron+cyclopylimolate+fenquinotrione+SX (90+300+300+20, 90+300+300+200), methazosulfuron+cyclopylimolate+fenquinotrione+SX (100+300+300+20, 100+300+300+200), triafamone+cyclopylimolate+fenquinotrione+SX (50+300+300+20, 50+300+300+200), pyrimisulfan+cyclopylimolate+fenquinotrione+SX (75+300+300+20, 75+300+300+200), bensulfuron-methyl+cyclopylimolate+fenquinotrione SX (51+300+300+20, 51+300+300+200, 75+300+300+20, 200+300+300+200), imazosulfuron+cyclopylimolate+pyrazolinate+SX (90+300+3000+20, 90+300+3000+200), propyrisulfuron+cyclopylimolate+pyrazolinate+SX (90+300+3000+20, 90+300+3000+200), methazosulfuron-+cyclopylimolate+pyrazolinate+SX (100+300+3000+20, 100+300+3000+200), triafamone+cyclopylimolate+pyrazolinate+SX (50+300+3000+20, 50+300+3000+200), pyrimisulfan+cyclopylimolate+pyrazolinate+SX (75+300+3000+20, 75+300+3000+200), bensulfuron-methyl+cyclopylimolate+pyrazolinate+SX (51+300+3000+20, 51+300+3000+200, 75+300+3000+20, 200+300+3000+200), imazosulfuron+cyclopylimolate+lancotrione sodium salt+SX (90+300+210+20, 90+300+210+200), propyrisulfuron+cyclopylimolate+lancotrione sodium salt+SX (90+300+210+20, 90+300+210+200), methazosulfuron+cyclopylimolate+lancotrione sodium salt+SX (100+300+210+20, 100+300+210+200), triafamone+cyclopylimolate+lancotrione sodium salt+SX (50+300+210+20, 50+300+210+200), pyrimisulfan+cyclopylimolate+lancotrione sodium salt+SX (75+300+210+20, 75+300+210+200), and bensulfuron-methyl+cyclopylimolate+lancotrione sodium salt+SX (51+300+210+20, 51+300+210+200, 75+300+210+20, 200+300+210+200)

Specific examples of a control target of the compound of the present invention include, but are not limited to, the following.

Urticaceae: Urtica urens

Polygonaceae: Polygonum convolvulus, Polygonum lapathifolium, Polygonum pensylvanicum, Polygonum persicaria, Polygonum longisetum, Polygonum aviculare, Polygonum arenastrum, Polygonum cuspidatum, Rumex japonicus, Rumex crispus, Rumex obtusifolius, and Rumex acetosa

Portulacaceae: Portulaca oleracea

Caryophyllaceae: Stellaria media, Stellaria aquatica, Cerastium holosteoides, Cerastium glomeratum, Spergula arvensis, and Silene gallica

Molluginaceae: Mollugo verticillata

Chenopodiaceae: Chenopodium album, Chenopodium ambrosioides, Kochia scoparia, Salsola kali, and Atriplex spp.

Amaranthaceae: Amaranthus retroflexus, Amaranthus viridis, Amaranthus lividus, Amaranthus spinosus, Amaranthus hybridus, Amaranthus palmeri, Amaranthus patulus, Waterhemp (Amaranthus tuberculatus=Amaranthus rudis=Amaranthus tamariscinus), Amaranthus blitoides, Amaranthus deflexus, Amaranthus quitensis, Alternanthera philoxeroides, Alternanthera sessilis, and Alternanthera tenella

Papaveraceae: Papaver rhoeas, Papaver dubium, and Argemone mexicana

Brassicaceae: Raphanus raphanistrum, Raphanus sativus, Sinapis arvensis, Capsella bursa-pastoris, Brassica juncea, Brassica napus, Descurainia pinnata, Rorippa islandica, Rorippa sylvestris, Thlaspi arvense, Myagrum rugosum, Lepidium virginicum, and Coronopus didymus

Capparaceae: Cleome affinis

Fabaceae: Aeschynomene indica, Aeschynomene rudis, Sesbania exaltata, Cassia obtusifolia, Cassia occidentalis, Desmodium tortuosum, Desmodium adscendens, Desmodium illinoense, Trifolium repens, Pueraria lobata, Vicia angustifolia, Indigofera hirsuta, Indigofera truxillensis, a Vigna sinensis

Oxalidaceae: Oxalis corniculata, Oxalis stricta, and Oxalis oxyptera

Geraniaceae: Geranium carolinense and Erodium cicutarium

Euphorbiaceae: Euphorbia helioscopia, Euphorbia maculata, Euphorbia humistrata, Euphorbia esula, Euphorbia heterophylla, Euphorbia brasiliensis, Acalypha australis, Croton glandulosus, Croton lobatus, Phyllanthus corcovadensis, and Ricinus communis

Malvaceae: Abutilon theophrasti, Sida rhombifolia, Sida cordifolia, Sida spinosa, Sida glaziovii, Sida santaremnensis, Hibiscus trionum, Anoda cristata, and Malvastrum coromandelianum

Onagraceae: Ludwigia epilobioides, Ludwigia octovalvis, Ludwigia decurrens, Oenothera biennis, and Oenothera laciniata

Sterculiaceae: Waltheria indica

Violaceae: Viola arvensis and Viola tricolor

Cucurbitaceae: Sicyos angulatus, Echinocystis lobata, and Momordica charantia

Lythraceae: Ammannia multiflora, Ammannia auriculata, Ammannia coccinea, Lythrum salicaria, and Rotala indica

Elatinaceae: Elatine triandra and Elatine californica

Apiaceae: Oenanthe javanica, Daucus carota, and Conium maculatum

Araliaceae: Hydrocotyle sibthorpioides and Hydrocotyle ranunculoides

Ceratophyllaceae: Ceratophyllum demersum

Cabombaceae: Cabomba caroliniana

Haloragaceae: Myriophyllum aquaticum, Myriophyllum verticillatum, Myriophyllum spicatum, Myriophyllum heterophyllum, and the like.

Sapindaceae: Cardiospermum halicacabum

Primulaceae: Anagallis arvensis

Asclepiadaceae: Asclepias syriaca and Ampelamus albidus

Rubiaceae: Galium aparine, Galium spurium var. echinospermon, Spermacoce latifolia, Richardia brasiliensis, and Borreria alata

Convolvulaceae: Ipomoea nil, Ipomoea hederacea, Ipomoea purpurea, Ipomoea hederacea var. integriuscula, Ipomoea lacunosa, Ipomoea triloba, Ipomoea acuminata, Ipomoea hederifolia, Ipomoea coccinea, Ipomoea quamoclit, Ipomoea grandifolia, Ipomoea aristolochiaefolia, Ipomoea cairica, Convolvulus arvensis, Calystegia hederacea, Calystegia japonica, Merremia hederacea, Merremia aegyptia, Merremia cissoides, and Jacquemontia tamnifolia

Boraginaceae: Myosotis arvensis

Lamiaceae: Lamium purpureum, Lamium amplexicaule, Leonotis nepetaefolia, Hyptis suaveolens, Hyptis lophanta, Leonurus sibiricus, and Stachys arvensis

Solanaceae: Datura stramonium, Solanum nigrum, Solanum americanum, Solanum ptycanthum, Solanum sarrachoides, Solanum rostratum, Solanum aculeatissimum, Solanum sisymbriifolium, Solanum carolinense, Physalis angulata, Physalis subglabrata, and Nicandra physalodes

Scrophulariaceae: Veronica hederaefolia, Veronica persica, Veronica arvensis, Lindernia procumbens, Lindernia dubia, Lindernia angustifolia, Bacopa rotundifolia, Dopatrium junceum, and Gratiola japonica

Plantaginaceae: Plantago asiatica, Plantago lanceolata, Plantago major, and Callitriche palustris

Asteraceae: Xanthium pensylvanicum, Xanthium occidentale, Xanthium italicum, Helianthus annuus, Matricaria chamomilla, Matricaria perforata, Chrysanthemum segetum, Matricaria matricarioides, Artemisia princeps, Artemisia vulgaris, Artemisia verlotorum, Solidago altissima, Taraxacum officinale, Galinsoga ciliata, Galinsoga parviflora, Senecio vulgaris, Senecio brasiliensis, Senecio grisebachii, Conyza bonariensis, Conyza smatrensis, Conyza canadensis, Ambrosia artemisiifolia, Ambrosia trifida, Bidens tripartita, Bidens pilosa, Bidens frondosa, Bidens subalternans, Cirsium arvense, Cirsium vulgare, Silybum marianum, Carduus nutans, Lactuca serriola, Sonchus oleraceus, Sonchus asper, Wedelia glauca, Melampodium perfoliatum, Emilia sonchifolia, Tagetes minuta, Blainvillea latifolia, Tridax procumbens, Porophyllum ruderale, Acanthospermum australe, Acanthospermum hispidum, Cardiospermum halicacabum, Ageratum conyzoides, Eupatorium perfoliatum, Erechtites hieracifolia, Gamochaeta spicata, Gnaphalium spicatum, Jaegeria hirta, Parthenium hysterophorus, Siegesbeckia orientalis, Soliva sessilis, Eclipta prostrata, Eclipta alba, and Centipeda minima

Alismataceae: Sagittaria pygmaea, Sagittaria trifolia, Sagittaria sagittifolia, Sagittaria montevidensis, Sagittaria aginashi, Alisma canaliculatum, and Alisma plantago-aquatica

Limnocharitaceae: Limnocharis flava

Hydrocharitaceae: Limnobium spongia, Hydrilla verticillata, and Najas guadalupensis

Araceae: Pistia stratiotes

Lemnaceae: Lemna aoukikusa, Lemna paucicostata, Lemna aequinoctialis, Spirodela polyrhiza, and Wolffia spp.

Potamogetonaceae: Potamogeton distinctus and Pondweeds (Potamogeton crispus, Potamogeton illinoensis, Stuckenia pectinata, and the like)

Liliaceae: Allium canadense, Allium vineale, and Allium macrostemon

Pontederiaceae: Eichhornia crassipes, Heteranthera limosa, Monochoria korsakowii, and Monochoria vaginalis

Commelinaceae: Commelina communis, Commelina benghalensis, Commelina erecta, and Murdannia keisak

Poaceae: Echinochloa crus-galli, Echinochloa oryzicola, Echinochloa crus-galli var. formosensis, Echinochloa oryzoides, Echinochloa colona, Echinochloa crus-pavonis, Setaria viridis, Setaria faberi, Setaria glauca, Setaria geniculata, Digitaria ciliaris, Digitaria sanguinalis, Digitaria horizontalis, Digitaria insularis, Eleusine indica, Poa annua, Poa trivialis, Poa pratensis, Alopecurus aequalis, Alopecurus myosuroides, Avena fatua, Sorghum halepense, Sorghum vulgare, Agropyron repens, Lolium multiflorum, Lolium perenne, Lolium rigidum, Bromus catharticus, Bromus sterilis, Bromus japonicus, Bromus secalinus, Bromus tectorum, Hordeum jubatum, Aegilops cylindrica, Phalaris arundinacea, Phalaris minor, Apera spica-venti, Panicum dichotomiflorum, Panicum texanum, Panicum maximum, Brachiaria platyphylla, Brachiaria ruziziensis, Brachiaria plantaginea, Brachiaria decumbens, Brachiaria brizantha, Brachiaria humidicola, Cenchrus echinatus, Cenchrus pauciflorus, Eriochloa villosa, Pennisetum setosum, Chloris gayana, Chloris virgata, Eragrostis pilosa, Rhynchelytrum repens, Dactyloctenium aegyptium, Ischaemum rugosum, Isachne globosa, Oryza sativa, Paspalum notatum, Paspalum maritimum, Paspalum distichum, Pennisetum clandestinum, Pennisetum setosum, Rottboellia cochinchinensis, Leptochloa chinensis, Leptochloa fascicularis, Leptochloa filiformis, Leptochloa panicoides, Leersia japonica, Leersia sayanuka, Leersia oryzoides, Glyceria leptorrhiza, Glyceria acutiflora, Glyceria maxima, Agrostis gigantea, Agrostis stolonifera, Cynodon dactylon, Dactylis glomerata, Eremochloa ophiuroides, Festuca arundinacea, Festuca rubra, Imperata cylindrica, Miscanthus sinensis, Panicum virgatum, and Zoysia japonica

Cyperaceae: Cyperus microiria, Cyperus iria, Cyperus compressus, Cyperus difformis, Cyperus flaccidus, Cyperus globosus, Cyperus nipponicus, Cyperus odoratus, Cyperus serotinus, Cyperus rotundus, Cyperus esculentus, Kyllinga gracillima, Kyllinga brevifolia, Fimbristylis miliacea, Fimbristylis dichotoma, Eleocharis acicularis, Eleocharis kuroguwai, Schoenoplectiella hotarui, Schoenoplectiella juncoides, Schoenoplectiella wallichii, Schoenoplectiella mucronatus, Schoenoplectiella triangulatus, Schoenoplectiella nipponicus, Schoenoplectiella triqueter, Bolboschoenus koshevnikovii, and Bolboschoenus fluviatilis

Equisetaceae: Equisetum arvense and Equisetum palustre

Salviniaceae: Salvinia natans

Azollaceae: Azolla japonica and Azolla pinnata

Marsileaceae: Marsilea quadrifolia

Others: filamentous algae (Pithophora, Cladophora), bryophytes, mosses, hornworts, cyanobacteria, ferns, and suckers of perpetual crops (pomaceous fruits, stone fruits, berry fruits, nuts, citrus plants, hops, grapes, and the like).

Regarding the above-mentioned weeds, variations within species are not particularly limited. That is, those having reduced sensitivity (exhibiting resistance) to a specific herbicide are also included. The decrease in sensitivity may be caused by a mutation at a target site (target point mutation) or may be caused by a factor other than the target point mutation (non-target point mutation). The target point mutation includes those in which an amino acid substitution is generated in the protein at the target site due to a mutation in a nucleic acid sequence portion (open reading frame) corresponding to the amino acid sequence of the protein, and those in which the protein at the target site is overexpressed due to a mutation such as deletion of a suppressor sequence in a promoter region, amplification of an enhancer sequence, or an increase in the copy number of a gene. Examples of the non-target point mutation include enhanced metabolism, absorption failure, migration failure, and excretion outside the system. Examples of the factor of the enhanced metabolism include those in which the activity of metabolic enzymes such as cytochrome P450 monooxygenase, arylacylamidase, esterase, and glutathione S-transferase is enhanced. An example of the excretion outside the system includes transport to vacuoles by an ABC transporter.

Examples of the amino acid substitution at the target site include the following.
ALS: A122T, A122V, A122Y, P197S, P197H, P197T, P197R, P197L, P197Q, P197A, P1971, A205V, A205F, D376E, R377H, W574L, W574G, W574M, S653T, S653N, S653I, G654E, or G654D;
ACCase: I1781L, I1781V, I1781T, W1999C, W1999L, A2004V, W2027C, I2041N, I2041V, D2078G or C2088R, G2096A, G2096S;
PPX2: G210Δ, R98L, R98M, R98G, R98H, and G399A; and
EPSP: T102I, P106S, P106A, or P106L.

The weed that can be controlled by the compound of the present invention may have a plurality of amino acid substitutions described above. In this case, the plurality of amino acid substitutions may be the same protein or different proteins. In addition, there may be a plurality of non-target point mutations and target point mutations.

Examples of the weed having a target point mutation include the following weeds.
Amaranthus palmeri having an amino acid substitution of G2104, R98M, R98G, or G399A in PPX2;
Waterhemp having an amino acid substitution of G210A, R98M, R98G, or G399A in PPX2;
Ambrosia artemisiifolia having an amino acid substitution of R98L in PPX2;
Lolium rigidum having an amino acid substitution of R98H in PPX2;
Eleusine indica having an amino acid substitution of T102I, P106S, P106A, or P106L in EPSP;
Lolium rigidum having an amino acid substitution of T102I, P106S, P106A, or P106L in EPSP;
Digitaria insularis having an amino acid substitution of T102I, P106S, P106A, or P106L in EPSP;
Waterhemp having an amino acid substitution of T102I, P106S, P106A, or P106L in EPSP;
Echinochloa colona having an amino acid substitution of T102I, P106S, P106A, or P106L in EPSP;
Echinochloa crus-galli having an amino acid substitution of A122G, A122N, A122V, A122T, A205V, W574L, or W574R in ALS;
Echinochloa oryzicola having an amino acid substitution of P197S or W574L in ALS;
Schoenoplectiella juncoides having an amino acid substitution of P197S, P197T, P197A, P197R, P197H, P197L, D376E, or W574L in ALS;
Schoenoplectiella mucronatus having an amino acid substitution of P197H or W574L in ALS;
Cyperus difformis having an amino acid substitution of P197H in ALS;
Cyperus iria having an amino acid substitution of W574L in ALS; Cyperus rotundus having an amino acid substitution of W574L in ALS;
Monochoria vaginalis var. plantaginea having an amino acid substitution of P197S, P197H, P197L, P197A, P197T, A205V, or D376E in ALS;
Monochoria korsakowii having an amino acid substitution of P197S, P197L, P197A or D376E in ALS;
Sagittaria trifolia having an amino acid substitution of P197S, P197L, P197H, P197T, P197A, or W574L in ALS;
Lindernia dubia having an amino acid substitution of P197A in ALS;
Lindernia dubia having an amino acid substitution of P197S in ALS;
Lindernia procumbens having an amino acid substitution of P197S or P197Q in ALS;
Lindernia angustifolia having an amino acid substitution of P197S or P197Q in ALS;
Echinochloa crus-galli having an amino acid substitution of D2078E in ACCase;
Leptochloa panicoides having an amino acid substitution of W2027C or D2078G in ACCase; and
Leptochloa chinensis having an amino acid substitution of I1781L, W1999C, W2027C, W2027S, or I2041N in ACCase.
Amaranthus palmeri, waterhemp, and Ambrosia artemisiifolia having the above-described target point mutation in PPX2 show resistance to PPO inhibitors such as lactofen, fomesafen, and flumioxazin.

Examples of the weed having a plurality of amino acid substitutions described above include glyphosate-resistant Eleusine indica, Lolium multiflorum, Lolium rigidum, Digitaria insularis, Amaranthus tuberculatus, and Echinochloa colona each having an amino acid substitution of T102I and P106S.

Examples of other weeds include the following weeds.
Amaranthus palmeri, waterhemp, and Kochia scoparia each having reduced sensitivity to glyphosate due to overexpression of EPSP gene;
Echinochloa colona having reduced sensitivity to glyphosate due to increased expression of aldoketo reductase;
resistant Conyza canadensis, Conyza smatrensis, and Conyza bonariensis having reduced sensitivity to glyphosate involving an ABC transporter; and
increased expression of cytochrome P450 monooxygenase results in ACCase inhibitors such as diclofop-methyl, tolalkoxydim, and pinoxadene, ALS inhibitors such as bensulfuron-methyl and penoxsulam, or Echinochloa oryzicola having reduced sensitivity to clomazone.

The composition of the present invention contains the compound of the present invention and an inert carrier. The composition of the present invention is usually used by mixing the compound of the present invention with an inert carrier such as a solid carrier, a liquid carrier, or a gaseous carrier, adding a surfactant or other auxiliaries for formulation as necessary, and formulating the mixture into an aqueous suspension formulation, an oily suspension formulation, an oil formulation, an emulsion, an emulsion formulation, a microemulsion formulation, a microcapsule formulation, a wettable powder, a water dispersible granule, a powder, a granule, a tablet, an aerosol, a resin formulation, or the like. The formulation is not limited to these formulations, and can be formulated and used in the dosage form described in Manual on development and use of FAO and WHO Specifications for pesticides, FAO Plant Production and Protection Papers—271 to 276, prepared by the FAO/WHO Joint Meeting on Pesticide Specifications, 2016, ISSN: 0259-2517. Examples of other auxiliaries for formulation include binders, dispersants, stabilizers, and the like.

These formulations usually contain 0.0001 to 99% by weight of the compound of the present invention. When the compound of the present invention and the present component are contained in one formulation, the compound of the present invention and the present component are usually contained in a total weight ratio of 0.0001 to 99%.

Examples of the solid carrier include fine powders and granules of clay (pyrophyllite clay, kaolin clay, and the like), talc, calcium carbonate, diatomaceous earth, zeolite, bentonite, acidic clay, attapulgite, white carbon, ammonium sulfate, vermiculite, perlite, pumice, silica sand, chemical fertilizers (ammonium sulfate, ammonium phosphate, ammonium nitrate, urea, ammonium chloride, and the like), and resins (polyethylene, polypropylene, polyester, polyurethane, polyamide, polyvinyl chloride, and the like).

Examples of the liquid carrier include water, alcohols (ethanol, cyclohexanol, benzyl alcohol, propylene glycol, polyethylene glycol, and the like), ketones (acetone, cyclohexanone, and the like), aromatic hydrocarbons (xylene, phenylxylylethane, methylnaphthalene, and the like), aliphatic hydrocarbons (hexane, cyclohexane, and the like), esters (ethyl acetate, methyl oleate, propylene carbonate, and the like), nitriles (for example, acetonitrile), ethers (for example, ethylene glycol dimethyl ether), amides (N,N-dimethylformamide, N,N-dimethyloctanamide, and the like), sulfoxides (for example, dimethyl sulfoxide), lactams (N-methylpyrrolidone, N-octylpyrrolidone, and the like), fatty acids (for example, oleic acid), and vegetable oils (for example, soybean oil).

Examples of the gaseous carrier include fluorocarbon, butane gas, LPG (liquefied petroleum gas), dimethyl ether, nitrogen, and carbon dioxide.

Examples of the surfactant include a nonionic surfactant (polyoxyethylene alkyl ether, polyoxyethylene alkyl aryl ether, polyethylene glycol fatty acid ester, and the like) and an anionic surfactant (alkyl sulfonate, alkylaryl sulfonate, alkyl sulfate, and the like).

Examples of other auxiliaries for formulation include binders, dispersants, colorants, stabilizers, and the like, and specific examples thereof include polysaccharides (starch, gum arabic, cellulose derivatives, alginic acid, and the like), lignin derivatives, synthetic water-soluble polymers (polyvinyl alcohol, polyvinyl pyrrolidone, polyacrylic acids, and the like), acidic isopropyl phosphate, and dibutylhydroxytoluene.

In addition, an adjuvant can be used as a component that enhances or assists the efficacy of the compound of the present invention. Specifically, Nimbus (registered trademark), Assist (registered trademark), Aureo (registered trademark), Iharol (registered trademark), Silwet L-77 (registered trademark), BreakThru (registered trademark), Sundancell (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), and BANOLE (registered trademark).

The compound of the present invention can be used in an agricultural land or the like where useful plants are cultivated to control weeds in the agricultural land while suppressing phytotoxicity that causes a problem for the useful plants.

Examples of the useful plants include corn, rice, wheat, barley, rye, oat, sorghum, cotton, soybean, peanut, sugar beet, rapeseed, sunflower, sugar cane, tobacco, and hop;
solanaceae vegetables (eggplant, tomato, bell pepper, capsicum, potato, and the like), cucurbitaceae vegetables (cucumber, squash, zucchini, watermelon, melon, melon, melon, and the like), cruciferae vegetables (radish, turnip, horseradish, collaver, Chinese cabbage, cabbage, mustard, broccoli, cauliflower, and the like), compositae vegetables (burdock, chrysanthemum, artichoke, lettuce, and the like), liliaceae vegetables (green onion, onion, garlic, asparagus, and the like), umbelliferae vegetables (parsley, celery, American boaf, and the like), chenopodiaceae vegetables (spinach, dandruff, and the like), labiatae vegetables (perilla, mint, basil, and the like), fabaceae crops (peas, kidney beans, adzuki bean, broad bean, chick bean, etc.), strawberry, sweet potato, yam, aroid, konjac, ginger, and okra; and
pomaceous fruits (apple, pear, common pear, Chinese quince, quince, and the like), stone fleshy fruits (peach, plum, nectarine, Japanese plum, cherry, apricot, prune, and the like), citrus plants (citrus unshiu, orange, lemon, lime, grapefruit, and the like), nuts (chestnut, walnut, hazel nut, almond, pistachio, cashew nut, macadamia nut, and the like), berry fruits (blueberry, cranberry, blackberry, raspberry, and the like), grape, persimmon, olive, loquat, banana, coffee, date, coconut, oil palm, and the like.

The above useful plants also include PPO inhibitors such as flumioxazin; 4-HPPD inhibitors such as isoxaflutole; ALS inhibitors such as imazethapyr and thifensulfuron-methyl; EPSP synthase inhibitors such as glyphosate; Glutamine synthase inhibitors such as glufosinate; 2,4-D, indole acetic acid-like active agent such as dicamba; ACCase inhibitors such as sethoxydim; and plants to which tolerance to a photosystem II inhibitor such as bromoxynil is imparted by a classical breeding method, genome editing, or a genetic recombination technique.

Examples of the plants to which tolerance is imparted by the classical breeding method include STS soybean tolerant to a sulfonylurea-based ALS inhibitory herbicide such as thifensulfuron-methyl. Similarly, examples of the plants to which tolerance is imparted by the classical breeding method include rice, wheat, corn, rapeseed, and sunflower tolerant to an imidazolinone-based ALS inhibitor, which are already sold under trade names such as Clearfield (registered trademark) and Express (registered trademark). Similarly, examples of the plants to which tolerance is imparted by the classical breeding method include corn and rice tolerant to an ACCase inhibitor, and there are products such as PoastProtected (registered trademark) and Provisia (registered trademark). Similarly, an example of the plant to which tolerance is imparted by the classical breeding method includes Triazine Tolerant rape seed tolerant to a photosystem II inhibitor.

Examples of the plants to which tolerance is imparted by the genetic recombination technique include glyphosate-tolerant soybean, corn, cotton, and rapeseed, which are already sold under trade names such as RoundupReady (registered trademark) and Gly-Tol (registered trademark). Similarly, there is soybean tolerant to glufosinate by the genetic recombination technique, and the soybean is already sold under a trade name such as LibertyLink (registered trademark). There are soybean, corn varieties under the trade name Optimum (registered trademark) GAT (registered trademark) that are tolerant to both glyphosate and ALS inhibitors. There is also soybean tolerant to imidazolinone-based ALS inhibitors by the genetic recombination technique, which has been developed under the name Cultivance. Similarly, as soybean tolerant to both glyphosate and dicamba by the genetic recombination technique, there is a soybean variety having a trade name of RoundupReadyExtend (registered trademark).

A gene encoding aryloxyalkanoate dioxygenase can be introduced to create a crop tolerant to phenoxyacid-based herbicides such as 2,4-D, MCPA, dicloprop, and mecoprop, and aryloxyphenoxypropionic acid-based herbicides such as quizalofop, haloxyfop, fluazifop, diclofop, fenoxaprop, metamifop, cyhalofop, and clodinafop, and there is a soybean variety having a trade name of Enlist E3.

The useful plants include, for example, plants capable of synthesizing selective toxins and the like known in the genus Bacillus using a genetic recombination technique.

Examples of toxins expressed in such genetically modified plants include insecticidal proteins derived from Bacillus cereus and Bacillus popilliae; δ-Endotoxins such as Cry1Ab, Cry1Ac, Cry1F, Cry1Fa2, Cry2Ab, Cry3A, Cry3Bb1, and Cry9C derived from Bacillus thuringiensis, and insecticidal proteins such as VIP1, VIP2, VIP3, and VIP3A; insecticidal protein from nematodes; toxins produced by animals, such as scorpion toxins, spider toxins, bee toxins, or insect-specific neurotoxins; filamentous fungal toxins; plant lectin; agglutinin; trypsin inhibitors, protease inhibitors such as serine protease inhibitors, patatin, cystatin, and papain inhibitors; ribosomal inactivated proteins (RIPs) such as lysine, corn-RIP, abrin, lufin, saporin, and briodine; steroid metabolic enzymes such as 3-hydroxysteroid oxidase, ecdisteroid-UDP-glucosyltransferase, and cholesterol oxidase; ecdysone Inhibitors; HMG-CoA reductase; ion channel inhibitors such as sodium channel inhibitors and calcium channel inhibitors; juvenile hormone esterase; diuretic hormone receptor; stilbene synthase; bibenzyl synthase; chitinase; glucanase, and the like.

In addition, examples of toxins expressed in such genetically modified crops also include hybrid toxins, partially deficient toxins, and modified toxins, such as δ-endotoxin proteins such as Cry1Ab, Cry1Ac, Cry1F, Cry1Fa2, Cry2Ab, Cry3A, Cry3Bb1, Cry9C, Cry34Ab, and Cry35Ab, and insecticidal proteins such as VIP1, VIP2, VIP3, and VIP3A. The hybrid toxins are created by new combinations of different domains of these proteins using recombinant techniques. As a partially deficient toxin, Cry1Ab partially deficient in an amino acid sequence is known. The modified toxin is substituted for one or more of the amino acids of the naturally occurring toxin. Examples of these toxins and recombinant plants from which they can be synthesized are described in EP 0374753 A, WO 93/07278 A, WO 95/34656 A, EP 0427529 A, EP 451878 A, WO 03/052073 A, and the like. Toxins contained in these recombinant plants impart tolerance to, in particular, coleoptera pests, diptera pests, and epidoptera pests to plants.

Examples of these genetically modified plants include Intacta (registered trademark), YieldGard (registered trademark), BollGard (registered trademark), Agrisure (registered trademark) CB/RW, and SmartStax (registered trademark). Examples of the useful plant used in the present invention include plants imparted with tolerance to aphids, such as soybean into which a Rag1 (Resistance Aphid Gene 1) gene has been introduced.

In addition, useful plants used in the present invention include plants to which tolerance to nematodes is imparted using a classical breeding method or a genetic recombination technique. Examples of the genetic recombination technique that provides tolerance to nematodes include RNAi.

The useful plants also include plants imparted with an ability to produce an antipathogenic substance having a selective action using a genetic recombination technique. As an example of the antipathogenic substance, a PR protein and the like are known (PRPs, EP 0392225 A). Such an antipathogenic substance and a genetically modified plant producing the same are described in EP 0392225 A, WO 95/33818 A, EP 0353191 A, and the like. Examples of the antipathogenic substance expressed in such a genetically modified plant include ion channel inhibitors such as sodium channel inhibitors and calcium channel inhibitors (KP1, KP4, KP6 toxins, and the like produced by viruses are known); stilbene synthase; bibenzyl synthase; chitinase; glucanase; PR protein; and peptide antibiotics, antibiotics having a heterocyclic ring, and antipathogenic substances produced by microorganisms such as protein factors (called a plant disease resistance gene and is described in WO 03/000906 A) involved in plant disease resistance.

The useful plants also include plants to which useful traits such as oil feed component modification and amino acid content enhancing traits are imparted using a genetic recombination technique. Examples thereof include VISTIVE (registered trademark) (low-linolenic soybean having a reduced linolenic content). In addition, the useful plants include plants to which a trait or the like for increasing disease tolerance, desiccating stress tolerance, or sugar content is imparted. Examples thereof include DroughtGard (registered trademark) and the like.

Furthermore, a stack variety in which a plurality of the classic herbicide traits or herbicide tolerance genes, insecticidal insect pest resistance genes, antipathogenic substance-producing genes, and useful traits such as oil feed component modification and amino acid content enhancing traits are combined with each other is also included. A method using a genome editing technique instead of genetic recombination is also included.

A method of controlling a weed of the present invention includes a step of applying an effective amount of the compound of the present invention to a weed or a place where the weed grows or will grow. In the method of controlling a weed of the present invention, the compound of the present invention is usually used in an amount of 5 to 5000 g, preferably 10 to 1000 g, per an area of 10000 m² for controlling a weed. When an herbicidal composition containing the compound of the present invention or one or more compounds selected from the compound of the present invention and the present component (hereinafter, referred to as a present herbicidal composition) is applied, a spraying device can be used. Examples of the device used for application include hand sprayers, power spraying devices, puncture sprayers, manned aerial vehicles (such as manned helicopters), unmanned aerial vehicles (radio-controlled helices, drones, and the like), tractors, and planters. In addition, the present herbicidal composition may be applied by hand without using a spraying device.

In the method of controlling a weed of the present invention, the herbicidal composition is usually used in the form of the present herbicidal composition. Examples of the method of controlling a weed of the present invention include a method of performing foliage treatment of the present herbicidal composition on a weed, a method of treating a surface of soil on which a weed grows or will grow, a method of mixing the present herbicidal composition with a soil on which a weed grows, and a method of treating a place on which a weed grows or will grow with surface water of a flooded paddy field.

Examples of the method of applying the present herbicidal composition include a method of uniformly performing a planar treatment or a method of performing a spot treatment of selectively applying the composition. When the present herbicidal composition is applied to some extent to a place where weeds are not grown or a place where weeds are not likely to grow by scattering, transpiration, or the like, the present herbicidal composition is also included in the spot treatment as long as it is not a uniform areal treatment. In addition, in a continuous cultivation area of useful plants, only a case in which all of a place where weeds are growing or a place where weeds are likely to grow are selectively treated is not regarded as a spot treatment. That is, in a case where a part of a cultivation area is planarly treated, or in a case where a part of a place where weeds are growing or a part of a place where weeds are likely to grow is not treated with the present herbicidal composition, if there is a spot treated place in a continuous cultivation area of useful plants, the spot treatment is included. The spot treatment may be performed while avoiding the useful plants, or may be performed based only on the positions of weeds regardless of the positions of the useful plants.

Specific examples of the spot treatment method will be described below. In a cultivation area of useful plants, the spot treatment may be performed by visually spraying the present herbicidal composition using a handheld nozzle or a robotic arm nozzle while a person handling a sprayer is walking or a person handling a sprayer rides on a device travelling on the ground or a flying device. Furthermore, the spot treatment may be performed by mapping a place where weeds are growing or are likely to grow in advance, and applying the compound of the present invention or the present herbicidal composition based on map information. In the spraying based on the map information, in addition to the above method, during traveling or flight of the spraying device, the spot treatment may be performed by automatically or manually opening and closing a nozzle on a boom or a robot arm nozzle based on the position information (obtained by GPS or the like) of the spraying device and the map information. The map information may be created on the basis of image information captured by a manned or unmanned flying object or the like, or may be created visually by an observer walking on the ground, an observer riding on a device traveling on the ground, or an observer riding on a flying device. Further, the spraying device that travels or flies may have a function of detecting a place where weeds are growing or a place where weeds are likely to grow, and the spot treatment may be performed by the boom, the robot arm, or the like while performing real-time mapping. Such a technique is described in Patent Literature (for example, WO 2018001893 A, WO 2018036909 A) and Non-Patent Literature (for example, Crop Protection 26, 270-277, Weed Technology 17, 711-717, Applied Engineering in Agriculture. 30, 143-152). These techniques are a form of emerging agriculture called precision agriculture, smart agriculture, digital agriculture, or the like, and a non-uniform spraying manner generated by the spot treatment is also called variable rate application (VRA) as a term in the emerging agriculture.

The place where the weeds may grow may be estimated based on the fact that the weeds have formed vegetation patches in the past growth period, or may be estimated from the distribution of buried seeds. The distribution of the buried seeds may be investigated by soil sampling or estimated by remote sensing.

### EXAMPLES

Hereinafter, the present invention will be described in more detail with reference to production examples, reference production examples, formulation examples, test examples, and the like, but the present invention is not limited only to these examples.

In the present specification, Me represents a methyl group.

First, production examples of the compound of the present invention and production intermediates thereof will be shown.

### Reference Production Example 1

A mixture including 7.13 g of 2,4-dibromobutyric acid methyl ester and 10 mL of DMF was added to a mixture including 5.00 g of 2-chloro-4-fluoro-5-nitrophenol, 3.97 g of potassium carbonate, and 20 mL of DMF under ice cooling, and the mixture was stirred at room temperature for eight hours. Water was added to the obtained mixture, then MTBE was added thereto, and the mixture was extracted using MTBE. After an obtained organic layer was washed using water, the obtained organic layer was washed using saturated saline, was dried using anhydrous magnesium sulfate, and was concentrated under reduced pressure. An obtained residue was applied to a silica gel column chromatography (hexane:ethyl acetate=1:1) to obtain 8.39 g of a compound (A3-1) represented by the following formula.

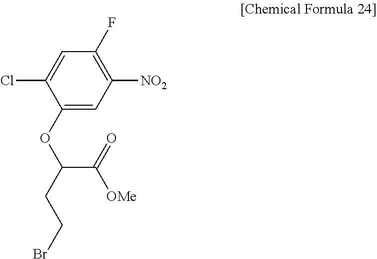 [SMILES: COC(=O)C(CCBr)Oc1cc([N+](=O)[O-])c(F)cc1Cl]

Compound (A3-1): ¹H-NMR (CDCl₃) δ:7.66 (1H, d), 7.38 (1H, d), 3.76 (3H, s), 1.76-1.73 (2H, m), 1.45-1.41 (2H, m).

### Reference Production Example 2

23 mL of a THF solution (1 mol/L) of potassium t-butoxide was added dropwise to a mixture including 8.39 g of the compound (A3-1) and 23 mL of THF at −40° C., and the mixture was stirred at room temperature for eight hours. After diluted hydrochloric acid was added to the obtained mixture under ice cooling, ethyl acetate was added to the obtained mixture, and the mixture was extracted using ethyl acetate. After an obtained organic layer was washed using water, the obtained organic layer was washed using saturated saline, was dried using anhydrous sodium sulfate, and was concentrated under reduced pressure. An obtained residue was applied to a silica gel column chromatography (hexane:ethyl acetate=1:1) to obtain 3.03 g of a compound (A4-1) represented by the following formula.

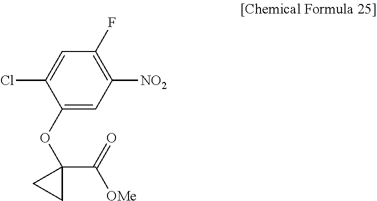 [SMILES: COC(=O)C1(Oc2cc([N+](=O)[O-])c(F)cc2Cl)CC1]

Compound (A4-1): ¹H-NMR (CDCl₃) δ:7.59 (1H, d), 7.40 (1H, d), 4.99 (1H, m), 3.83 (3H, s), 3.73-3.61 (2H, m), 2.66-2.49 (2H, m).

### Reference Production Example 3

A mixture including 10.54 g of the compound (A4-1) and 36 mL of ethyl acetate was added dropwise to a mixture including 20.33 g of iron powder, 45 mL of water, and 45 mL of acetic acid at 60° C., and the mixture was stirred at 60° C. for eight hours. Saturated saline and ethyl acetate were added to the obtained mixture, and the mixture was stirred at 60° C. for 30 minutes, thereby extracting the mixture using ethyl acetate. After an obtained organic layer was washed using water, the obtained organic layer was washed using saturated saline, was dried using anhydrous sodium sulfate, and was concentrated under reduced pressure. An obtained residue was applied to a silica gel column chromatography (hexane:ethyl acetate=1:1) to obtain 5.33 g of a compound (A5-1) represented by the following formula.

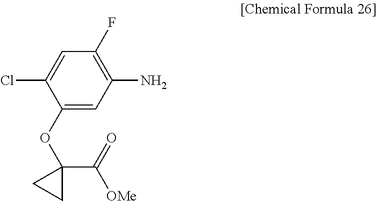 [SMILES: COC(=O)C1(Oc2cc(N)c(F)cc2Cl)CC1]

Compound (A5-1): ¹H-NMR (CDCl₃) δ:7.00 (1H, m), 6.45 (1H, d), 3.78-3.71 (5H, m), 1.63-1.59 (2H, m), 1.39-1.35 (2H, m).

### Reference Production Example 4

A mixture including 28.04 g of the compound (A3-1) and 75 mL of ethyl acetate was added dropwise to a mixture including 42.27 g of iron powder, 75 mL of water, and 75 mL of acetic acid at 60° C., and the mixture was stirred at 60° C. for eight hours. Saturated saline and ethyl acetate were added to the obtained mixture, and the mixture was stirred at 60° C. for 30 minutes. Thereafter, the mixture was extracted using ethyl acetate. After an obtained organic layer was washed using water, the obtained organic layer was washed using saturated saline, was dried using anhydrous sodium sulfate, and was concentrated under reduced pressure. An obtained residue was applied to a silica gel column chromatography (hexane:ethyl acetate=1:1) to obtain 19.40 g of a compound (A6-1) represented by the following formula.

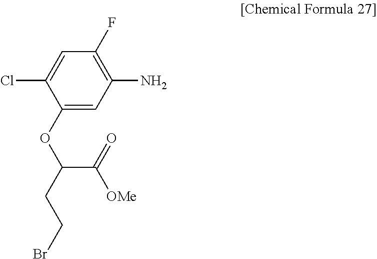 [SMILES: COC(=O)C(CCBr)Oc1cc(N)c(F)cc1Cl]

Compound (A6-1): ¹H-NMR (CDCl₃) δ:7.02 (1H, d), 6.37 (1H, d), 4.77 (1H, m), 3.79 (3H, s), 3.71 (1H, m), 3.63 (1H, m), 2.56 (1H, m), 2.44 (1H, m).

### Reference Production Example 5

A mixture including 19.4 g of compound (A6-1) and 30 mL of THF was added dropwise to a mixture including 6.4 g of potassium t-butoxide and 30 mL of THF at −5° C., and the mixture was stirred under a reflux condition for eight hours. After a saturated aqueous sodium bicarbonate solution was added to the obtained mixture, ethyl acetate was added to the mixture, and the mixture was extracted using ethyl acetate. An obtained organic layer was washed using saturated saline, was dried using anhydrous sodium sulfate, and was concentrated under reduced pressure. An obtained residue was applied to a silica gel column chromatography (hexane:ethyl acetate=1:1), thereby obtaining 8.26 g of a compound (A5-1).

### Reference Production Example 6

3 mL of an aqueous sodium hydroxide solution (3 mol/L) was added to a mixture including 1.73 g of the compound (A4-1), 6 mL of THE, and 3 mL of methanol under ice cooling, and the mixture was stirred under a reflux condition for eight hours. 3 mL of an aqueous lithium hydroxide solution (3 mol/L) was added to the obtained mixture at room temperature, and the mixture was stirred under a reflux condition for eight hours. After water was added to the obtained mixture at room temperature, MTBE was added to the mixture, and the mixture was washed using MTBE. After diluted hydrochloric acid was added to an obtained aqueous layer to make it acidic, ethyl acetate was added to the obtained aqueous layer, and the layer was extracted using ethyl acetate. After an obtained organic layer was washed using saturated saline, the organic layer was dried using anhydrous sodium sulfate and was concentrated under reduced pressure, thereby obtaining 1.65 g of a mixture containing a compound (A11-1) represented by the following formula.

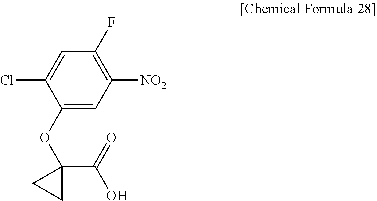 [SMILES: O=C(O)C1(Oc2cc([N+](=O)[O-])c(F)cc2Cl)CC1]

### Compound (A11-1)

### Reference Production Example 7

A mixture including 1.01 g of methyl bromoacetate and 5 mL of DMF was added to a mixture including 1.65 g of a mixture containing the compound (A11-1) obtained in reference production example 6, 1 g of potassium carbonate, and 10 mL of DMF, and the mixture was stirred at 50° C. for eight hours. Water was added to the obtained mixture, then MTBE was added thereto, and the mixture was extracted using MTBE. After an obtained organic layer was washed using water, the obtained organic layer was washed using saturated saline, was dried using anhydrous magnesium sulfate, and was concentrated under reduced pressure. An obtained residue was applied to a silica gel column chromatography (hexane:ethyl acetate=1:1) to obtain 1.31 g of a compound (A12-1) represented by the following formula.

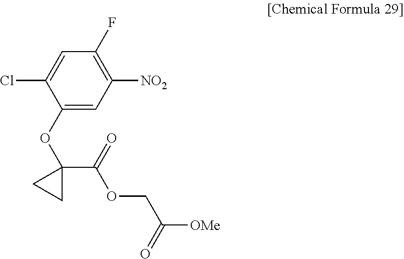 [SMILES: COC(=O)COC(=O)C1(Oc2cc([N+](=O)[O-])c(F)cc2Cl)CC1]

Compound (A12-1): ¹H-NMR (CDCl₃) δ:7.84 (1H, d), 6.36 (1H, d), 4.69 (2H, s), 3.77 (3H, s), 1.86-1.82 (2H, m), 1.51-1.47 (2H, m).

### Reference Production Example 8

A mixture including 1.31 g of the compound (A12-1) and 10 mL of toluene was added dropwise to a mixture including 2.11 g of iron powder, 0.07 g of iron (III) chloride, and 10 mL of water under a reflux condition, and the mixture was stirred under the reflux condition for eight hours. Toluene was added to the obtained mixture, and the mixture was extracted using toluene. After an obtained organic layer was washed using water, the obtained organic layer was washed using saturated saline, was dried using anhydrous sodium sulfate, and was concentrated under reduced pressure. An obtained residue was applied to a silica gel column chromatography (hexane:ethyl acetate=1:1) to obtain 1.04 g of a compound (A13-1) represented by the following formula.

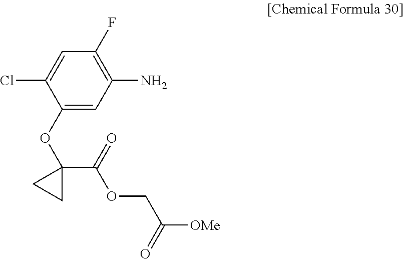 [SMILES: COC(=O)COC(=O)C1(Oc2cc(N)c(F)cc2Cl)CC1]

Compound (A13-1): ¹H-NMR (CDCl₃) δ:6.99 (1H, d), 6.75 (1H, d), 4.70 (2H, s), 3.79 (2H, br s), 3.77 (3H, s), 1.71-1.68 (2H, m), 1.44-1.41 (2H, m).

### Production Example 1

A mixture including 7.6 g of triphosgene and 20 mL of toluene was added dropwise to a mixture including 6.4 g of the compound (A5-1) obtained in reference production example 3 and 30 mL of toluene at room temperature, and the mixture was stirred under a reflux condition for eight hours. After stirring, the mixture was concentrated under reduced pressure, 3.21 g of 1,3-dimethylthiourea, 3.89 g of triethylamine, 6.24 g of 1,1′-carbonyldiimidazole, and 50 mL of toluene were added to the obtained residue, and the obtained mixture was stirred at 80° C. for eight hours. After a saturated aqueous ammonium chloride solution was added to the obtained mixture, toluene was added to the mixture, and the mixture was extracted using toluene. After an obtained organic layer was washed using water, the obtained organic layer was washed using saturated saline, was dried using anhydrous sodium sulfate, and was concentrated under reduced pressure. An obtained residue was applied to a silica gel column chromatography (hexane:ethyl acetate=1:1) to obtain 6.63 g of a compound of the present invention (1-1-1) represented by the following formula.

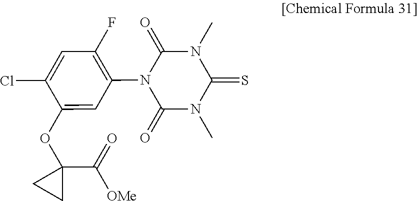 [SMILES: COC(=O)C1(Oc2cc(-n3c(=O)n(C)c(=S)n(C)c3=O)c(F)cc2Cl)CC1]

Compound of the Present Invention (1-1-1): ¹H-NMR (CDCl₃) δ:7.31 (1H, d), 6.91 (1H, d), 3.77 (6H, s), 3.74 (3H, s), 1.67-1.63 (2H, m), 1.43-1.40 (2H, m).

### Production Example 2

A mixture including 6.63 g of the compound of the present invention (1-1-1), 80 mL of 1,4-dioxane, and 80 mL of hydrochloric acid (6 mol/L) was stirred under a reflux condition for eight hours. Ethyl acetate was added to the obtained mixture, and the mixture was extracted using ethyl acetate. After an obtained organic layer was washed using water, the obtained organic layer was washed using saturated saline, was dried using anhydrous sodium sulfate, and was concentrated under reduced pressure. An obtained residue was applied to a silica gel column chromatography (hexane:ethyl acetate=1:1) to obtain 5.46 g of a compound of the present invention (I-2-1) represented by the following formula.

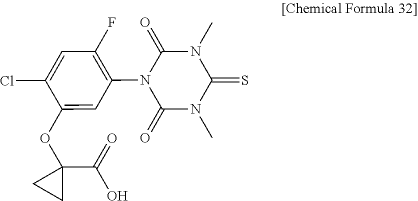 [SMILES: Cn1c(=S)n(C)c(=O)n(-c2cc(OC3(C(=O)O)CC3)c(Cl)cc2F)c1=O]

Compound of the Present Invention (1-2-1): ¹H-NMR (CDCl₃) δ:7.33 (1H, d), 6.99 (1H, d), 3.77 (6H, s), 1.72-1.68 (2H, m), 1.50-1.47 (2H, m).

### Production Example 3

A mixture including 0.1 g of ethyl bromoacetate and 5 mL of DMF was added to a mixture including 0.21 g of the compound of the present invention (I-2-1), 0.09 g of potassium carbonate, and 5 mL of DMF under ice cooling, and the mixture was stirred at room temperature for eight hours. Water was added to the obtained mixture, then MTBE was added thereto, and the mixture was extracted using MTBE. After an obtained organic layer was washed using water, the obtained organic layer was washed using saturated saline, was dried using anhydrous magnesium sulfate, and was concentrated under reduced pressure. An obtained residue was applied to a silica gel column chromatography (hexane:ethyl acetate=1:1) to obtain 0.2 g of a compound of the present invention (1-14) represented by the following formula.

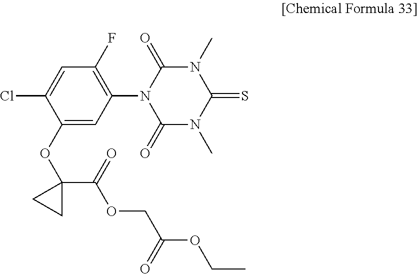 [SMILES: CCOC(=O)COC(=O)C1(Oc2cc(-n3c(=O)n(C)c(=S)n(C)c3=O)c(F)cc2Cl)CC1]

Compound of the Present Invention (I-14): ¹H-NMR (CDCl₃) δ:7.23-7.26 (2H, m), 4.66 (2H, s), 4.14 (2H, q), 3.76 (6H, s), 1.74-1.70 (2H, m), 1.48-1.45 (2H, m), 1.25 (3H, t).

### Production Example 4

0.11 g of 1-[bis(dimethylamino)methylene]-1H-1,2,3-triazolo[4,5-b]pyridinium 3-oxide hexafluorophosphate was added to a mixture including 0.10 g of the compound of the present invention (I-2-1), 0.11 ml of diisopropylethylamine, 31 mg of a 50 wt % aqueous dimethylamine solution, and 5.5 mL of DMF at room temperature, and the mixture was stirred at room temperature for two hours. After diluted hydrochloric acid was added to the obtained mixture at room temperature, MTBE was added to the mixture, and the mixture was extracted using MTBE. After an obtained organic layer was washed using water, the obtained organic layer was washed using saturated saline, was dried using anhydrous magnesium sulfate, and was concentrated under reduced pressure. An obtained residue was applied to a silica gel column chromatography (hexane:ethyl acetate=1:1) to obtain 75 mg of a compound of the present invention (1-10) represented by the following formula.

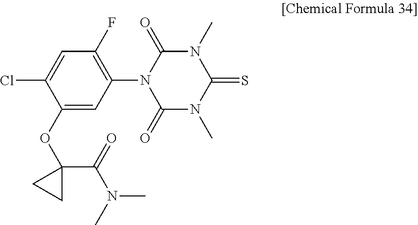 [SMILES: CN(C)C(=O)C1(Oc2cc(-n3c(=O)n(C)c(=S)n(C)c3=O)c(F)cc2Cl)CC1]

Compound of the Present Invention (I-10): ¹H-NMR (CDCl₃) δ:7.29 (1H, d), 7.23 (1H, d), 6.36 (1H, m), 3.76 (6H, s), 3.24 (3H, s), 2.90 (3H, s), 1.48-1.45 (2H, m), 1.24-1.21 (2H, m).

### Production Example 5

0.10 mL of oxalyl chloride was added to a mixture including 0.30 g of the compound of the present invention (I-2-1), 1 drop of DMF, and 6.0 mL of chloroform at room temperature, and the mixture was stirred at room temperature for one hour. After a mixture including 87 mg of ethyl-1-hydroxy-1-cyclopropanecarboxylate and 3.0 mL of chloroform was added to the obtained mixture at room temperature, 0.16 mL of triethylamine was added to the mixture, and the mixture was stirred at room temperature for one hour. After diluted hydrochloric acid was added to the obtained mixture under ice cooling, chloroform was added to the mixture, and the mixture was extracted using chloroform. After an obtained organic layer was washed using water, the obtained organic layer was washed using saturated saline, was dried using anhydrous magnesium sulfate, and was concentrated under reduced pressure. An obtained residue was applied to a silica gel column chromatography (hexane:ethyl acetate=1:1) to obtain 123 mg of a compound of the present invention (1-23) represented by the following formula.

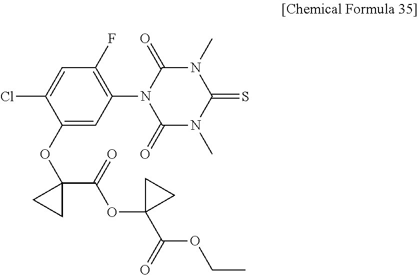 [SMILES: CCOC(=O)C1(OC(=O)C2(Oc3cc(-n4c(=O)n(C)c(=S)n(C)c4=O)c(F)cc3Cl)CC2)CC1]

Compound of the Present Invention (I-23): ¹H-NMR (CDCl₃) δ:7.29 (1H, d), 7.24 (1H, d), 4.10 (2H, q), 3.77 (6H, s), 1.70-1.67 (2H, m), 1.52-1.48 (2H, m), 1.45-1.42 (2H, m), 1.22-1.17 (5H, m).

### Production Example 6

A mixture including 0.30 g of the compound (A13-1) and 5 mL of toluene was added dropwise to a mixture including 0.19 g of diphosgene and 5 mL of toluene at room temperature, and the mixture was stirred under a reflux condition for eight hours. After stirring, the mixture was concentrated under reduced pressure, and a mixture including 0.12 g of 1,3-dimethylthiourea, 0.15 g of triethylamine, and 5 mL of toluene was added to an obtained residue. A mixture including 0.24 g of 1,1′-carbonyldiimidazole and 5 mL of toluene was added dropwise to the obtained mixture under a reflux condition, and the obtained mixture was stirred under the reflux condition for eight hours. After water was added to the obtained mixture, toluene was added to the mixture, and the mixture was extracted using toluene. After an obtained organic layer was washed using water, the obtained organic layer was washed using saturated saline, was dried using anhydrous sodium sulfate, and was concentrated under reduced pressure. An obtained residue was applied to a silica gel column chromatography (hexane:ethyl acetate-1:1) to obtain 0.15 g of a compound of the present invention (I-13) represented by the following formula.

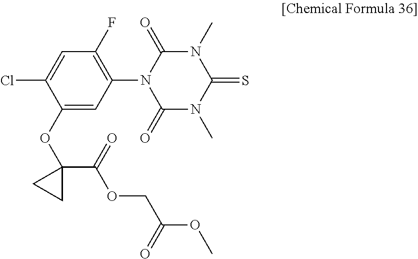 [SMILES: COC(=O)COC(=O)C1(Oc2cc(-n3c(=O)n(C)c(=S)n(C)c3=O)c(F)cc2Cl)CC1]

Compound of the Present Invention (I-13): ¹H-NMR (CDCl₃) δ:7.29 (1H, d), 7.25 (1H, d), 4.68 (2H, s), 3.77 (6H, s), 3.70 (3H, s), 1.74-1.70 (2H, m), 1.49-1.45 (2H, m).

### Production Example 7

A compound produced according to the production method, the method described in the production example, or the like and physical property values thereof are shown below.

### Formula (I)

In a compound represented by

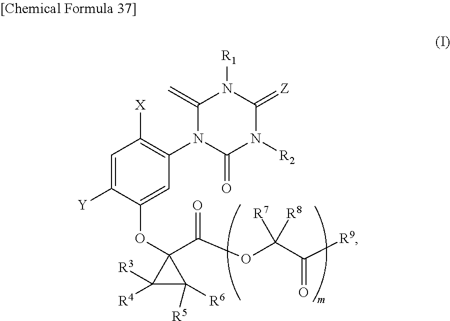

a compound wherein a combination of R¹, R², R³, R⁴, R⁵, R⁶, R⁷, R⁸, R⁹, X, Y, Z, and m is any one of the combinations shown in [Table L2] and [Table L3].

Comp described in [Table L2] and [Table L3] means the compound number of the present invention.

R⁹ represents any one of substituent numbers 1 to 36 described in [Table L1].

For example, a compound in which Comp (compound number of present invention) described in [Table L2] is I-7, that is, the compound of the present invention (I-7) means a compound in which R⁹ is a group, the substituent number of which is 17, described in [Table L1], R¹ and R² are each a methyl group, R³, R⁴, R⁵, and R⁶ are each a hydrogen atom, X is a fluorine atom, Y is a chlorine atom, Z is a sulfur atom, and m is 0. The compound of the present invention (I-7) is specifically a compound having the following structure.

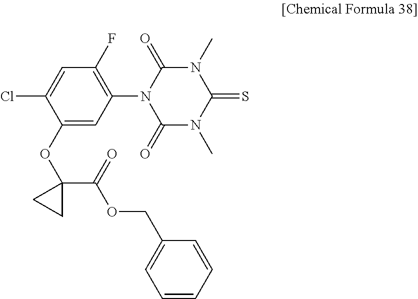 [SMILES: Cn1c(=S)n(C)c(=O)n(-c2cc(OC3(C(=O)OCc4ccccc4)CC3)c(Cl)cc2F)c1=O]

TABLE 1

| Substituent | | Substituent | | Substituent | |
|---|---|---|---|---|---|
| number | R⁹ | number | R⁹ | number | R⁹ |
| 1 | 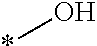 [SMILES: *O] | 13 | 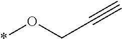 [SMILES: *OCC#C] | 25 | 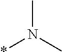 [SMILES: *N(C)C] |
| 2 | 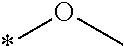 [SMILES: *OC] | 14 | 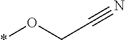 [SMILES: *OCC#N] | 26 | 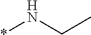 [SMILES: *NCC] |
| 3 | 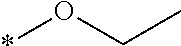 [SMILES: *OCC] | 15 | 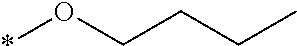 [SMILES: *OCCCC] | 27 | 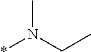 [SMILES: *N(C)CC] |
| 4 | 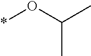 [SMILES: *OC(C)C] | 16 | 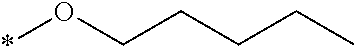 [SMILES: *OCCCCC] | 28 | 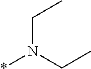 [SMILES: *N(CC)CC] |
| 5 | 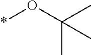 [SMILES: *OC(C)(C)C] | 17 | 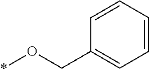 [SMILES: *OCc1ccccc1] | 29 | 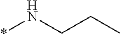 [SMILES: *NCCC] |
| 6 | 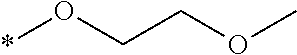 [SMILES: *OCCOC] | 18 | 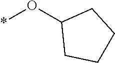 [SMILES: *OC1CCCC1] | 30 | 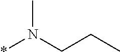 [SMILES: *N(C)CCC] |
| 7 | 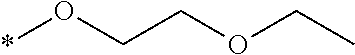 [SMILES: *OCCOCC] | 19 | 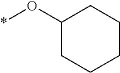 [SMILES: *OC1CCCCC1] | 31 | 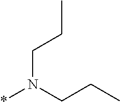 [SMILES: *N(CCC)CCC] |
| 8 | 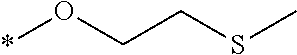 [SMILES: *OCCSC] | 20 | 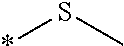 [SMILES: *SC] | 32 | 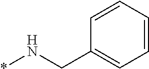 [SMILES: *NCc1ccccc1] |
| 9 | 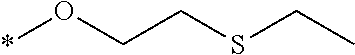 [SMILES: *OCCSCC] | 21 | 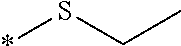 [SMILES: *SCC] | | |
| 10 | 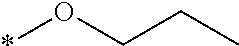 [SMILES: *OCCC] | 22 | 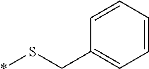 [SMILES: *SCc1ccccc1] | | |
| 11 | 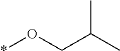 [SMILES: *OCC(C)C] | 23 | 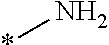 [SMILES: *N] | | |
| 12 | 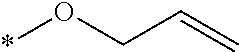 [SMILES: *OCC=C] | 24 | 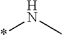 [SMILES: *NC] | | |

TABLE L2

| Comp [symbol] | R [symbol] | R [symbol] | R [symbol] | R [symbol] | R [symbol] | R [symbol] | R [symbol] | X | Y | Z | m |
|---|---|---|---|---|---|---|---|---|---|---|---|
| I-1-1 | Me | Me | H | H | H | H | 2 | F | Cl | S | 0 |
| I-2-1 | Me | Me | H | H | H | H | 1 | F | Cl | S | 0 |
| I-1-2 | Me | Me | H | H | H | H | 3 | F | Cl | S | 0 |
| I-4 | Me | Me | H | H | H | H | 10 | F | Cl | S | 0 |
| I-5 | Me | Me | H | H | H | H | 12 | F | Cl | S | 0 |
| I-6 | Me | Me | H | H | H | H | 13 | F | Cl | S | 0 |
| I-7 | Me | Me | H | H | H | H | 17 | F | Cl | S | 0 |
| I-8 | Me | Me | H | H | H | H | 24 | F | Cl | S | 0 |
| I-9 | Me | Me | H | H | H | H | 26 | F | Cl | S | 0 |
| I-10 | Me | Me | H | H | H | H | 25 | F | Cl | S | 0 |
| I-11 | Me | Me | H | H | H | H | 28 | F | Cl | S | 0 |

[symbol]

indicates data missing or illegible when filed

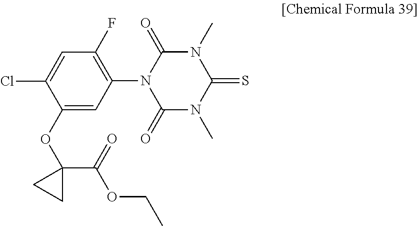 [SMILES: CCOC(=O)C1(Oc2cc(-n3c(=O)n(C)c(=S)n(C)c3=O)c(F)cc2Cl)CC1]

Compound of the Present Invention (I-1-2): ¹H-NMR (CDCl₃) δ:7.31 (1H, d), 6.93 (1H, d), 4.19 (2H, q), 3.77 (6H, s), 1.67-1.62 (2H, m), 1.42-1.40 (2H, m), 1.19 (3H, t).

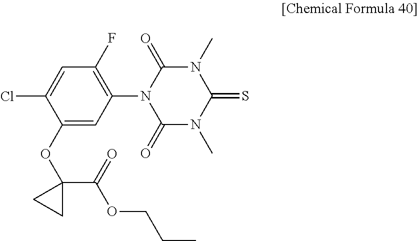 [SMILES: CCCOC(=O)C1(Oc2cc(-n3c(=O)n(C)c(=S)n(C)c3=O)c(F)cc2Cl)CC1]

Compound of the Present Invention (I-4): ¹H-NMR (CDCl₃) δ:7.31 (1H, d), 6.94 (1H, d), 4.08 (2H, t), 3.77 (6H, s), 1.65-1.62 (2H, m), 1.60-1.52 (2H, m), 1.43-1.39 (2H, m), 0.80 (3H, t).

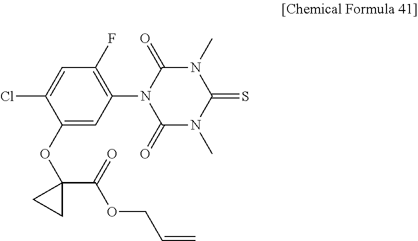 [SMILES: C=CCOC(=O)C1(Oc2cc(-n3c(=O)n(C)c(=S)n(C)c3=O)c(F)cc2Cl)CC1]

Compound of the Present Invention (I-5): ¹H-NMR (CDCl₃) δ:7.31 (1H, d), 6.94 (1H, d), 5.82 (1H, m), 5.25-5.16 (2H, m), 4.64-4.62 (2H, m), 3.77 (6H, s), 1.67-1.64 (2H, m), 1.44-1.41 (2H, m).

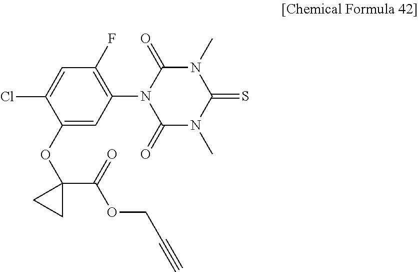 [SMILES: C#CCOC(=O)C1(Oc2cc(-n3c(=O)n(C)c(=S)n(C)c3=O)c(F)cc2Cl)CC1]

Compound of the Present Invention (I-6): ¹H-NMR (CDCl₃) δ:7.31 (1H, d), 6.93 (1H, d), 4.73 (2H, d), 3.77 (6H, s), 2.44 (1H, m), 1.71-1.67 (2H, m), 1.47-1.44 (2H, m).

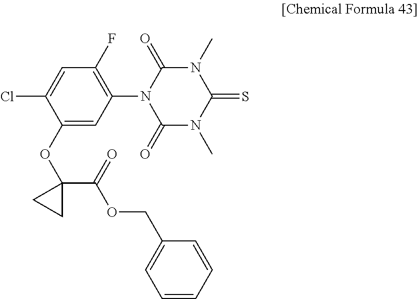 [SMILES: Cn1c(=S)n(C)c(=O)n(-c2cc(OC3(C(=O)OCc4ccccc4)CC3)c(Cl)cc2F)c1=O]

Compound of the Present Invention (I-7): ¹H-NMR (CDCl₃) δ:7.32-7.27 (4H, m), 7.24-7.21 (2H, m), 6.92 (1H, d), 5.16 (2H, s), 3.78 (6H, s) 1.67-1.64 (2H, m), 1.44-1.41 (2H, m).

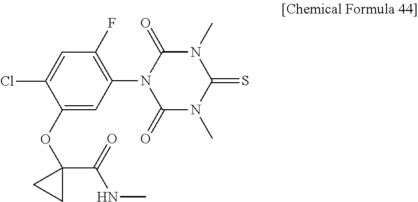 [SMILES: CNC(=O)C1(Oc2cc(-n3c(=O)n(C)c(=S)n(C)c3=O)c(F)cc2Cl)CC1]

Compound of the Present Invention (I-8): ¹H-NMR (CDCl₃) δ:7.33 (1H, d), 6.96 (1H, d), 6.36 (1H, m), 3.77 (6H, s), 2.85 (3H, d), 1.67-1.63 (2H, m), 1.25-1.22 (2H, m).

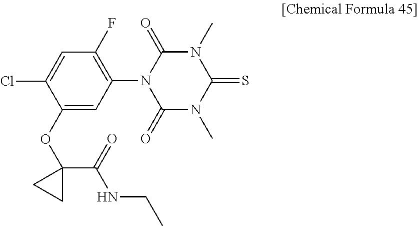 [SMILES: CCNC(=O)C1(Oc2cc(-n3c(=O)n(C)c(=S)n(C)c3=O)c(F)cc2Cl)CC1]

Compound of the Present Invention (I-9): ¹H-NMR (CDCl₃) δ:7.33 (1H, d), 6.97 (1H, d), 6.34 (1H, m), 3.77 (6H, s), 3.36-3.29 (2H, m), 1.66-1.62 (2H, m), 1.24-1.21 (2H, m), 1.11 (3H, t).

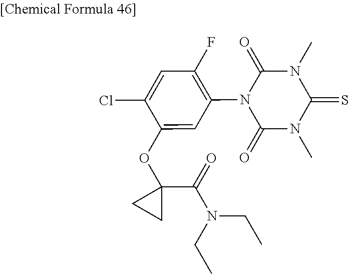 [SMILES: CCN(CC)C(=O)C1(Oc2cc(-n3c(=O)n(C)c(=S)n(C)c3=O)c(F)cc2Cl)CC1]

Compound of the Present Invention (I-11): ¹H-NMR (CDCl₃) δ:7.29-7.26 (2H, m), 3.75 (6H, s), 3.68 (2H, q), 3.30 (2H, q), 1.46-1.43 (2H, m), 1.22-1.18 (2H, m), 1.14 (3H, t), 1.03 (3H, t).

TABLE L3

| Comp [symbol] | R [symbol] | R [symbol] | R [symbol] | R [symbol] | R [symbol] | R [symbol] | R [symbol] | R [symbol] | R [symbol] | X | Y | Z | m |
|---|---|---|---|---|---|---|---|---|---|---|---|---|---|
| I-12 | Me | Me | H | H | H | H | H | H | 1 | F | Cl | S | 1 |
| I-13 | Me | Me | H | H | H | H | H | H | 2 | F | Cl | S | 1 |
| I-14 | Me | Me | H | H | H | H | H | H | 3 | F | Cl | S | 1 |
| I-15 | Me | Me | H | H | H | H | H | H | 10 | F | Cl | S | 1 |
| I-16 | Me | Me | H | H | H | H | H | H | 15 | F | Cl | S | 1 |
| I-17 | Me | Me | H | H | H | H | H | H | 5 | F | Cl | S | 1 |
| I-18 | Me | Me | H | H | H | H | H | H | 12 | F | Cl | S | 1 |
| I-19 | Me | Me | H | H | H | H | H | H | 13 | F | Cl | S | 1 |
| I-20 | Me | Me | H | H | H | H | H | H | 17 | F | Cl | S | 1 |
| I-21 | Me | Me | H | H | H | H | Me | H | 3 | F | Cl | S | 1 |
| I-22 | Me | Me | H | H | H | H | Me | Me | 3 | F | Cl | S | 1 |
| I-23 | Me | Me | H | H | H | H | —(CH₂CH₂) [symbol] — | | 3 | F | Cl | S | 1 |
| I-24 | Me | Me | H | H | H | H | H | H | 25 | F | Cl | S | 1 |
| I-25 | Me | Me | H | H | H | H | H | H | 28 | F | Cl | S | 1 |

[symbol]

indicates data missing or illegible when filed

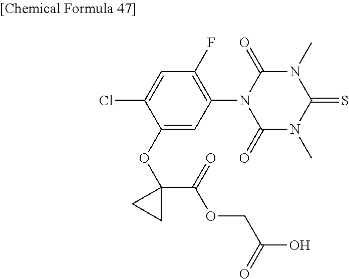 [SMILES: Cn1c(=S)n(C)c(=O)n(-c2cc(OC3(C(=O)OCC(=O)O)CC3)c(Cl)cc2F)c1=O]

Compound of the Present Invention (I-12): ¹H-NMR (CD₃OD) δ:7.42 (1H, d), 7.40 (1H, d), 4.65 (2H, s), 3.73 (6H, s), 1.71-1.68 (2H, m), 1.43-1.40 (2H, m).

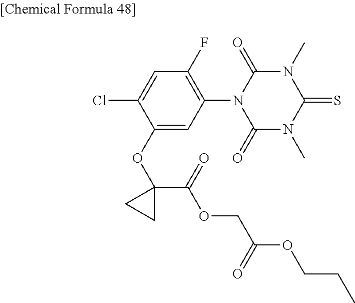 [SMILES: CCCOC(=O)COC(=O)C1(Oc2cc(-n3c(=O)n(C)c(=S)n(C)c3=O)c(F)cc2Cl)CC1]

Compound of the Present Invention (I-15): ¹H-NMR (CDCl₃) δ:7.30-7.26 (2H, m), 4.67 (2H, s), 4.04 (2H, t), 3.77 (6H, s), 1.74-1.70 (2H, m), 1.68-1.59 (2H, m), 1.48-1.45 (2H, m), 0.92 (3H, t).

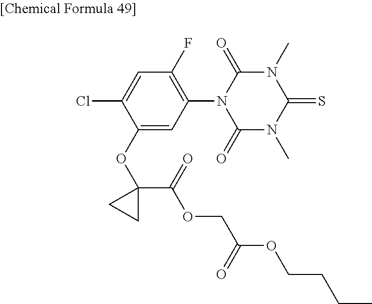 [SMILES: CCCCOC(=O)COC(=O)C1(Oc2cc(-n3c(=O)n(C)c(=S)n(C)c3=O)c(F)cc2Cl)CC1]

Compound of the Present Invention (I-16): ¹H-NMR (CDCl₃) δ:7.29-7.26 (2H, m), 4.66 (2H, s), 4.08 (2H, t), 3.76 (6H, s), 1.74-1.70 (2H, m), 1.63-1.56 (2H, m), 1.48-1.44 (2H, m), 1.40-1.30 (2H, m), 0.92 (3H, t).

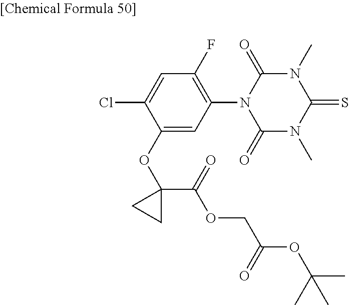 [SMILES: Cn1c(=S)n(C)c(=O)n(-c2cc(OC3(C(=O)OCC(=O)OC(C)(C)C)CC3)c(Cl)cc2F)c1=O]

Compound of the Present Invention (I-17): ¹H-NMR (CDCl₃) δ:7.31 (1H, d), 7.28 (1H, d), 4.55 (2H, s), 3.76 (6H, s), 1.74-1.70 (2H, m), 1.46-1.43 (2H, m), 1.41 (9H, s).

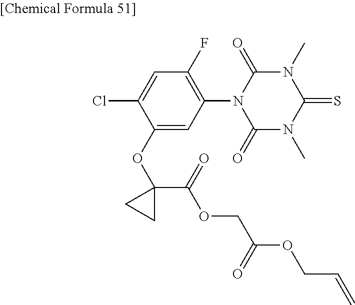 [SMILES: C=CCOC(=O)COC(=O)C1(Oc2cc(-n3c(=O)n(C)c(=S)n(C)c3=O)c(F)cc2Cl)CC1]

Compound of the Present Invention (I-18): ¹H-NMR (CDCl₃) δ:7.29 (1H, d), 7.24 (1H, d), 5.86 (1H, m), 5.33-5.24 (2H, m), 4.70 (2H, s), 4.59-4.57 (2H, m), 3.77 (6H, s), 1.74-1.71 (2H, m), 1.49-1.45 (2H, m).

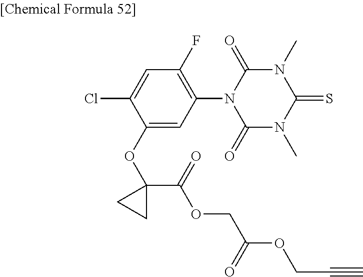 [SMILES: C#CCOC(=O)COC(=O)C1(Oc2cc(-n3c(=O)n(C)c(=S)n(C)c3=O)c(F)cc2Cl)CC1]

Compound of the Present Invention (I-19): ¹H-NMR (CDCl₃) δ:7.29 (1H, d), 7.20 (1H, d), 4.71 (2H, s), 4.69 (2H, d), 3.78 (6H, s), 2.50 (1H, m), 1.75-1.71 (2H, m), 1.50-1.46 (2H, m).

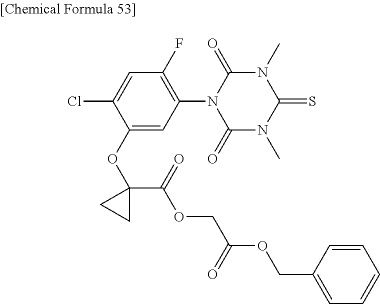 [SMILES: Cn1c(=S)n(C)c(=O)n(-c2cc(OC3(C(=O)OCC(=O)OCc4ccccc4)CC3)c(Cl)cc2F)c1=O]

Compound of the Present Invention (I-20): ¹H-NMR (CDCl₃) δ:7.37-7.36 (3H, m), 7.30-7.27 (3H, m), 7.23 (1H, d), 5.12 (2H, s), 4.72 (2H, s), 3.74 (6H, s), 1.74-1.71 (2H, m), 1.49-1.46 (2H, m).

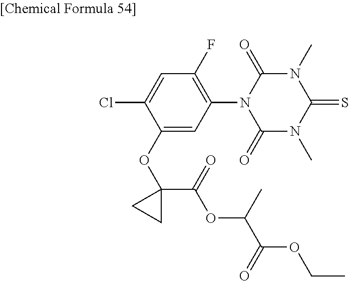 [SMILES: CCOC(=O)C(C)OC(=O)C1(Oc2cc(-n3c(=O)n(C)c(=S)n(C)c3=O)c(F)cc2Cl)CC1]

Compound of the Present Invention (I-21): ¹H-NMR (CDCl₃) δ:7.29-7.26 (2H, m), 5.07 (1H, q), 4.16-4.10 (2H, m), 3.77 (3H, s), 3.75 (3H, s), 1.73 (1H, m), 1.64 (1H, m), 1.50-1.38 (5H, m), 1.25 (3H, t).

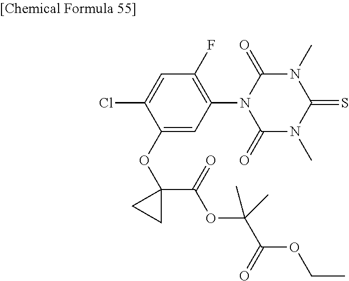 [SMILES: CCOC(=O)C(C)(C)OC(=O)C1(Oc2cc(-n3c(=O)n(C)c(=S)n(C)c3=O)c(F)cc2Cl)CC1]

Compound of the Present Invention (I-22): ¹H-NMR (CDCl₃) δ:7.28 (1H, d), 7.19 (1H, d), 4.13 (2H, q), 3.76 (6H, s), 1.65-1.61 (2H, m), 1.50 (6H, s), 1.42-1.38 (2H, m), 1.23 (3H, t).

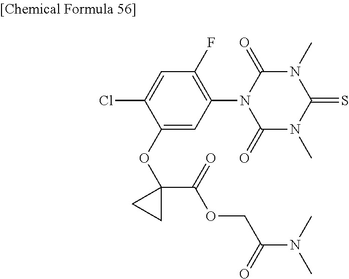 [SMILES: CN(C)C(=O)COC(=O)C1(Oc2cc(-n3c(=O)n(C)c(=S)n(C)c3=O)c(F)cc2Cl)CC1]

Compound of the Present Invention (I-24): ¹H-NMR (CDCl₃) δ:7.66 (1H, d), 7.25 (1H, d), 4.78 (2H, s), 3.75 (6H, s), 2.91 (3H, s), 2.86 (3H, s), 1.75-1.71 (2H, m), 1.46-1.42 (2H, m).

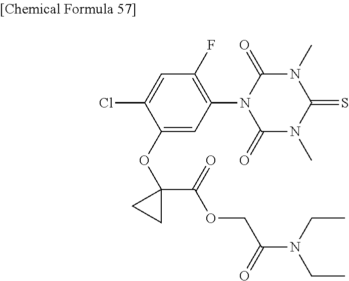 [SMILES: CCN(CC)C(=O)COC(=O)C1(Oc2cc(-n3c(=O)n(C)c(=S)n(C)c3=O)c(F)cc2Cl)CC1]

Compound of the Present Invention (I-25): ¹H-NMR (CDCl₃) δ:7.66 (1H, d), 7.25 (1H, d), 4.78 (2H, s), 3.75 (6H, s), 3.28 (2H, q), 3.18 (2H, q), 1.75-1.72 (2H, m), 1.45-1.42 (2H, m), 1.19 (3H, t), 1.07 (3H, t).

Examples of the compound of the present invention produced according to the above production method and production example are shown below.

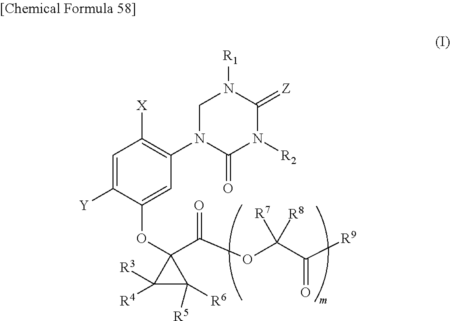

In the compounds represented by the formula (I), a compound wherein R¹ and R² are each a methyl group, R³, R⁴, R⁵, R⁶, R⁷, and R⁸ are each a hydrogen atom, X is a fluorine atom, Y is a chlorine atom, Z is a sulfur atom, m is 1, and R⁹ is any one described in [Table L1] (hereinafter, referred to as a compound group SX1).

In the compounds represented by the formula (I), a compound wherein R¹ and R² are each a methyl group, R³, R⁴, R⁵, and R⁶ are each a hydrogen atom, R⁷ is a hydrogen atom, R⁸ is a methyl group, X is a fluorine atom, Y is a chlorine atom, Z is a sulfur atom, m is 1, and R⁹ is any one described in [Table L1] (hereinafter, referred to as a compound group SX2).

In the compounds represented by the formula (I), a compound wherein R¹ and R² are each a methyl group, R³, R⁴, R⁵, and R⁶ are each a hydrogen atom, R⁷ and R⁸ are each a methyl group, X is a fluorine atom, Y is a chlorine atom, Z is a sulfur atom, m is 1, and R⁹ is any one described in [Table L1] (hereinafter, referred to as a compound group SX3).

In the compounds represented by the formula (I), a compound wherein R¹ and R² are each a methyl group, R³, R⁴, R⁵, and R⁶ are each a hydrogen atom, R⁷ is a hydrogen atom, R⁸ is an ethyl group, X is a fluorine atom, Y is a chlorine atom, Z is a sulfur atom, m is 1, and R⁹ is any one described in [Table L1] (hereinafter, referred to as a compound group SX4).

In the compounds represented by the formula (I), a compound wherein R¹ and R² are each a methyl group, R³, R⁴, R⁵, and R⁶ are each a hydrogen atom, R⁷ and R⁸ are each an ethyl group, X is a fluorine atom, Y is a chlorine atom, Z is a sulfur atom, m is 1, and R⁹ is any one described in [Table L1] (hereinafter, referred to as a compound group SX5).

In the compounds represented by the formula (I), a compound wherein R¹ and R² are each a methyl group, R³, R⁴, R⁵, and R⁶ are each a hydrogen atom, R⁷ and R⁸ are taken together with the carbon atom to which they are attached to form a cyclopropyl group, X is a fluorine atom, Y is a chlorine atom, Z is a sulfur atom, m is 1, and R⁹ is any one described in [Table L1] (hereinafter, referred to as a compound group SX6).

In the compounds represented by formula (I), a compound wherein R¹ is a methyl group, R² is an ethyl group, R³, R⁴, R⁵, R⁶, R⁷, and R⁸ are each a hydrogen atom, X is a fluorine atom, Y is a chlorine atom, Z is a sulfur atom, m is 1, and R⁹ is any one described in [Table L1] (hereinafter, referred to as a compound group SX7).

In the compounds represented by formula (I), a compound wherein R¹ is a methyl group, R² is an ethyl group, R³, R⁴, R⁵, and R⁶ are each a hydrogen atom, R⁷ is a hydrogen atom, R⁸ is a methyl group, X is a fluorine atom, Y is a chlorine atom, Z is a sulfur atom, m is 1, and R⁹ is any one described in [Table L1] (hereinafter, referred to as a compound group SX8).

In the compounds represented by formula (I), a compound wherein R¹ and R² are each a ethyl group, R³, R⁴, R⁵, R⁶, R⁷, and R⁸ are each a hydrogen atom, X is a fluorine atom, Y is a chlorine atom, Z is a sulfur atom, m is 1, and R⁹ is any one described in [Table L1] (hereinafter, referred to as a compound group SX9).

In the compounds represented by the formula (I), a compound wherein R¹ and R² are each an ethyl group, R³, R⁴, R⁵, and R⁶ are each a hydrogen atom, R⁷ is a hydrogen atom, R⁸ is a methyl group, X is a fluorine atom, Y is a chlorine atom, Z is a sulfur atom, m is 1, and R⁹ is any one described in [Table L1] (hereinafter, referred to as a compound group SX10).

In the compounds represented by the formula (I), a compound wherein R¹ and R² are each a methyl group, R³, R⁴, and R⁵ are each a hydrogen atom, R⁶ is a methyl group, R⁷ and R⁸ are each a hydrogen atom, X is a fluorine atom, Y is a chlorine atom, Z is a sulfur atom, m is 1, and R⁹ is any one described in [Table L1] (hereinafter, referred to as a compound group SX11).

In the compounds represented by the formula (I), a compound wherein R¹ and R² are each a methyl group, R³ is a hydrogen atom, R⁴ is a methyl group, R⁵ and R⁶ are each a hydrogen atom, R⁷ and R⁸ are each a hydrogen atom, X is a fluorine atom, Y is a chlorine atom, Z is a sulfur atom, m is 1, and R⁹ is any one described in [Table L1] (hereinafter, referred to as a compound group SX12).

In the compounds represented by the formula (I), a compound wherein R¹ and R² are each a methyl group, R³ and R⁴ are each a methyl group, R⁵ and R⁶ are each a hydrogen atom, R⁷ and R⁸ are each a hydrogen atom, X is a fluorine atom, Y is a chlorine atom, Z is a sulfur atom, m is 1, and R⁹ is any one described in [Table L1] (hereinafter, referred to as a compound group SX13).

In the compounds represented by the formula (I), a compound wherein R¹ and R² are each a methyl group, R³ and R⁴ are each a hydrogen atom, R⁵ and R⁶ are each a methyl group, R⁷ and R⁸ are each a hydrogen atom, X is a fluorine atom, Y is a chlorine atom, Z is a sulfur atom, m is 1, and R⁹ is any one described in [Table L1] (hereinafter, referred to as a compound group SX14).

In the compounds represented by the formula (I), a compound wherein R¹ and R² are each a methyl group, R³, R⁴, R⁵, and R⁶ are each a methyl group, R⁷ and R⁸ are each a hydrogen atom, X is a fluorine atom, Y is a chlorine atom, Z is a sulfur atom, m is 1, and R⁹ is any one described in [Table L1] (hereinafter, referred to as a compound group SX15).

In the compounds represented by formula (I), a compound wherein R¹ and R² are each a methyl group, R³, R⁴, R⁵, and R⁶ are each a hydrogen atom, X is a fluorine atom, Y is a chlorine atom, Z is a sulfur atom, m is 0, and R⁹ is any one described in [Table L1] (hereinafter, referred to as a compound group SX16).

In the compounds represented by the formula (I), a compound wherein R¹ and R² are each a methyl group, R³, R⁴, and R⁵ are each a hydrogen atom, R⁶ is a methyl group, X is a fluorine atom, Y is a chlorine atom, Z is a sulfur atom, m is 0, and R⁹ is any one described in [Table L1] (hereinafter, referred to as a compound group SX17).

In the compounds represented by the formula (I), a compound wherein R¹ and R² are each a methyl group, R³ and R⁴ are each a methyl group, R⁵ and R⁶ are each a hydrogen atom, X is a fluorine atom, Y is a chlorine atom, Z is a sulfur atom, m is 0, and R⁹ is any one described in [Table L1] (hereinafter, referred to as a compound group SX18).

In the compounds represented by the formula (I), a compound wherein R¹ and R² are each a methyl group, R³ and R⁴ are each a hydrogen atom, R⁵ and R⁶ are each a methyl group, X is a fluorine atom, Y is a chlorine atom, Z is a sulfur atom, m is 0, and R⁹ is any one described in [Table L1] (hereinafter, referred to as a compound group SX19).

In the compounds represented by formula (I), a compound wherein R¹ and R² are each a methyl group, R³, R⁴, R⁵, and R⁶ are each a methyl group, X is a fluorine atom, Y is a chlorine atom, Z is a sulfur atom, m is 0, and R⁹ is any one described in [Table L1] (hereinafter, referred to as a compound group SX20).

Next, formulation examples of the compound of the present invention will be described. Note that parts represent parts by weight. In addition, compounds of the present invention S represent compounds described in the compound groups SX1 to SX20.

### Formulation Example 1

35 parts of a mixture of polyoxyethylene alkyl ether sulfate ammonium salt and silica (weight ratio 1:1), 10 parts of any one of the compounds of the present invention S and 55 parts of water are mixed with each other, and the mixture is finely pulverized by wet grinding method, thereby obtaining a formulation.

### Formulation Example 2

50 parts of any one of the compounds of the present invention S, 3 parts of calcium lignosulfonate, 2 parts of sodium lauryl sulfate, and 45 parts of silica are pulverized and mixed, thereby obtaining a formulation.

### Formulation Example 3

5 parts of any one of the compounds of the present invention S, 9 parts of polyoxyethylene styrylphenyl ether, 5 parts of polyoxyethylene decyl ether (addition number of ethylene oxide: 5), 6 parts of calcium dodecylbenzenesulfonate, and 75 parts of xylene are mixed with each other, thereby obtaining a formulation.

### Formulation Example 4

2 parts of any one of the compounds of the present invention S, 1 part of silica, 2 parts of calcium lignosulfonate, 30 parts of bentonite, and 65 parts of kaolin clay are pulverized and mixed, and an appropriate amount of water is added to the obtained mixture. Thereafter, the mixture is kneaded, is granulated by a granulator, and is dried, thereby obtaining a formulation.

### Formulation Example 5

10 parts of any one of the compounds of the present invention S is mixed with a mixture of 18 parts of benzyl alcohol and 9 parts of DMSO, and 6.3 parts of GERONOL (registered trademark) TE250, 2.7 parts of Ethylan (registered trademark) NS-500LQ, and 54 parts of solvent naphtha are added to and mixed with the above-mentioned mixture, thereby obtaining a formulation.

### Formulation Example 6

0.1 parts of any one of the compounds of the present invention S and 39.9 parts of kerosene are mixed and dissolved, and the mixture is placed in an aerosol container. Thereafter, the container is filled with 60 parts of liquefied petroleum gas (mixture of propane, butane, and isobutane; and saturated vapor pressure: 0.47 MPa (25° C.)), thereby obtaining a formulation.

### Formulation Example 7

0.2 parts of any one of the compounds of the present invention S, 50 parts of pyrethrum extract cake powder, 30 parts of tab powder, and 19.8 parts of wood powder are mixed with each other, an appropriate amount of water is added to the obtained mixture, and the mixture is kneaded. Thereafter, the mixture is subjected to an extruder to form a plate-like sheet, and is formed into a spiral shape using a punching machine, thereby obtaining a formulation.

### Formulation Example 8

50 parts of any one of the compounds of the present invention S, 5 parts of sodium lignin sulfonate, 5 parts of polyoxyethylene alkyl ether, 5 parts of wet silica, and 35 parts of clay are sufficiently mixed with each other, thereby obtaining a formulation.

### Formulation Example 9

2 parts of sodium lignin sulfonate, 40 parts of talc, and 56.5 parts of bentonite are added to 1.5 parts of any one of the compounds of the present invention S, and are mixed with each other. Subsequently, an appropriate amount of water is added to the obtained mixture. Then, the mixture is further stirred, is granulated by a granulator, and is dried under forced air, thereby obtaining a formulation.

### Formulation Example 10

35 parts of a mixture of polyoxyethylene alkyl ether sulfate ammonium salt and wet silica (weight ratio 1:1), 10 parts of any one of the compounds of the present invention S, and 55 parts of water are sufficiently mixed with each other, thereby obtaining a formulation.

Next, the herbicidal efficacy of the compound of the present invention is shown by test examples. In the following test examples, the evaluation of the herbicidal efficacy is classified into 0 to 10, where if there is no or almost no difference in the state of emergence or growth of test weeds at the time of investigation as compared with that of test weeds in an untreated control, the evaluation of the herbicidal efficacy is classified as “0”, and if the test weeds are completely withered or emergence or growth of the test weeds is suppressed, the evaluation of the herbicidal efficacy is classified as “10”.

The evaluation of phytotoxicity is classified into 0 to 10, where if there is no or almost no difference in the state of emergence or growth of test crops at the time of investigation as compared with that of the untreated control, the evaluation of phytotoxicity is classified as “0”, and if the test crops are completely withered or emergence or growth of the test crops is completely suppressed, the evaluation of phytotoxicity is classified as “10”.

The untreated control means a section in which the same operation as that of the treated section is performed except that the test compound is not used.

### Test Method 1: Upland Field, Post-Emergence Foliar Application Test

A pot is filled with a commercially available culture soil, and seeds of velvetleaf (Abutilon theophrasti) are sown in the pot. Thereafter, the seeds are covered with approximately 0.5 cm of soil, and each of the seeds is cultivated in a greenhouse for 13 days. Next, a predetermined amount of the test compound is dissolved in 1 mL of a DMF solution containing 2% of an adjuvant, and 18 mL of water is added thereto, thereby preparing a diluent. The diluent is uniformly sprayed over plants so as to give the plants a predetermined treatment amount. Thereafter, the plants are cultivated in the greenhouse for 20 days, and herbicidal efficacy is evaluated.

### Test Example 1-1

As a result of performing, according to the test method 1, a test using the following compounds of the present invention as test compounds so that the treatment amount was 2 g/10,000 m², the following compounds of the present invention showed herbicidal efficacy of 9 or more.

Compounds of the present invention: I-1-1, I-2-1, I-1-2, I-5, I-6, I-7, I-12, I-13, I-14, I-15, I-16, I-17, I-18, I-20, I-21, I-22, I-24, and I-25

### Test Example 1-2

As a result of performing, according to the test method 1, a test using waterhemp (Amaranthus tuberculatus=A. rudis) instead of velvetleaf and using the following compounds of the present invention as test compounds so that the treated amount was 2 g/10,000 m², the compounds of the present invention described below showed herbicidal efficacy of 9 or more.

Compounds of the present invention: I-1-1, I-12, I-13, I-14, I-18, I-20, I-21, I-24, and I-25

### Test Method 2: Herbicidal Efficacy Test on Paddy Field Weeds

In a pot that has a diameter of 9 cm and a depth of 10 cm and is filled with steam-sterilized soil, Lindernia procumbens is seeded and cultivated up to one-leaf stage in a greenhouse. Next, a predetermined amount of the test compound is dissolved in 1 mL of a DMF solution containing 2% of an adjuvant, and 18 mL of water is added thereto, thereby preparing a diluent. The diluent is uniformly sprayed over the pot so as to give the pot a predetermined treatment amount. The next day, the plants are flooded with water to a depth of 3 cm and then are cultivated in a greenhouse for 20 days to evaluate herbicidal efficacy.

### Test Example 2-1

As a result of performing, according to the test method 2, a test using the following compounds of the present invention as test compounds so that the treatment amount was 16 g/10,000 m², the following compounds of the present invention showed herbicidal efficacy of 9 or more.

Compounds of the present invention: I-1-1, I-2-1, I-1-2, I-5, I-6, I-7, I-12, I-13, I-14, I-15, I-16, I-17, I-19, I-20, I-24, and I-25

Test Method 3: Upland Field, Post-Emergence Foliar Application Test A pot is filled with commercially available culture soil, and corn (Zea mays) seeds are sown in the pot. Thereafter, the seeds are covered with approximately 2 cm of soil, and each of the seeds is cultivated in a greenhouse for 13 days. Further, velvetleaf or waterhemp (Amaranthus tuberculatus=A. rudis) seeds are sown in the pot, and the seeds are covered with approximately 0.5 cm of soil. Thereafter, each of the seeds is cultivated in a greenhouse for 27 days or 17 days. Next, a predetermined amount of the test compound is dissolved in 0.95 mL of a DMF solution containing 2% of an adjuvant, and 8.55 mL of water is added thereto, thereby preparing a diluent. The diluent is uniformly sprayed over plants so as to give the plants a predetermined treatment amount. Thereafter, the plants are cultivated in the greenhouse for seven days, and phytotoxicity and herbicidal efficacy are evaluated.

### Test Example 3-1

A test was performed according to the test method 3 using, as a test compound, the following compounds of the present invention, a compound B-1, or a compound B-2 so that the treatment amount was 32 g/10,000 m² and 16 g/10,000 m². Table L4 shows phytotoxicity against corn and herbicidal efficacy against velvetleaf or waterhemp. As shown in Table L4, although the compounds of the present invention described showed the equivalent herbicidal efficacy to that of the compound B-1 or the compound B-2, phytotoxicity against corn was mild.

TABLE L4

| | | Phytotoxicity | Herbicidal efficacy | |
|---|---|---|---|---|
| | Dosage | Corn | Waterhemp | Velvetleaf |
| Compound | gAI/ha | 1 WAT | 1 WAT | 1 WAT |
| I-5 | 32 | 1 | 9 | 10 |
| | 16 | 1 | 9 | 10 |
| B-1 | 32 | 4 | 9 | 10 |
| | 16 | 3 | 9 | 10 |
| I-14 | 16 | 1 | 9 | 10 |
| | 8 | 1 | 9 | 10 |
| B-2 | 16 | 5 | 9 | 10 |
| | 8 | 3 | 9 | 10 |

In Table L4, a compound I-5 represents the compound of the present invention (I-5), and a compound I-14 represents the compound of the present invention (I-14).

In Table L4, the compound B-1 is a compound represented by the following formula B-1, and the compound B-2 is a compound represented by the following formula B-2.

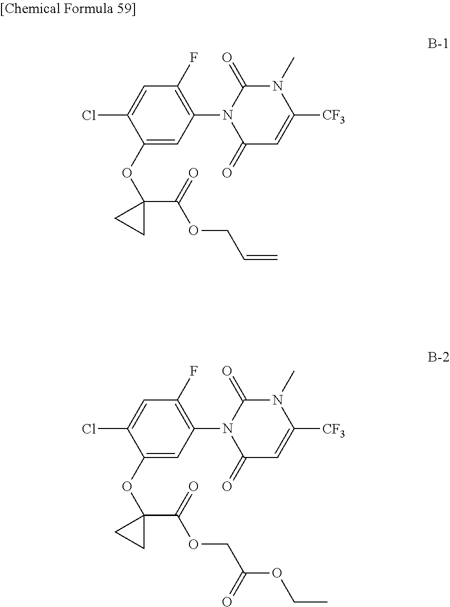 [SMILES: C=CCOC(=O)C1(Oc2cc(-n3c(=O)cc(C(F)(F)F)n(C)c3=O)c(F)cc2Cl)CC1 ; CCOC(=O)COC(=O)C1(Oc2cc(-n3c(=O)cc(C(F)(F)F)n(C)c3=O)c(F)cc2Cl)CC1]

In Table L4, gAI/ha represents the treatment dosage (g) of the compound of the present invention, the compound B-1 or the compound B-2 per hectare (ha) (=10,000 m²), and 1 WAT represents one week after treatment.

### Test Example 4-1

A test was performed according to the test method 1 using, as a test compound, the following compounds of the present invention, a compound C-1, or a compound C-2, or a compound C-3 so that the treatment amount was 2 g/10,000 m² and 1 g/10,000 m². Table L5 shows herbicidal efficacy against velvetleaf. As shown in Table L5, the compound of the present invention (I-5) and the compound of the present invention (I-14) shown in Table L5 exhibited more excellent herbicidal efficacy than that of the compounds C-1, C-2, and C-3.

TABLE L5

| | | Herbicidal |
|---|---|---|
| | | efficacy |
| | Dosage | Velvetleaf |
| Compound | gAI/ha | 3 WAT |
| I-5 | 2 | 10 |
| | 1 | 10 |
| C-1 | 2 | 1 |
| | 1 | 1 |
| C-2 | 2 | 1 |
| | 1 | 1 |
| I-14 | 2 | 10 |
| | 1 | 10 |
| C-3 | 2 | 1 |
| | 1 | 1 |

In Table L5, a compound I-5 represents the compound of the present invention (I-5), and a compound I-14 represents the compound of the present invention (I-14).

In Table L5, the compound C-1 is a compound represented by the following formula C-1, the compound C-2 is a compound represented by the following formula C-2, and the compound C-3 is a compound represented by the following formula C-3.

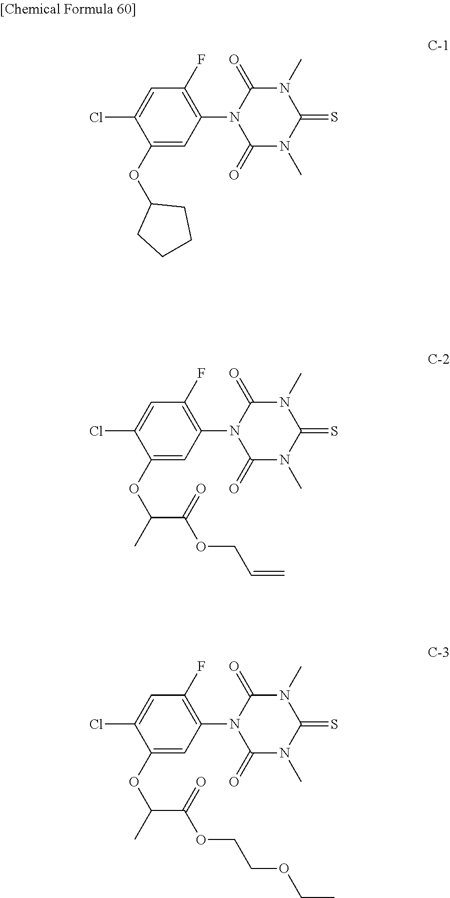 [SMILES: Cn1c(=S)n(C)c(=O)n(-c2cc(OC3CCCC3)c(Cl)cc2F)c1=O ; C=CCOC(=O)C(C)Oc1cc(-n2c(=O)n(C)c(=S)n(C)c2=O)c(F)cc1Cl ; CCOCCOC(=O)C(C)Oc1cc(-n2c(=O)n(C)c(=S)n(C)c2=O)c(F)cc1Cl]

In Table L5, gAI/ha represents the treatment dosage (g) of the compound of the present invention, the compound C-1, the compound C-2, or the compound C-3 per hectare (ha) (=10,000 m²), and 3 WAT represents three weeks after treatment.

### INDUSTRIAL APPLICABILITY

The compound of the present invention has a weed control effect.

1. A compound represented by a formula (I), or an N-oxide of the compound, or a salt of the compound or the N-oxide, 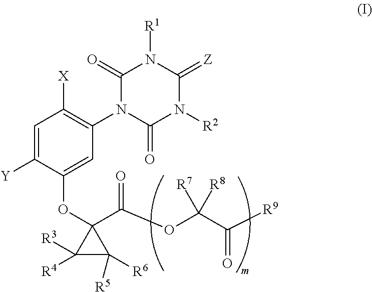
wherein
X represents a hydrogen atom or a halogen atom,
Y represents a halogen atom,
Z represents an oxygen atom or a sulfur atom,
R¹ and R² are the same as or different from each other and represent a hydrogen atom or a C1-C3 chain hydrocarbon group,
R³, R⁴, R⁵, and R⁶ are the same as or different from each other and represent a hydrogen atom or a C1-C3 alkyl group,
R⁷ and R⁸ are the same as or different from each other and represent a hydrogen atom or a C1-C3 alkyl group, or
R⁷ and R⁸ are taken together with a carbon atom to which they are attached to form a C3-C6 cycloalkyl group,
R⁹ represents —OR¹⁰, —SR¹⁰, or —NR¹¹R¹²,
R¹⁰ represents a C1-C8 chain hydrocarbon group optionally substituted with one or more substituents selected from a group A, a C3-C8 alicyclic hydrocarbon group optionally substituted with one or more substituents selected from a group B, or a hydrogen atom,
R¹¹ represents a hydrogen atom, a C1-C8 chain hydrocarbon group, or a C3-C8 alicyclic hydrocarbon group,
R¹² represents a C1-C8 chain hydrocarbon group optionally substituted with one or more substituents selected from the group A, a C3-C8 alicyclic hydrocarbon group optionally substituted with one or more substituents selected from the group B, a phenyl group optionally substituted with one or more substituents selected from a group C, a 5-6 membered aromatic heterocyclic group optionally substituted with one or more substituents selected from the group C, or a hydrogen atom, and
m represents 0 or 1;
The group A: a group consisting of a phenyl group optionally substituted with one or more substituents selected from the group C, a 5-6 membered aromatic heterocyclic group optionally substituted with one or more substituents selected from the group C, a halogen atom, a cyano group, a C1-C3 alkoxy group, and a C1-C3 alkylthio group;
The group B: a group consisting of an oxo group, a thioxo group, a halogen atom, a cyano group, a C1-C3 alkoxy group, and a C1-C3 alkylthio group;
The group C: a group consisting of a C1-C3 chain hydrocarbon group optionally substituted with one or more halogen atoms, a C1-C3 alkoxy group optionally substituted with one or more halogen atoms, a C1-C3 alkylthio group optionally substituted with one or more halogen atoms, a halogen atom, a cyano group, and a nitro group.

2. The compound, or the N-oxide of the compound, or the salt of the compound or the N-oxide according to claim 1, wherein Z is a sulfur atom.

3. The compound, or the N-oxide of the compound, or the salt of the compound or the N-oxide according to claim 1, wherein R³, R⁴, R⁵, and R⁶ are each a hydrogen atom.

4. The compound, or the N-oxide of the compound, or the salt of the compound or the N-oxide according to claim 1, wherein X is a fluorine atom, and Y is a chlorine atom.

5. The compound, or the N-oxide of the compound, or the salt of the compound or the N-oxide according to claim 1, wherein R¹ and R² are each a methyl group.

6. The compound, or the N-oxide of the compound, or the salt of the compound or the N-oxide according to claim 1, wherein
R⁷ and R⁸ are the same as or different from each other and represent a hydrogen atom, a methyl group or an ethyl group, or
R⁷ and R⁸ are taken together with a carbon atom to which they are attached to form a C3-C4 cycloalkyl group,
R¹⁰ is a C1-C3 chain hydrocarbon group optionally substituted with one or more substituents selected from the group A or a hydrogen atom,
R¹¹ is a hydrogen atom or a C1-C3 chain hydrocarbon group, and
R¹² is a C1-C3 chain hydrocarbon group optionally substituted with one or more substituents selected from the group A or a hydrogen atom.

7. The compound, or the N-oxide of the compound, or the salt of the compound or the N-oxide according to claim 1, wherein R⁹ is —OR¹⁰ or —NR¹¹R¹².

8. The compound, or the N-oxide of the compound, or the salt of the compound or the N-oxide according to claim 1, wherein R⁹ is —OR¹⁰.

9. The compound, or the N-oxide of the compound, or the salt of the compound or the N-oxide according to claim 1, wherein m is 0.

10. The compound, or the N-oxide of the compound, or the salt of the compound or the N-oxide according to claim 1, wherein m is 1.

11. An herbicidal composition comprising: the compound, or the N-oxide of the compound, or the salt of the compound or the N-oxide according to claim 1; and an inert carrier.

12. A method of controlling a weed, comprising a step of applying, to the weed or soil in which the weed grows, the compound, or the N-oxide of the compound, or the salt of the compound or the N-oxide according to claim 1.

13. Use of the compound, or the N-oxide of the compound, or the salt of the compound or the N-oxide according to claim 1, for controlling a weed.

14. A composition comprising: one or more components selected from a group consisting of a group (d) and a group (f); and the compound, or the N-oxide of the compound, or the salt of the compound or the N-oxide according to claim 1,
the group (d): phytotoxicity alleviating ingredients,
the group (f): herbicidally active ingredients.
